(19) United States
(12) Patent Application Publication
(10) Pub. No.: US 2024/0327429 A1
(43) Pub. Date: Oct. 3, 2024

(54) [1,3]Thiazolo[4,5-d]-pyrimidon-7-ones as inhibitors of NOX4

(71) Applicant: Boehringer Ingelheim International GmbH, Ingelheim am Rheim (DE)

(72) Inventors: Christian GNAMM, Bieberach an der Riss (DE); Elmar BAUSCHATZ, Schemmerhofen (DE); Frank BUETTNER, Attenweiler (DE); Cédrickx GODBOUT, Schemmerhofen (DE); Annekatrin Charlotte HEIMANN, Biberach an der Riss (DE); Christoph HOENKE, Biberach an der Riss (DE); Ferenc KONTES, Biberach (DE); Christian Andreas KUTTRUFF, Donaueschingen (DE); Dieter WIEDENMAYER, Biberach an der Riss (DE); Raphael WILDERMUTH, Irsee (DE)

(21) Appl. No.: 18/588,515

(22) Filed: Feb. 27, 2024

(30) Foreign Application Priority Data
Feb. 28, 2023 (EP) 23159 067.0

(51) Int. Cl. C07D 513/04; A61K 31/519; A61K 45/06

(52) U.S. Cl. CPC C07D 513/04; A61K 31/519; A61K 45/06

(57) ABSTRACT

The present disclosure provides [1,3]thiazolo[4,5-d]-pyrimidon-7-ones that are inhibitors of NOX4, and are therefore useful for the treatment of diseases treatable by inhibition of NOX4. Also provided are pharmaceutical compositions containing the same, and processes for preparing said compounds.

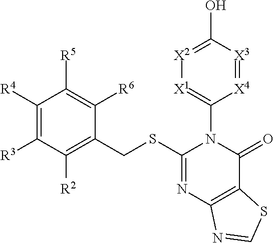

### TECHNICAL FIELD

The present disclosure provides certain thiazolo-pyrimidinone derivatives that are inhibitors of NOX4, and are therefore useful for the treatment of diseases treatable by inhibition of NOX4. Furthermore, the invention relates to pharmaceutical compositions and combinations comprising these compounds, as well as their use in methods for the treatment of diseases associated with or modulated by NOX4. Particularly, the pharmaceutical compositions of the invention are suitable for the therapy of interstitial lung diseases, e.g. idiopathic pulmonary disease, and may also be suitable for the therapy of fibrotic diseases, allergic and inflammatory diseases.

### BACKGROUND INFORMATION

Redox signaling is a critical part of a variety of cell signaling pathways involved in the regulation of cell growth, differentiation, metabolism, immune regulation, and other physiological functions. It is characterized by an oxidation-reduction reaction or covalent adduct formation between the sensor and the second messenger. Thereby oxidation of the sensor protein can modulate its activity, conformation, or susceptibility towards degradation.

In case of redox signaling the second messengers consist of a group of reactive oxygen and nitrogen species (ROS, RNS). These are generated as side products during metabolic reactions in the mitochondria but also by specialized enzymes like NADPH-oxidases (NOXes). In parallel, a complex antioxidant system evolved to protect the cells from injury by ROS and RNS, ensuring a tight balance between ROS/RNS formation and degradation.

In case this balance is disturbed, oxidative stress can occur leading to increased protein oxidation and aberrant redox signaling. Therefore, oxidative stress is associated with various pathophysiological conditions like interstitial lung diseases, cancer and inflammatory diseases.

Several studies showed over the last decade that in interstitial lung diseases, like idiopathic pulmonary fibrosis (IPF), the redox balance is disturbed¹ and that NOX4 is specifically upregulated in the lungs of IPF patients¹. IPF patients experience a decline in their lung function and blood oxygenation, which is caused by the remodeling of the lung parenchyma in combination with the stiffening of the lung tissue due to increased matrix deposition. Upon epithelial injury due to e.g. infection, air pollutants etc., fibroblasts are activated to enable migration of immune cells as well as epithelial precursor cells to the site of injury. Once the injury is resolved fibroblasts as well as immune cells undergo apoptosis to allow complete restoration of the tissue. In IPF an aberrant repair is occurring leading to a progressive remodeling of the lung parenchyma². Recent studies using single cell sequencing allowed the identification and comparison of different cell types of lung tissue derived from IPF patients or healthy controls. These studies showed increased numbers of fibroblasts as well as a strong dedifferentiation of the epithelial cell population³.

NOX4 is part of the NADPH oxidase enzyme family. NOXes are membrane bound multi-subunit protein complexes, which transfer electrons across the plasma membrane to generate ROS. While the activity of all other family members (NOX1, NOX2, NOX3 and NOX5) is tightly controlled and inducible, NOX4 is the only family member that is constitutively active¹. It is localized in the plasma membrane, perinuclear vesicles, the endoplasmatic reticulum (ER), the mitochondria and the nuclear membrane. The generation of hydrogen peroxide (H₂O₂) by NOX4 leads to reversible oxidation of cysteine thiol groups and/or S-glutathionylation¹. Thereby, NOX4 is modulating TGFβ signaling and other key pathways known to be involved in disease progression¹.

IPF fibroblasts show higher expression of NOX4 in comparison to fibroblasts isolated from healthy tissue, as well as hyperplastic alveolar cells. The role of NOX4 in fibroblast activation as well as proliferation was investigated in lung fibroblasts¹. Fibroblast proliferation, as well as fibroblast activation are known processes driving disease progression. Immunohistochemistry on control and IPF lung samples confirmed increased NOX4 staining in fibroblasts and showed stronger staining in bronchial and alveolar epithelial cells¹.

Acute respiratory distress syndrom (ARDS) and acute lung injury (ALI) are characterized by pulmonary infiltration and edema. Literature shows that NOX4 is increased in patients and plays a crucial role in cell recruitment in animal models⁴.

Non-alcoholic steatohepatitis (NASH) is characterized by an increase in lipid content and inflammation in the liver. The chronic feedback loop of lipotoxicity, increased inflammation and cell death leads to liver fibrosis. Throughout that process liver cells, namely stellate cells, dedifferentiate to an activated phenotype, of which one hallmark is the induction of pro-fibrotic and pro-inflammatory expression pattern. As on main redox-mediating signalling pathway, the NOX-family—especially NOX4—plays a crucial role in that feedback loop. NOX4-dependent signalling directly leads to an induction of NFkB and MAPk transcription leading to induction of gene products, such as smooth muscle actin (SMA), collagen or tumor necrosis factor (TNF).

Published literature shows that NOX4 is expressed in stellate cells and is expressed through the process of stellate cell activation. Vice versa, the inhibition of NOX4 on the other hand leads to attenuated expression of stellate cell-activation markers.

In addition to in vitro analyses, studies in NOX4 knock-out mice show reduced fibrosis and inflammation in NASH-relevant models.

Aside from the prevention of fibrosis onset and progression, NOX4 plays a crucial role of endothelial integrity. Thereby, NOX4 activity fosters the nitric oxide (NO) production, leading to vascular relaxation and reduced endothelial inflammation. Following, NOX4 inhibition has a beneficial impact on portal hypertension.

In cancer, ROS deregulation contributes to tumor development, progression, and metastasis. In some instances, this enhanced ROS, driven by various oncogenic perturbations, is required for tumorigenicity leading to the acquisition of further DNA damage and genome instability in cancer cells⁵. Increased ROS occurs through a variety of mechanisms such as increased expression of the NOX proteins or NOX activators, or the downregulation of ROS-regulating systems. NOX4 itself has been reported to be increased in many types of tumors leading to increased proliferation, migration, and apoptosis⁵. Tumor associated macrophages are influenced by ROS and can adopt an immunosuppressive phenotype within tumors in response to ROS⁵. Furthermore, when fibroblasts are educated to become cancer associated fibroblasts (CAFs), NOX4 is upregulated and leads to CD8+ T cell exclusion and immune suppression in tumors. Deletion or inhibition of NOX4 in several mouse models of cancer, has reversed this immune suppression and restored immunotherapy response to anti-PD-1 therapy (PD1 being programmed cell death protein 1)⁵. Therefore, specific inhibition of NOX proteins in tumor cells themselves or stromal cells, is a tractable target for anti-tumor therapies.

The list of the references cited above can be found here:
1. Veith, C., Boots, A. W., Idris, M., Schooten, F. J. van & Vliet, A. van der. Redox Imbalance in Idiopathic Pulmonary Fibrosis: A Role for Oxidant Cross-Talk Between NADPH Oxidase Enzymes and Mitochondria. Antioxid Redox Sign 31, 1092-1115 (2019).
2. Hinz, B. & Lagares, D. Evasion of apoptosis by myofibroblasts: a hallmark of fibrotic diseases. Nat Rev Rheumatol 16, 11-31 (2020).
3. Habermann, A. C. et al. Single-cell RNA sequencing reveals profibrotic roles of distinct epithelial and mesenchymal lineages in pulmonary fibrosis. Sci Adv 6, eabal972 (2020).
4. Lee, S. H. et al. NADPH oxidase 4 signaling in a ventilator-induced lung injury mouse model. Respir Res 23, 73 (2022).
5. Cheung, E. C. & Vousden, K. H. The role of ROS in tumour development and progression. Nat Rev Cancer 22, 280-297 (2022).

### DETAILED DESCRIPTION OF THE INVENTION

The present invention discloses novel thiazolo-pyrimidinone derivatives that are inhibitors of NOX4, possessing appropriate pharmacological and pharmacokinetic properties enabling their use as medicaments for the treatment of conditions and/or diseases treatable by inhibition of NOX4.

In the prior art, WO2016207785 discloses benzoxazole and benzthiazole based NOX4 inhibitors with the following generalized structural formula

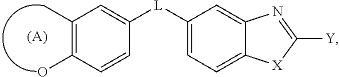

of which example 1, example 15 and example 80 are reported with the following IC₅₀ values in an assay on modified human embryonic kidney (HEK) cells using Amplex Red as detecting reagent:

| Example as | | NOX4 IC₅₀ [μM] as |
|---|---|---|
| disclosed | | disclosed in |
| in WO2016207785 | Structure | WO2016207785 |
| 1 | 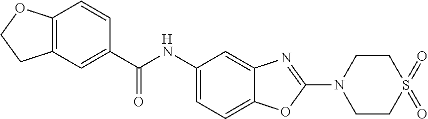 | 0.34 |
| 15 | 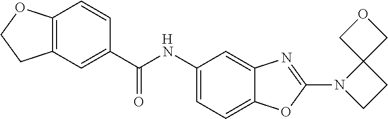 | 0.40 |
| 80 | 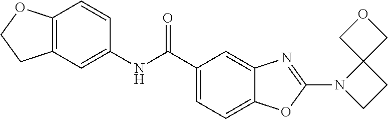 [SMILES: O=C(Nc1ccc2c(c1)CCO2)c1ccc2oc(N3CCC34COC4)nc2c1] | 0.31 |

When tested in an assay for metabolic stability as described herein below, examples 1, 15 and 80 show a clearance in human hepatocytes of 52% QH, 29% QH and 46% QH, respectively.

Another patent application, WO2005049613 discloses bicyclic pyrimidin-4-(3H)-one based modulators of the vanilloid-1 receptor (VR1) of the following generalized structural formula

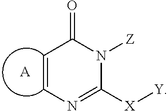

Examples 19, 20, 30, 59 and 63 from WO2005049613 have been tested for their activity on NOX4 inhibition with the assay described herein below, resulting in the following IC₅₀ values:

| Example as | | NOX4 |
|---|---|---|
| disclosed in | | IC₅₀ |
| WO2005049613 | Structure | [μM] |
| 19 | 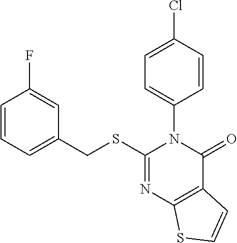 [SMILES: O=c1c2ccsc2nc(SCc2cccc(F)c2)n1-c1ccc(Cl)cc1] | >99 |
| 20 | 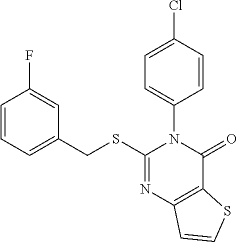 [SMILES: O=c1c2sccc2nc(SCc2cccc(F)c2)n1-c1ccc(Cl)cc1] | >99 |
| 30 | 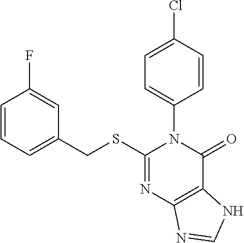 [SMILES: O=c1c2[nH]cnc2nc(SCc2cccc(F)c2)n1-c1ccc(Cl)cc1] | >99 |
| 59 | 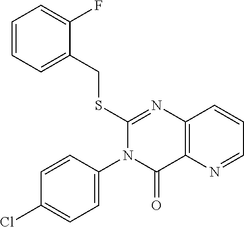 [SMILES: O=c1c2ncccc2nc(SCc2ccccc2F)n1-c1ccc(Cl)cc1] | 79 |
| 63 | 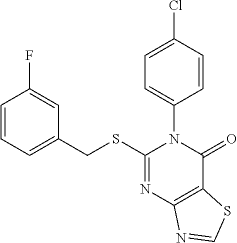 [SMILES: O=c1c2scnc2nc(SCc2cccc(F)c2)n1-c1ccc(Cl)cc1] | 55 |

As visible from the above data, the tested compounds of WO2005049613 show low activity as inhibitors of NOX4.

Surprisingly it was found that the exchange of the para-chloro substituent in example 63 from WO2005049613 with a para-hydroxy substituent increases the inhibition of NOX4 from 55 μM to 0.66 μM with respect to IC₅₀ as tested in the NOX4-inhibition assay described herein below, as shown by example 1 of the present invention in the following table:

| | | NOX4 |
|---|---|---|
| | | IC₅₀ |
| Example | Structure | [μM] |
| 1 | 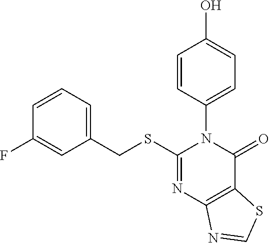 [SMILES: O=c1c2scnc2nc(SCc2cccc(F)c2)n1-c1ccc(O)cc1] | 0.56 |

The effect of the hydroxy group in this position on the NOX4 inhibitory activity of other compounds of the present invention is also shown in the following table:

| | | NOX4 |
|---|---|---|
| | | IC₅₀ |
| Example | Structure | [μM] |
| Reference | 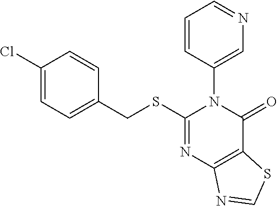 [SMILES: O=c1c2scnc2nc(SCc2ccc(Cl)cc2)n1-c1cccnc1] | 5.45 |
| 25 | 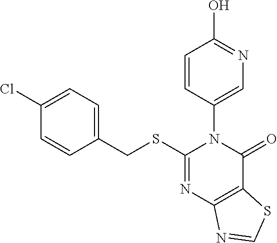 [SMILES: O=c1c2scnc2nc(SCc2ccc(Cl)cc2)n1-c1ccc(O)nc1] | 0.10 |
| Reference | 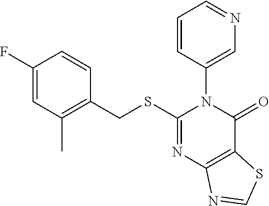 [SMILES: Cc1cc(F)ccc1CSc1nc2ncsc2c(=O)n1-c1cccnc1] | 8.12 |
| 29 | 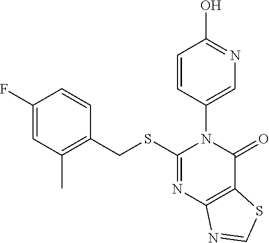 [SMILES: Cc1cc(F)ccc1CSc1nc2ncsc2c(=O)n1-c1ccc(O)nc1] | 0.15 |
| Reference | 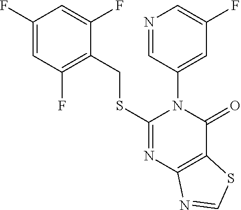 [SMILES: O=c1c2scnc2nc(SCc2c(F)cc(F)cc2F)n1-c1cncc(F)c1] | 1.65 |
| 31 | 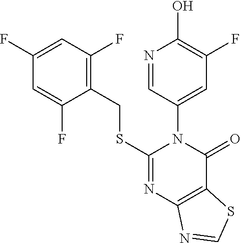 [SMILES: O=c1c2scnc2nc(SCc2c(F)cc(F)cc2F)n1-c1cnc(O)c(F)c1] | 0.06 |

It is therefore the aim of the present invention to provide thiazolo-pyrimidinones which are potent, metabolically stable and selective NOX4 inhibitors.

The compounds of the present invention according to general formula (I)

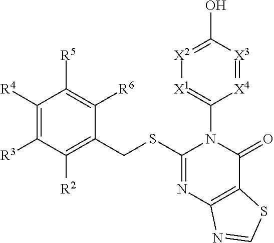

wherein
X¹, X², X⁴ are, independently of each other, N or C—R¹;
X³ is N or C—R^{1.1};
provided that not more than two of X¹, X², X³, X⁴ are N simultaneously;
R¹ is selected from among a group consisting of —H and -halogen;
R^{1.1} is selected from among a group consisting of —H, -halogen, —CN;
R², R³, R⁴, R⁵, R⁶ are, independently of each other, selected from among a group consisting of —H, -halogen, —CF₃ and -methyl;
or salts thereof, exhibit several advantageous properties such as high potency, high metabolic stability and high selectivity for NOX4 and against NOX1, NOX2, NOX3 and NOX5.

The compounds according to the invention typically show inhibition of NOX4 with IC₅₀-values below 600 nM, preferably below 400 nM, more preferably below 200 nM, most preferably below 100 nM (see assay description herein below and table 1). High potency can enable lower doses for pharmacological efficacy. Lower doses have the advantages of lower “drug load” or “drug burden” (parent drug and metabolites thereof) for the patient causing potentially less side effects, and lower production costs for the drug product.

In a further aspect of the invention, the compounds according to the invention are selective NOX4-inhibitors, and are selective against NOX1, NOX2, NOX3 and NOX5. Preferred are compounds in which the IC₅₀ on NOX4 is 10× lower than the IC₅₀ on either of NOX1, NOX2, NOX3 or NOX5. More preferred are compounds in which the IC₅₀ on NOX4 is 30× lower than the IC₅₀ on either of NOX1, NOX2, NOX3 or NOX5. Most preferred are compounds in which the IC₅₀ on NOX4 is 100× lower than the IC₅₀ on either of NOX1, NOX2, NOX3 or NOX5 (see assay description herein below and table 2).

Furthermore, the compounds according to the invention are metabolically stable as shown in human hepatocytes. Metabolic stability in human hepatocytes in this respect is defined as below or equal to 45% QH, preferably below or equal to 30% QH, more preferably below or equal to 20% QH (see assay description herein below and table 3 and the definition of how to calculate the % QH=hepatic blood flow herein below). Therefore, the compounds of the present invention are expected to have a favorable in vivo clearance and thus the favourably long duration of action in humans. Stability in human hepatocytes refers to the susceptibility of compounds to biotransformation in the context of selecting and/or designing drugs with favorable pharmacokinetic properties, as the primary site of metabolism for many drugs is the liver. Human hepatocytes contain the cytochrome P450 (CYPs) and additional enzymes for phase II metabolism (e.g. phosphatases and sulfatases), and thus represent a model system for studying in vitro how a drug is metabolised. Stability in hepatocytes is associated with several advantages, including improved bioavailability and half-life, which can allow lower and less frequent dosing in patients. Thus, stability in hepatocytes is a favorable characteristic for compounds that are to be used as drugs in the treatment of a disease.

### Used Terms and Definitions

### General Definitions

Terms not specifically defined herein should be given the meanings that would be given to them by one of skill in the art in light of the disclosure and the context. As used in the specification, however, unless specified to the contrary, the following terms have the meaning indicated and the following conventions are adhered to.

In the groups, radicals, or moieties defined below, the number of carbon atoms is often specified preceding the group, for example, C₁₋₆-alkyl means an alkyl group or radical having 1 to 6 carbon atoms. In general in groups like HO, H₂N, (O)S, (0)₂S, NC (cyano), HOOC, F₃C or the like, the skilled artisan can see the radical attachment point(s) to the molecule from the free valences of the group itself. For combined groups comprising two or more subgroups, the last named subgroup is the radical attachment point, for example, the substituent “aryl-C₁₋₃-alkylene” means an aryl group which is bound to a C₁₋₃-alkyl-group, the latter of which is bound to the core or to the group to which the substituent is attached. In case a compound of the present invention is depicted in the form of a chemical name and as a formula, in case of any discrepancy the formula shall prevail. A wavy line may be used in sub-formulas to indicate the bond which is connected to the core molecule as defined. For example, the term “3-carboxypropyl-group” represents the following substituent:

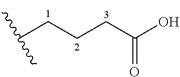 [SMILES: *CCCC(=O)O]

wherein the carboxy group is attached to the third carbon atom of the propyl group. The terms “1-methylpropyl-”, “2,2-dimethylpropyl-” or “cyclopropylmethyl-” group represent the following groups:

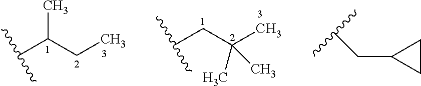

The wavy line may be used in sub-formulas to indicate the bond which is connected to the core molecule as defined.

### Term Substituted

The term “substituted” as used herein, means that one or more hydrogens on the designated atom are replaced by a group selected from a defined group of substituents, provided that the designated atom's normal valence is not exceeded, and that the substitution results in a stable compound. Likewise, the term “substituted” may be used in connection with a chemical moiety instead of a single atom, e.g. “substituted alkyl”, “substituted aryl” or the like.

Unless specifically indicated, throughout the specification and the appended claims, a given chemical formula or name shall encompass tautomers and all stereo, optical and geometrical isomers (e.g. enantiomers, diastereomers, E/Z isomers etc. . . . ) and racemates thereof as well as mixtures in different proportions of the separate enantiomers, mixtures of diastereomers, or mixtures of any of the foregoing forms where such isomers and enantiomers exist, as well as solvates thereof such as for instance hydrates.

Unless specifically indicated, also “pharmaceutically acceptable salts” as defined in more detail below shall encompass solvates thereof such as for instance hydrates.

### Salts

The phrase “pharmaceutically acceptable” is employed herein to refer to those compounds, materials, compositions, and/or dosage forms which are, within the scope of sound medical judgment, suitable for use in contact with the tissues of human beings without excessive toxicity, irritation, allergic response, or other problem or complication, and commensurate with a reasonable benefit/risk ratio.

As used herein, “pharmaceutically acceptable salt” refers to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like.

For example, such salts include salts from benzenesulfonic acid, benzoic acid, citric acid, ethanesulfonic acid, fumaric acid, gentisic acid, hydrobromic acid, hydrochloric acid, maleic acid, malic acid, malonic acid, mandelic acid, methanesulfonic acid, 4-methyl-benzenesulfonic acid, phosphoric acid, salicylic acid, succinic acid, sulfuric acid and tartaric acid. Further pharmaceutically acceptable salts can be formed with cations from ammonia, L-arginine, calcium, 2,2′-iminobisethanol, L-lysine, magnesium, N-methyl-D-glucamine, potassium, sodium and tris(hydroxymethyl)-aminomethane.

The pharmaceutically acceptable salts of the present invention can be synthesized from the parent compound which contains a basic or acidic moiety by conventional chemical methods. Generally, such salts can be prepared by reacting the free acid or base forms of these compounds with a sufficient amount of the appropriate base or acid in water or in an organic diluent such as ether, ethyl acetate, ethanol, isopropanol, or acetonitrile, or a mixture thereof.

Salts of other acids than those mentioned above which for example are useful for purifying or isolating the compounds of the present invention (e.g. trifluoro acetate salts,) also comprise a part of the invention.

### Halogen

The term halogen denotes fluorine, chlorine, bromine and iodine.

### Alkyl

The term “C₁₋ₙ-alkyl”, wherein n is an integer selected from 2, 3, 4, 5 or 6, preferably 4, 5, or 6, either alone or in combination with another radical, denotes an acyclic, saturated, branched or linear hydrocarbon radical with 1 to n C atoms. For example the term C₁₋₅-alkyl embraces the radicals H₃C—, H₃C—CH₂—, H₃C—CH₂—CH₂—, H₃C—CH(CH₃)—, H₃C—CH₂—CH₂—CH₂—, H₃C—CH₂—CH(CH₃)—, H₃C—CH(CH₃)—CH₂—, H₃C—C(CH₃)₂—, H₃C—CH₂—CH₂—CH₂—CH₂—, H₃C—CH₂—CH₂—CH(CH₃)—, H₃C—CH₂—CH(CH₃)—CH₂—, H₃C—CH(CH₃)—CH₂—CH₂—, H₃C—CH₂—C(CH₃)₂—, H₃C—C(CH₃)₂—CH₂—, H₃C—CH(CH₃)—CH(CH₃)— and H₃C—CH₂—CH(CH₂CH₃)—.

### Alkylene

The term “C₁₋ₙ-alkylene” wherein n is an integer selected from 2, 3, 4, 5 or 6, preferably 4, 5 or 6, either alone or in combination with another radical, denotes an acyclic, saturated, branched or linear chain divalent alkyl radical containing from 1 to n carbon atoms. For example the term C₁₋₄-alkylene includes —CH₂—, —CH₂—CH₂—, —CH(CH₃)—, —CH₂—CH₂—CH₂—, —C(CH₃)₂—, —CH(CH₂CH₃)—, —CH(CH₃)—CH₂—, —CH₂—CH(CH₃)—, —CH₂—CH₂—CH₂—CH₂—, —CH₂—CH₂—CH(CH₃)—, —CH(CH₃)—CH₂—CH₂—, —CH₂—CH(CH₃)—CH₂—, —CH₂—C(CH₃)₂—, —C(CH₃)₂—CH₂—, —CH(CH₃)—CH(CH₃)—, —CH₂—CH(CH₂CH₃)—, —CH(CH₂CH₃)—CH₂—, —CH(CH₂CH₂CH₃)—, —CH(CH(CH₃))₂— and —C(CH₃)(CH₂CH₃)—.

### Alkenyl

The term “C₂₋ₘ-alkenyl” is used for a group “C₂₋ₘ-alkyl” wherein m is an integer selected from 3, 4, 5 or 6, preferably 4, 5 or 6, if at least two carbon atoms of said group are bonded to each other by a double bond.

### Alkenylene

The term “C₂₋ₘ-alkenylene” is used for a group “C₂₋ₘ-alkylene”, wherein m is an integer selected from 3, 4, 5 or 6, preferably 4, 5 or 6, if at least two carbon atoms of said group are bonded to each other by a double bond.

### Alkynyl

The term “C₂₋ₘ-alkynyl” is used for a group “C₂₋ₘ-alkyl” wherein m is an integer selected from 3, 4, 5 or 6, preferably 4, 5 or 6, if at least two carbon atoms of said group are bonded to each other by a triple bond.

### Alynylene

The term “C₂₋ₘ-alkynylene” is used for a group “C₂₋ₘ-alkylene” wherein m is an integer selected from 3, 4, 5 or 6, preferably 4, 5 or 6, if at least two of those carbon atoms of said group are bonded to each other by a triple bond.

### Cycloalkyl

The term “C₃₋ₖ-cycloalkyl”, wherein k is an integer selected from 3, 4, 5, 7 or 8, preferably 4, 5 or 6, either alone or in combination with another radical, denotes a cyclic, saturated, unbranched hydrocarbon radical with 3 to k C atoms. For example the term C₃₋₇-cycloalkyl includes cyclopropyl, cyclobutyl, cyclopentyl, cyclohexyl and cycloheptyl.

### Cycloalkenyl

The term “C₃-k-cycloalkenyl”, wherein k is an integer selected from 3, 4, 5, 7 or 8, preferably 4, 5 or 6, either alone or in combination with another radical, denotes a cyclic, unsaturated, but non-aromatic, unbranched hydrocarbon radical with 3 to k C atoms, at least two of which are bonded to each other by a double bond. For example the term C₃₋₇-cycloalkenyl includes cyclopropenyl, cyclobutenyl, cyclopentenyl, cyclopentadienyl, cyclohexenyl, cyclohexadienyl, cycloheptenyl, cycloheptadienyl and cycloheptatrienyl.

### Halo-(Alkyl, Alkylene or Cycloalkyl)

The term “halo” added to an “alkyl”, “alkylene” or “cycloalkyl” group (saturated or unsaturated) defines an alkyl, alkylene or cycloalkyl group wherein one or more hydrogen atoms are replaced by a halogen atom selected from among fluorine, chlorine or bromine, preferably fluorine and chlorine, particularly preferred is fluorine. Examples include: F₃C—, H₂FC—, HF₂C.

### Carbocyclyl

The term “carbocyclyl”, either alone or in combination with another radical, means a mono-, bi- or tricyclic ring structure consisting of 3 to 14 carbon atoms. The term “carbocyclyl” refers to fully saturated, partially saturated and aromatic ring systems. The term “carbocyclyl” encompasses fused, bridged and spirocyclic systems.

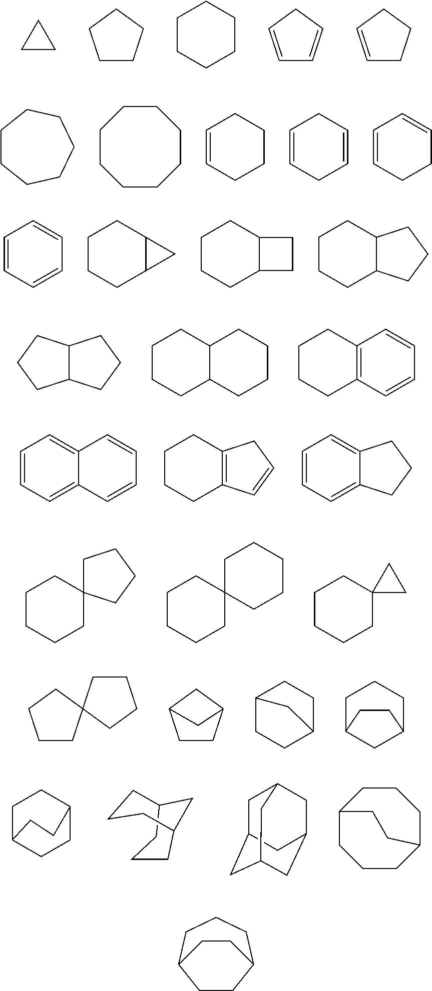

### Heterocyclyl

The term “heterocyclyl” means a saturated or unsaturated mono- or polycyclic ring system optionally comprising aromatic rings, containing one or more heteroatoms selected from N, O, S, SO or SO₂ consisting of 3 to 14 ring atoms wherein none of the heteroatoms is part of the aromatic ring. The term “heterocyclyl” is intended to include all the possible isomeric forms.

Thus, the term “heterocyclyl” includes the following exemplary structures (not depicted as radicals as each form is optionally attached through a covalent bond to any atom so long as appropriate valences are maintained):

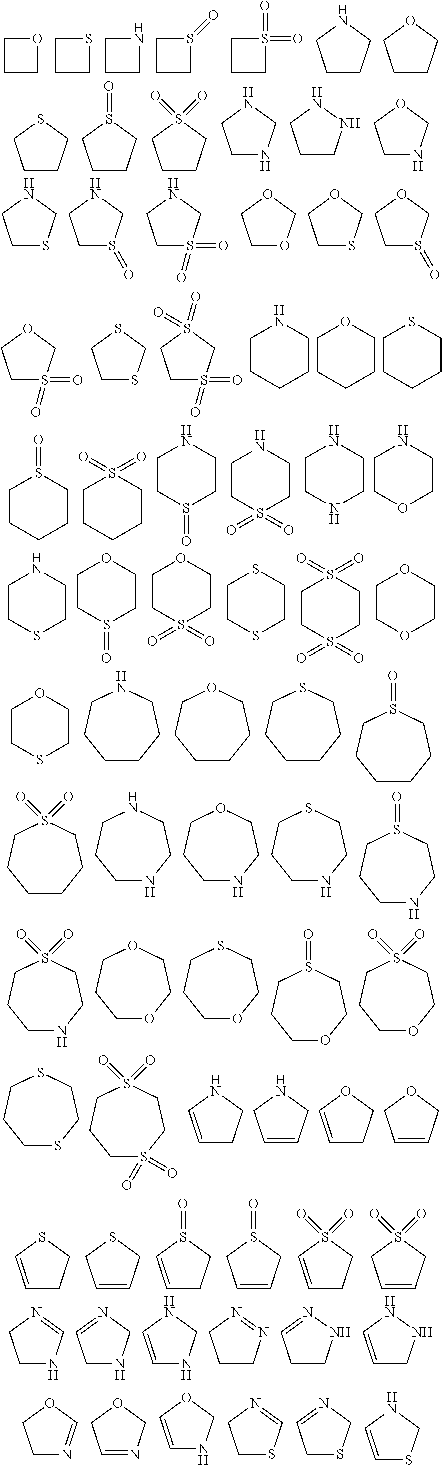

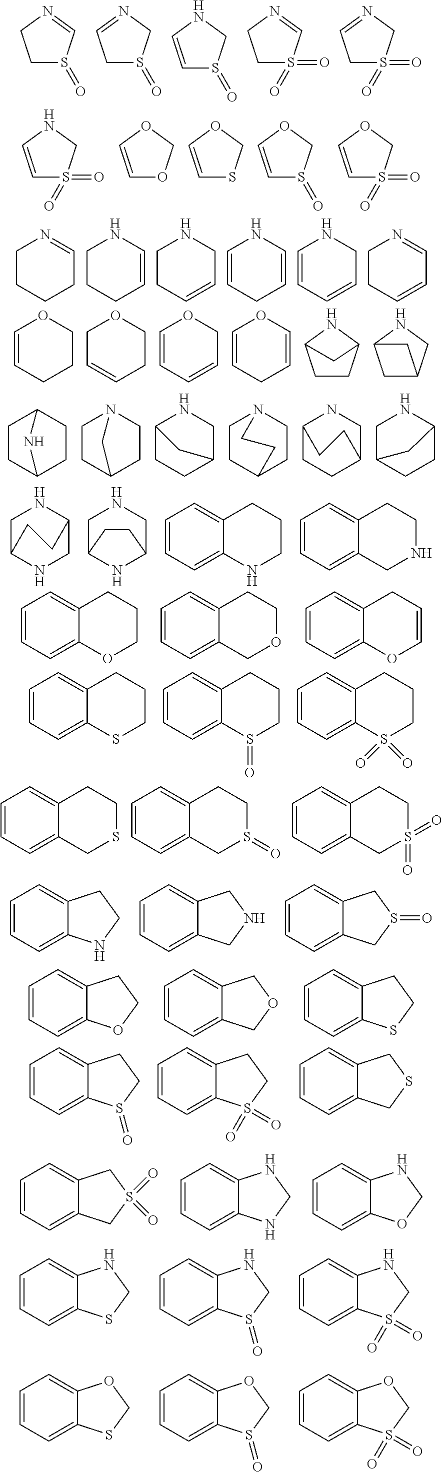

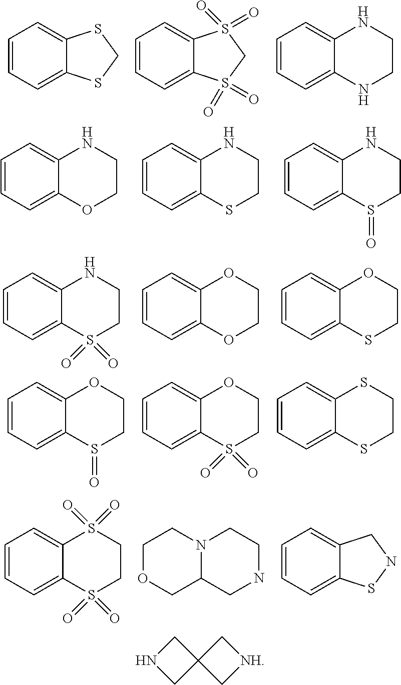

### Heteroaryl

The term “heteroaryl” means a mono- or polycyclic ring system, comprising at least one aromatic ring, containing one or more heteroatoms selected from N, O, S, SO or SO₂, consisting of 5 to 14 ring atoms wherein at least one of the heteroatoms is part of an aromatic ring. The term “heteroaryl” is intended to include all the possible isomeric forms.

Thus, the term “heteroaryl” includes the following exemplary structures (not depicted as radicals as each form is optionally attached through a covalent bond to any atom so long as appropriate valences are maintained):

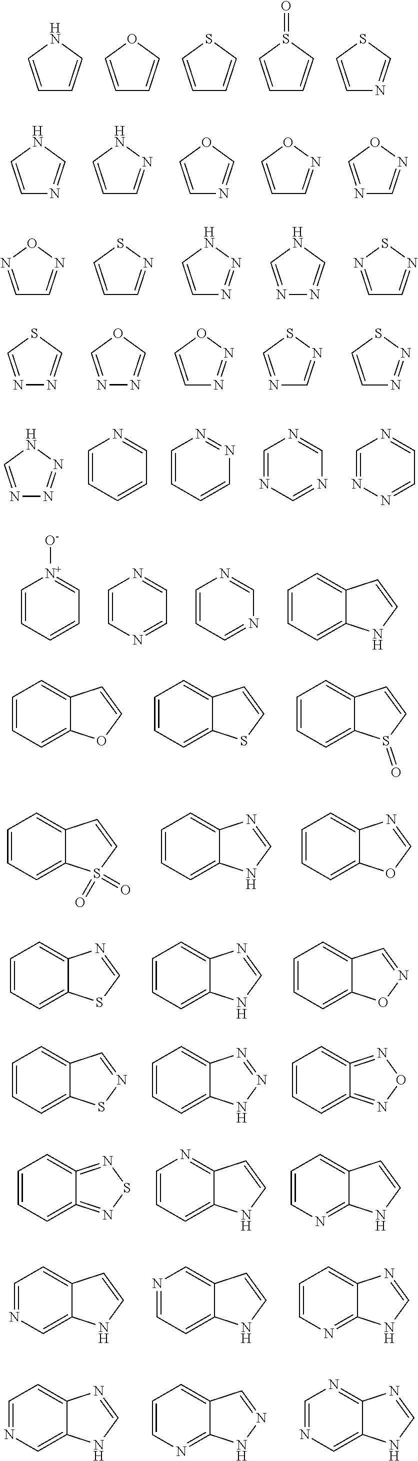

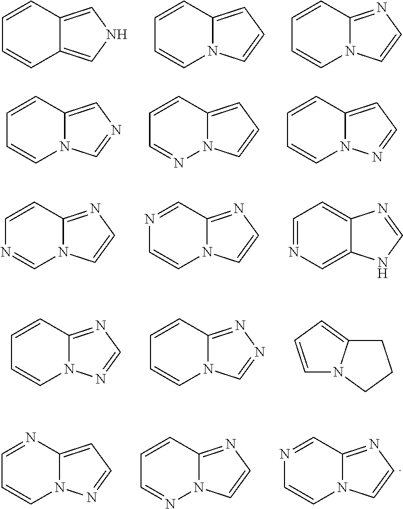

Many of the terms given above may be used repeatedly in the definition of a formula or group and in each case have one of the meanings given above, independently of one another.

The term, bicyclic ring systems” means groups consisting of 2 joined cyclic substructures including spirocyclic, fused, and bridged ring systems.

### PREFERRED EMBODIMENTS

In another embodiment the invention relates to compounds of formula (I)

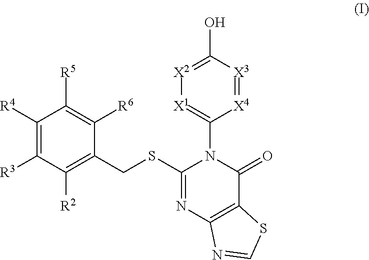

wherein at least one of X¹, X², X³, X⁴ is —N, provided that not more than two of X¹, X², X³, X⁴ are N simultaneously; and wherein R¹ is —H or -halogen; and wherein R¹¹ is selected from among a group consisting of —H, -halogen and —CN; and wherein R², R³, R⁴, R⁵, R⁶ are, independently of each other, selected from among a group consisting of —H, -halogen, —CF₃ and -methyl; or a salt thereof.

In another embodiment the invention relates to compounds of formula (I) wherein at least one of X¹, X², X³, X⁴ is —N, provided that not more than two of X¹, X², X³, X⁴ are N simultaneously; and wherein R¹ is —H or -halogen; and wherein R^{1.1} is selected from among a group consisting of —H, -halogen and —CN; and wherein R², R³, R⁴, R⁵, R⁶ are, independently of each other, selected from among a group consisting of —H, -halogen, —CF₃ and -methyl; or a salt thereof.

In another embodiment the invention relates to compounds of formula (I) wherein at least one of X¹, X², X³, X⁴ is —N, provided that not more than two of X¹, X², X³, X⁴ are N simultaneously; and wherein R¹ is —H or —F; and wherein R¹¹ is selected from among a group consisting of —H, —F and —CN; and wherein R², R³, R⁴, R⁵, R⁶ are, independently of each other, selected from among a group consisting of —H, —F, —Cl, —Br, —CF₃ and -methyl; or a salt thereof.

In another embodiment the invention relates to compounds of formula (I) wherein at least one of X¹, X², X³, X⁴ is —N, provided that not more than two of X¹, X², X³, X⁴ are N simultaneously; and wherein R¹ is —H; and wherein R^{1.1} is —F; and wherein R², R³, R⁴, R⁵, R⁶ are, independently of each other, selected from among a group consisting of —H, —F, —Cl, —Br, —CF₃ and -methyl; or a salt thereof.

In another embodiment the invention relates to compounds of formula (I) wherein at least one of X¹, X², X³, X⁴ is —N, provided that not more than two of X¹, X², X³, X⁴ are N simultaneously; and wherein R¹ is —H; and wherein R^{1.1} is —F; and wherein R², R³, R⁴, R⁵, R⁶ are, independently of each other, —H or —F; or a salt thereof.

In another embodiment the invention relates to compounds of formula (I) wherein X² is N, X¹ and X⁴ are C—R¹; wherein X³ is C—R^{1.1}; and wherein R¹ is selected from among a group consisting of —H, —F, —Cl; and wherein R^{1.1} is selected from among a group consisting of —H, -halogen and —CN; and wherein R², R³, R⁴, R⁵, R⁶ are, independently of each other, selected from among a group consisting of —H, -halogen, —CF₃ and -methyl; or a salt thereof.

In another embodiment the invention relates to compounds of formula (I) wherein X² is N, X¹ and X⁴ are C—R¹; wherein X³ is C—R^{1.1}; and wherein R¹ is selected from among a group consisting of —H, —F, —Cl; and wherein R^{1.1} is selected from among a group consisting of —H, -halogen and —CN; and wherein R², R³, R⁴, R⁵, R⁶ are, independently of each other, selected from among a group consisting of —H, -halogen, —CF₃ and -methyl; or a salt thereof.

In another embodiment the invention relates to compounds of formula (I) wherein X² is N, X¹ and X⁴ are C—R¹; wherein X³ is C—R^{1.1}; and wherein R¹ is —H or —F; and wherein R^{1.1} is —F; and wherein R², R³, R⁴, R⁵, R⁶ are, independently of each other, selected from among a group consisting of —H, —F, —Cl, —Br, —CF₃ and -methyl; or a salt thereof.

In another embodiment the invention relates to compounds of formula (I) wherein X² is N, X¹ and X⁴ are C—R¹; wherein X³ is C—R^{1.1}; and wherein R¹ is —H or —F; and wherein R^{1.1} is —F; and wherein R³ and R⁵ are —H; and wherein R², R⁴ and R⁶ are, independently of each other, selected from among a group consisting of —H, -halogen, —CF₃ and -methyl; or a salt thereof.

In another embodiment the invention relates to compounds of formula (I) wherein X² is N, X¹ and X⁴ are C—R¹; wherein X³ is C—R^{1.1}; and wherein R¹ is —H or —F; and wherein R^{1.1} is —F; and wherein R³ and R⁵ are —H; and wherein R², R⁴ and R⁶ are, independently of each other, selected from among a group consisting of —H, —F, —Cl, —Br, —CF₃ and -methyl; or a salt thereof.

In another embodiment the invention relates to compounds of formula (I) wherein X² is N, X¹ and X⁴ are C—R¹; wherein X³ is C—R^{1.1}; and wherein R¹ is —H; and wherein R^{1.1} is —F; and wherein R³ and R⁵ are —H; and wherein R², R⁴ and R⁶ are, independently of each other, selected from among a group consisting of —H, —F, —Cl, —Br, —CF₃ and -methyl; or a salt thereof.

In another embodiment the invention relates to compounds of formula (I) wherein X² is N, X¹ and X⁴ are C—R¹; wherein X³ is C—R^{1.1}; and wherein R¹ is —H; and wherein R^{1.1} is —F; and wherein R³ and R⁵ are —H; and wherein R², R⁴ and R⁶ are, independently of each other, —H or —F; or a salt thereof.

In another embodiment the invention relates to compounds of formula (I) wherein X¹, X², and X⁴ are C—R¹; wherein X³ is C—R^{1.1}; and wherein R¹ is selected from among a group consisting of —H, —F, —Cl; and wherein R^{1.1} is selected from among a group consisting of —H, -halogen and —CN; and wherein R², R³, R⁴, R⁵ and R⁶ are, independently of each other, selected from among a group consisting of —H, -halogen, —CF₃ and -methyl; or a salt thereof. In another embodiment the invention relates to compounds of formula (I) wherein X¹, X², and X⁴ are C—R¹; wherein X³ is C—R^{1.1}; and wherein R¹ is selected from among a group consisting of —H, —F, —Cl; and wherein R^{1.1} is selected from among a group consisting of —H, -halogen and —CN; and wherein R², R³, R⁴, R⁵, R⁶ are, independently of each other, selected from among a group consisting of —H, -halogen, —CF₃ and -methyl; or a salt thereof. In another embodiment the invention relates to compounds of formula (I) wherein X¹, X², and X⁴ are C—R¹; wherein X³ is C—R^{1.1}; and wherein R¹ is —H or —F; and wherein R^{1.1} is —F; and wherein R², R³, R⁴, R⁵, R⁶ are, independently of each other, selected from among a group consisting of —H, —Cl, —Br, —F, —CF₃ and -methyl; or a salt thereof.

In another embodiment the invention relates to compounds of formula (I) wherein X¹, X², and X⁴ are C—R¹; wherein X³ is C—R^{1.1}; and wherein R¹ is —H or —F; and wherein R^{1.1} is —F; and wherein R³ and R⁵ are —H; and wherein R², R⁴ and R⁶ are, independently of each other, selected from among a group consisting of —H, -halogen, —CF₃ and -methyl; or a salt thereof.

In another embodiment the invention relates to compounds of formula (I) wherein X¹, X², and X⁴ are C—R¹; wherein X³ is C—R^{1.1}; and wherein R¹ is —H or —F; and wherein R^{1.1} is —F; and wherein R³ and R⁵ are —H; and wherein R², R⁴ and R⁶ are, independently of each other, selected from among a group consisting of —H, —Cl, —Br, —F, —CF₃ and -methyl; or a salt thereof.

In another embodiment the invention relates to compounds of formula (I) wherein X¹, X², and X⁴ are C—R¹; wherein X³ is C—R^{1.1}; and wherein R¹ is —H; and wherein R^{1.1} is —F; and wherein R³ and R⁵ are —H; and wherein R², R⁴ and R⁶ are, independently of each other, selected from among a group consisting of —H, —F, —CF₃ and -methyl; or a salt thereof.

In another embodiment the invention relates to compounds of formula (I) wherein X¹, X², and X⁴ are C—R¹; wherein X³ is C—R^{1.11}; and wherein R¹ is —H; and wherein R^{1.1} is —F; and wherein R³ and R⁵ are —H; and wherein R², R⁴ and R⁶ are —H or —F; or a salt thereof.

In another embodiment the invention relates to compounds of formula (I) wherein X² and X³ are N; and wherein X¹ and X⁴ are C—R¹; and wherein R¹ is selected from among a group consisting of —H, —F, —Cl; and wherein R², R³, R⁴, R⁵, R⁶ are, independently of each other, selected from among a group consisting of —H, -halogen, —CF₃ and -methyl; or a salt thereof.

In another embodiment the invention relates to compounds of formula (I) wherein X² and X³ are N; and wherein X¹ and X⁴ are C—R¹; and wherein R¹ is selected from among a group consisting of —H, —F, —Cl; and wherein R², R³, R⁴, R⁵, R⁶ are, independently of each other, selected from among a group consisting of —H, -halogen, —CF₃ and -methyl; or a salt thereof.

In another embodiment the invention relates to compounds of formula (I) wherein X² and X³ are N; and wherein X¹ and X⁴ are C—R¹; and wherein R¹ is selected from among a group consisting of —H, —F, —Cl; and wherein R², R³, R⁴, R⁵, R⁶ are, independently of each other, selected from among a group consisting of —H, —Cl, —Br, —F, —CF₃ and -methyl; or a salt thereof.

In another embodiment the invention relates to compounds of formula (I) wherein X² and X³ are N; and wherein X¹ and X⁴ are C—R¹; and wherein R¹ is —H or —F; and wherein R³ and R⁵ are —H; and wherein R², R⁴ and R⁶ are, independently of each other, selected from among a group consisting of —H, -halogen, —CF₃ and -methyl; or a salt thereof.

In another embodiment the invention relates to compounds of formula (I) wherein X² and X³ are N; and wherein X¹ and X⁴ are C—R¹; and wherein R¹ is —H or —F; and wherein R³ and R⁵ are —H; and wherein R², R⁴ and R⁶ are, independently of each other, selected from among a group consisting of —H, —Cl, —Br, —F, —CF₃ and -methyl; or a salt thereof.

In another embodiment the invention relates to compounds of formula (I) wherein X² and X³ are N; and wherein X¹ and X⁴ are C—R¹; and wherein R¹ is —H or —F; and wherein R³ and R⁵ are —H; and wherein R², R⁴ and R⁶ are, independently of each other, —H, or —F; or a salt thereof.

In another embodiment the invention relates to compounds of formula (I) wherein X² and X³ are N; and wherein X¹ and X⁴ are C—R¹; and wherein R¹ is —H; and wherein R³ and R⁵ are —H; and wherein R², R⁴ and R⁶ are, independently of each other, selected from among a group consisting of —H, —F, —CF₃ and -methyl; or a salt thereof.

In another embodiment the invention relates to compounds of formula (I) wherein X² and X³ are N; and wherein X¹ and X⁴ are C—R¹; and wherein R¹ is —H; and wherein R³ and R⁵ are —H; and wherein R², R⁴ and R⁶ are, independently of each other, —H or —F; or a salt thereof.

In another embodiment the invention relates to compounds of formula (I) wherein X¹ and X are N; and wherein X³ is C—R^{1.1} and X⁴ is C—R¹; and wherein R¹ is selected from among a group consisting of —H, —F, —Cl; and wherein R^{1.1} is selected from among a group consisting of —H, -halogen and —CN; and wherein R², R³, R⁴, R⁵, R⁶ are, independently of each other, selected from among a group consisting of —H, -halogen, —CF₃ and -methyl; or a salt thereof.

In another embodiment the invention relates to compounds of formula (I) wherein X¹ and X² are N; and wherein X³ is C—R^{1.1} and X⁴ is C—R¹; and wherein R¹ is selected from among a group consisting of —H, —F, —Cl; and wherein R^{1.1} is selected from among a group consisting of —H, -halogen and —CN; and wherein R², R³, R⁴, R⁵, R⁶ are, independently of each other, selected from among a group consisting of —H, -halogen, —CF₃ and -methyl; or a salt thereof.

In another embodiment the invention relates to compounds of formula (I) wherein X¹ and X² are N; and wherein X³ is C—R^{1.1} and X⁴ is C—R¹; and wherein R¹ is —H or —F; and wherein R^{1.1} is —F; and wherein R², R³, R⁴, R⁵, R⁶ are, independently of each other, selected from among a group consisting of —H, —F, —CF₃ and -methyl; or a salt thereof. In another embodiment the invention relates to compounds of formula (I) wherein X¹ and X² are N; and wherein X³ is C—R^{1.1} and X⁴ is C—R¹; and wherein R¹ is —H or —F; and wherein R^{1.1} is —F; and wherein R³ and R⁵ are —H; and wherein R², R⁴ and R⁶ are, independently of each other, selected from among a group consisting of —H, -halogen, —CF₃ and -methyl; or a salt thereof.

In another embodiment the invention relates to compounds of formula (I) wherein X¹ and X² are N; and wherein X³ is C—R^{1.1} and X⁴ is C—R¹; and wherein R¹ is —H or —F; and wherein R^{1.1} is —F; and wherein R³ and R⁵ are —H; and wherein R², R⁴ and R⁶ are, independently of each other, selected from among a group consisting of —H, —Cl, —Br, —F, —CF₃ and -methyl; or a salt thereof.

In another embodiment the invention relates to compounds of formula (I) wherein X¹ and X² are N; and wherein X³ is C—R^{1.1} and X⁴ is C—R¹; and wherein R¹ is —H; and wherein R^{1.1} is —F; and wherein R³ and R⁵ are —H; and wherein R², R⁴ and R⁶ are, independently of each other, selected from among a group consisting of —H, —F, —CF₃ and -methyl; or a salt thereof. In another embodiment the invention relates to compounds of formula (I) wherein X¹ and X² are N; and wherein X³ is C—R^{1.1} and X⁴ is C—R¹; and wherein R¹ is —H; and wherein R^{1.1} is —F; and wherein R³ and R⁵ are —H; and wherein R², R⁴ and R⁶ are, independently of each other, —H, or —F; or a salt thereof.

In another embodiment the invention relates to compounds of formula (I) wherein X¹ and X³ are N; and wherein X² and X⁴ are C—R¹; X³ is N and X⁴ is C—R¹; and wherein R¹ is —H; and R³ and R⁵ are —H; and wherein R², R⁴ and R⁶ are, independently of each other, —H, or —F; or a salt thereof.

In another embodiment the invention relates to compounds selected from among the group consisting of

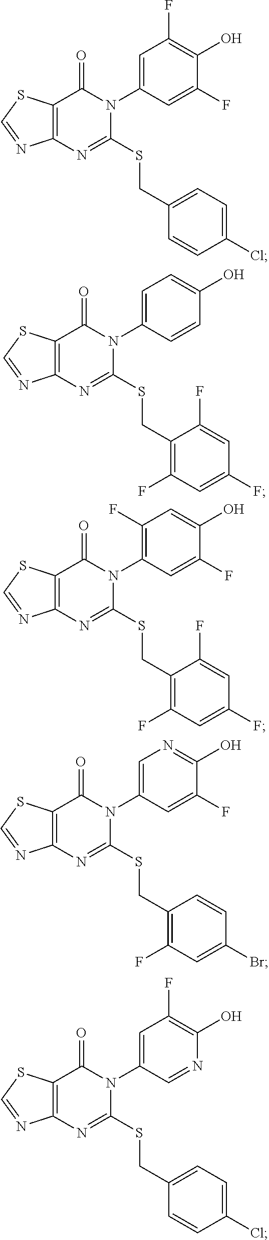

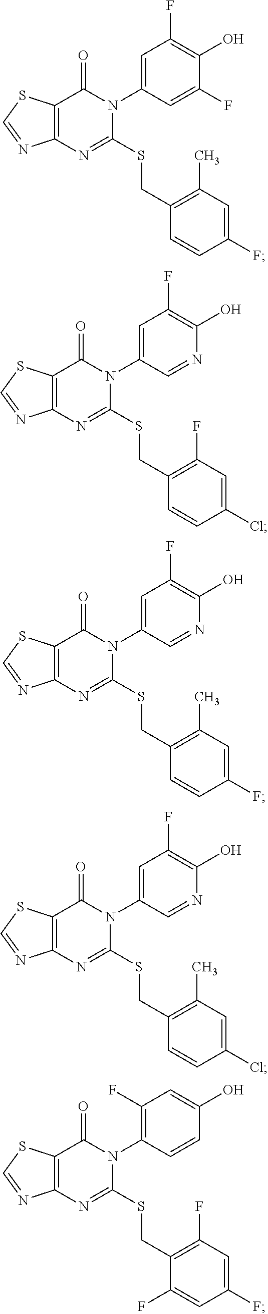

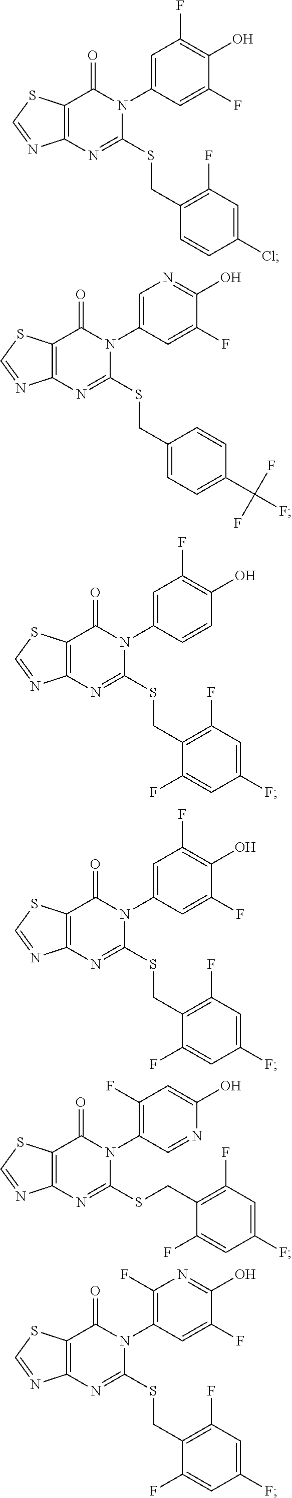

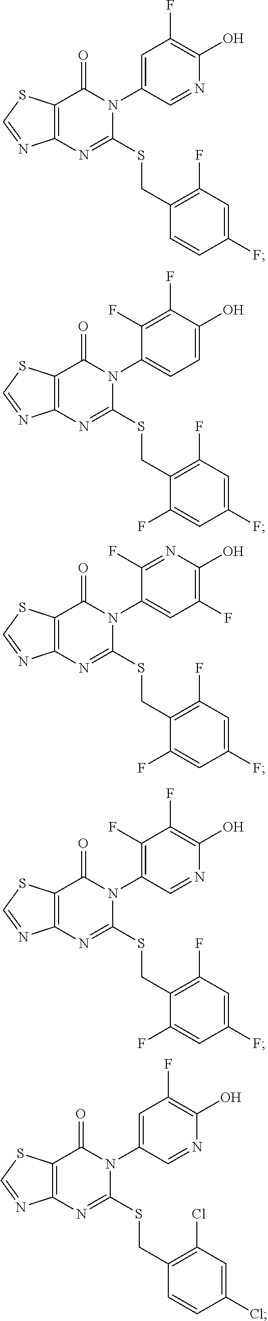

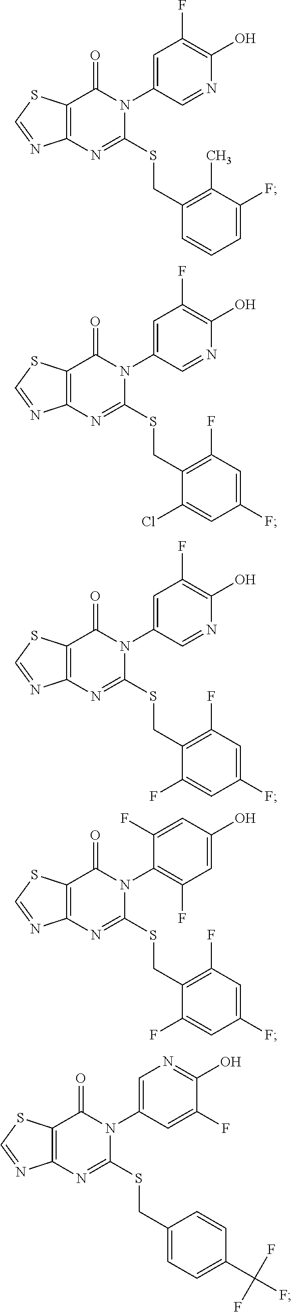

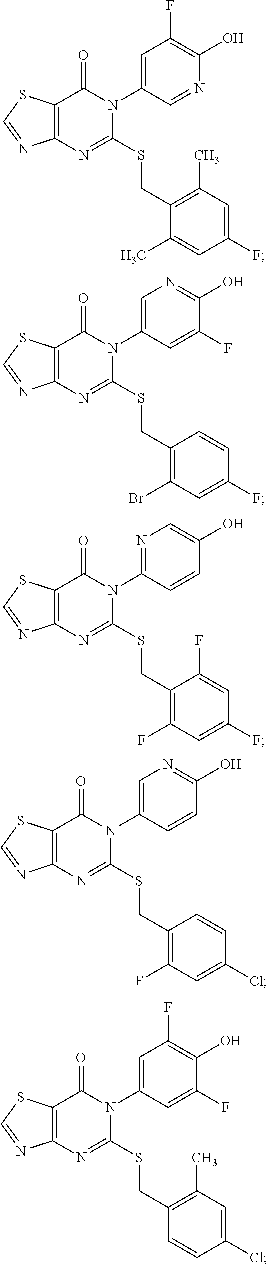

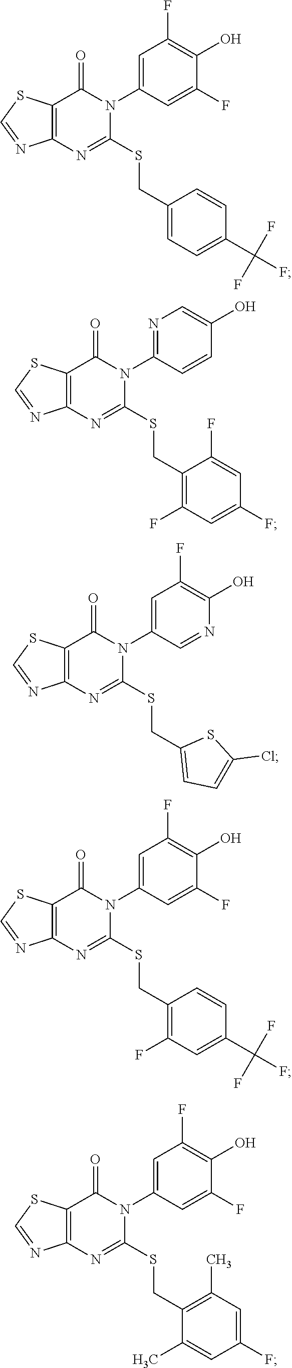

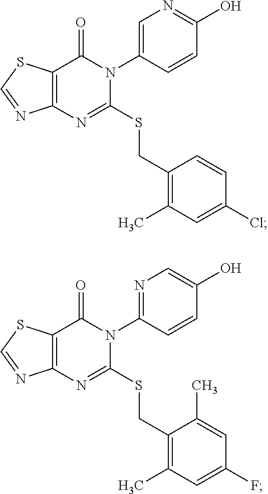 [SMILES: Cc1cc(Cl)ccc1CSc1nc2ncsc2c(=O)n1-c1ccc(O)nc1 ; Cc1cc(F)cc(C)c1CSc1nc2ncsc2c(=O)n1-c1ccc(O)cn1]

or a pharmaceutically acceptable salt thereof.

In another embodiment the invention relates to compounds selected from among the group consisting of

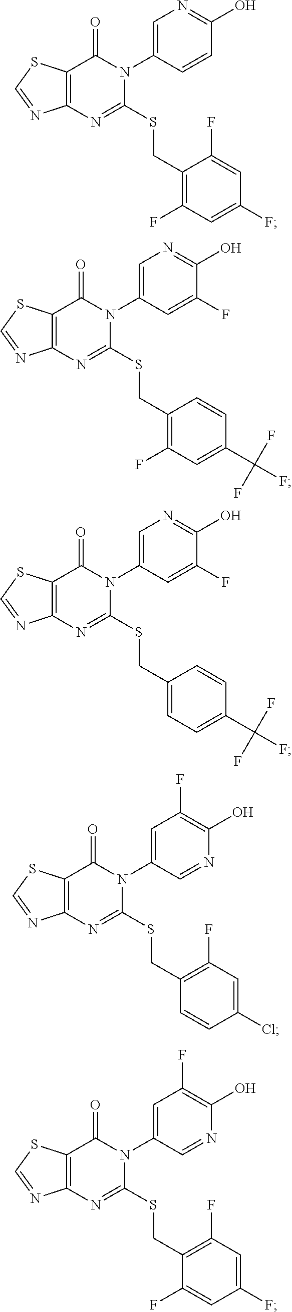

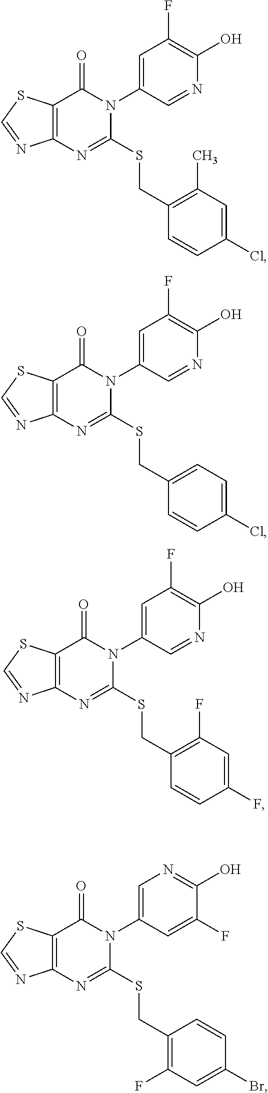

or a pharmaceutically acceptable salt thereof.

In another embodiment the invention relates to compounds selected from among the group consisting of

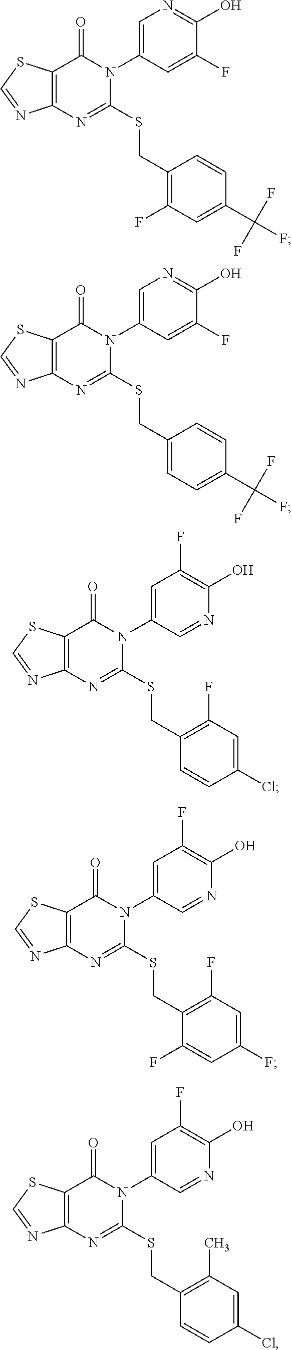

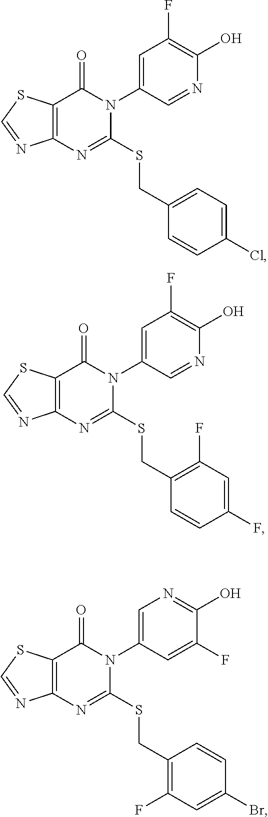

or a pharmaceutically acceptable salt thereof.

### Biological Assays

### Assay to Determine Inhibition of NOX4

### Cell Line Generation for NOX4 Inhibition Assay

The synthetic gene phNOX4_DNA3_1_Zeo (Accession: AAF68973) is assembled from synthetic oligonucleotides and/or PCR products. The fragment is cloned into pcDNA3.1_Zeo_A011 using NheI and XhoI cloning sites. The plasmid DNA is purified from transformed bacteria and its concentration is determined by UV spectroscopy. The final construct is verified by sequencing. The plasmid is transfected via electroporation (Amaxa electroporation device) in combination with Nucleofector Kit V. Selection of NOX4 overexpressing genes is achieved using selection antibiotic Zeocin.

### NOX4 Inhibition Assay (HyPerBlu™)

The inhibitory activity of the example compounds of the invention is determined using the following procedure:

NOX4 inhibition is assessed utilizing HEK293 cells stably overexpressing human NOX4 (hNOX4), generating constitutively high levels of hydrogen peroxide (H₂O₂). Cells are cultured in the Dulbecco's Modified Eagle Medium (DMEM) media containing 4.5 g/L glucose supplemented with 10% fetal calf serum and 250 μg/ml Zeocin in an incubator at 37° C. with 5% CO₂. For the assay, cells are seeded in 384 well plates. After 24 h cells are washed and treated with several concentrations (10 nM-30 μM) of the test compounds (diluted in DMSO) or 100 μM diphenyleneiodonium chloride (DPI, used as positive control) in assay buffer consisting of phosphate-buffered saline (PBS) with 4-(2-hydroxyethyl)-1-piperazineethanesulfonic acid (HEPES), and incubated for 2 hours at 24° C. in a humidified incubator.

After incubation Lumigen HyPerBlu™ is added and cells are incubated for additional 30 min at 24° C. in a humidified incubator. Next, luminescence is measured using an Envision Multimode Plate reader to determine levels of produced H₂O₂ in the wells. Results are visible in Table 1.

TABLE 1

| Example-Number | IC₅₀ (NOX4) in nM |
|---|---|
| 1 | 555 |
| 2 | 39 |
| 3 | 73 |
| 4 | 91 |
| 5 | 24 |
| 6 | 87 |
| 7 | 77 |
| 8 | 43 |
| 9 | 29 |
| 10 | 27 |
| 11 | 42 |
| 12 | 51 |
| 13 | 61 |
| 14 | 38 |
| 15 | 33 |
| 16 | 367 |
| 17 | 314 |
| 18 | 117 |
| 19 | 80 |
| 20 | 127 |
| 21 | 174 |
| 22 | 69 |
| 23 | 96 |
| 24 | 98 |
| 25 | 101 |
| 26 | 120 |
| 27 | 135 |
| 28 | 136 |
| 29 | 145 |
| 30 | 539 |
| 31 | 60 |
| 32 | 40 |
| 33 | 29 |
| 34 | 29 |
| 35 | 33 |
| 36 | 34 |
| 37 | 34 |
| 38 | 48 |
| 39 | 56 |
| 40 | 59 |
| 41 | 60 |
| 42 | 62 |
| 43 | 63 |
| 44 | 63 |
| 45 | 113 |
| 46 | 130 |
| 47 | 426 |
| 48 | 201 |
| 49 | 45 |
| 50 | 47 |
| 51 | 51 |
| 52 | 52 |
| 53 | 116 |
| 54 | 111 |
| 55 | 116 |
| 56 | 153 |
| 57 | 124 |
| 58 | 140 |
| 59 | 206 |
| 60 | 237 |
| 61 | 103 |
| 62 | 127 |
| 63 | 126 |
| 64 | 133 |
| 65 | 137 |
| 66 | 137 |
| 67 | 165 |
| 68 | 143 |
| 69 | 65 |

### Assays to Determine the Selectivity Against NOX1, NOX2, NOX3, NOX5

### Cell Line Generation

Plasmids are Purchased from Vectorbuilder.
for HEK-hNOX1 all necessary subunits are cloned accordingly:
   pLV-CMV-hNOX1-puro (NM_007052.5)
   pLV-CMV-hNOXO1-blas (NM_172168.3)
   pLV-CMV-hp22phox-hygro (NM_000101.4)
   pLV-CMV-hNOXA1-neo (NM_006647.2)
for HEK-hNOX3
   pLV-CMV-hNOX3-puro (NM_015718.3)

3rd generation lentiviral particles are produced in HEK293 suspension cells by transient transfection of three helper and expression plasmids. Crucial lentivirus supernatants are harvested, clarified by filtration and concentrated by precipitation. Lentivirus titers are determined in transducing units in (TU/mL) by transduction of HT1080 cells and colony forming assay. Parental HEK293 cells, purchased from CLS GmbH, are transduced with replication-incompetent 3rd generation lentiviral particles (MOI 2) and expanded for at least 2 weeks before cryopreservation of tested lots. During cultivation, medium is changed three times per week and cells are sub-cultured at least once per week. Selection antibiotics are added according to the plasmids (1 μg/ml puromycin, 1.5 μg/ml blasticidin, 200 μg/ml G418 and 100 μg/ml hygromycin).

### For HEK-hNOX5

The synthetic gene hNOX5 (Accession: Q96PH1) is cloned into pcDNA3.1_Zeo. The plasmid DNA is purified from transformed bacteria and its concentration is determined by UV spectroscopy. The final construct is verified by sequencing. The plasmid is transfected via electroporation (Amaxa electroporation device) in combination with Nucleofector Kit V. Selection of NOX5 overexpressing genes is achieved using selection antibiotic Zeocin

### NOX1 Inhibition Assay (L-012)

Evaluation of the NOX4 inhibitors on NOX1 activity is assessed in HEK293 cells stably overexpressing human NOX1 (hNOX1).

Cells are cultured in the DMEM media containing 4.5 g/L glucose supplemented with 10% fetal calf serum (FCS), Puromycin 1 μg/mL, Blasticidin 1.5 μg/mL, Geneticin (G418) 200 μg/mL and Hygromycin 100 μg/mL in an incubator at 37° C. with 5% CO₂. For the assay cells are seeded in 384 well plates in DMEM media with 10% FCS. After 24 h, cells are washed, treated for 30 mins with several concentrations (30 nM-100 μM) of test compounds and then stimulated with phorbol myristate acetate (PMA) at 1 μM to induce NOX1-dependent ROS production (except wells used as a positive control). Lastly, 8-amino-5-chloro-2,3-dihydro-7-phenyl-pyrido[3,4-d]pyridazine-1,4-dione sodium salt (L-012, CAS 143556-24-5) is added at 400 μM to all wells, and cells are incubated in an incubator for an additional 3 h. Afterwards luminescence is measured with a SpectraMax Paradigm Microplate Reader. Data can be found in table 2.

### NOX2 Inhibition Assay (L-012)

Evaluation of the NOX4 inhibitors on NOX2 activity is assessed in human blood-derived granulocytes.

Different concentrations of the test compounds (30 nM-100 μM), DMSO (as a negative control) or diphenyleneidonium chloride (DPI; 30 μM, as a positive control), prepared in assay buffer (PBS containing CaCl2 and MgCl2+0.1% bovine serum albumin (BSA)), are placed in 384 well plates. Afterwards, freshly isolated granulocytes are added to the wells containing the compounds in assay buffer and incubated for 1 h at 37° C. Next, a solution of Nformylmethionine-leucyl-phenylalanine (fMLP; 730 nM), phorbol myristate acetate (PMA, 100 nM) (stimuli of ROS production) and L-012 (200 μM, ROS indicator) is added to the wells and the cells are incubated for additional 30 mins at 37° C. Afterwards luminescence is measured in a SpectraMax M5 microplate reader. Data can be found in table 2.

### NOX3 Inhibition Assay (L-012)

Evaluation of the NOX4 inhibitors on NOX3 activity is assessed in HEK293 cells stably overexpressing human NOX3 (hNOX3).

Cells are cultured in the DMEM media containing 4.5 g/L glucose supplemented with 10% fetal calf serum (FCS), Puromycin 1 μg/mL and Blasticidin 1.5 μg/mL in an incubator at 37° C. with 5% CO₂. Cells are seeded in 384 well plates for 2 days. For the assay cells are washed and treated with several concentrations (10 nM-30 μM) of test compound (diluted in DMSO) or 100 μM DPI (as a positive control in assay buffer consisting of PBS), and incubated for 2 hours at 24° C. in a humidified incubator. Cells are then stimulated with PMA at 0.1 μM to induce NOX3-dependent ROS production. Finally, L-012 is added at 100 μM and incubated for 2 hours at 24° C. in a humidified incubator. Luminescence is measured with an PHERAstar multimode reader. Data can be found in table 2.

### NOX5 Inhibition Assay (L-012)

Evaluation of the NOX4 inhibitors on NOX5 activity is assessed in HEK293 cells stably overexpressing human NOX5 (hNOX5).

Cells are cultured in the DMEM media containing 4.5 g/L glucose supplemented with 10% fetal calf serum and 250 ag/ml Zeocin in an incubator at 37° C. with 5% CO₂. Cells are seeded in 384 well plates over-night. For the assay cells are washed and treated with several concentrations (10 nM-30 μM) of test compounds (diluted in DMOX and added to assay buffer consisting of PBS) or 100 μM DPI as a positive control and incubated for 2 hours at 24° C. in a humidified incubator. Cells are then stimulated with PMA at 0.1 μM to induce NOX5-dependent ROS production. Finally, L-012 is added at 100 μM and incubated for 2 hours at 24° C. in a humidified incubator. Luminescence is measured with an PHERAstar multimode reader. Data can be found in table 2.

TABLE 2

| Example | IC₅₀ (NOX1) | IC₅₀ (NOX2) | IC₅₀ (NOX3) | IC₅₀ (NOX5) |
|---|---|---|---|---|
| Number | in μM | in uM | in μM | in μM |
| 1 | >100 | N/A | N/A | N/A |
| 2 | N/A | >100 | 4.1 | >100 |
| 3 | 1.7 | >100 | >100 | >100 |
| 4 | N/A | >100 | 4.1 | >100 |
| 5 | 0.3 | >100 | >100 | >100 |
| 6 | >100 | >100 | >100 | >100 |
| 7 | >100 | >100 | >100 | >100 |
| 8 | 1.1 | >30 | 2.7 | >100 |
| 9 | 1.9 | >100 | 3.7 | >100 |
| 10 | 0.5 | >30 | 4.0 | 11.5 |
| 11 | 0.9 | >30 | 16.3 | 13.0 |
| 12 | 2.9 | >30 | 7.6 | 6.6 |
| 13 | 3.2 | 84.6 | 16.0 | 5.1 |
| 14 | 2.3 | 13.4 | 25.5 | 10.2 |
| 15 | 4.1 | 10.3 | >100 | 59.0 |
| 16 | 15.0 | >100 | 59.1 | 36.0 |
| 17 | 14.4 | N/A | 61.4 | 17.5 |
| 18 | 0.06 | >30 | 0.4 | >100 |
| 19 | 16.9 | 48.1 | >100 | >100 |
| 20 | 7.0 | >100 | 8.1 | 32.7 |
| 21 | >100 | >100 | >100 | >100 |
| 22 | >100 | >30 | >100 | >100 |
| 23 | 48.1 | >100 | >100 | >100 |
| 24 | 20.6 | 10.3 | >100 | >100 |
| 25 | >100 | >100 | >100 | >100 |
| 26 | 33.0 | >100 | 62.1 | 57.4 |
| 27 | 76.0 | >100 | 86.8 | 93.3 |
| 28 | 27.1 | >30 | >100 | >100 |
| 29 | 33.3 | 36.9 | >100 | >100 |
| 30 | 37.8 | >30 | 98.1 | 94.8 |
| 31 | 22.9 | 10.5 | >100 | >100 |
| 32 | >100 | >100 | 38.0 | 55.7 |
| 33 | 35.5 | >100 | >100 | >100 |
| 34 | 14.7 | >100 | 63.6 | 46.5 |
| 35 | >100 | >100 | 39.9 | 65.5 |
| 36 | >100 | >100 | 87.9 | >100 |
| 37 | 18.8 | >100 | 70.4 | 64.5 |
| 38 | >100 | >100 | >100 | >100 |
| 39 | 24.5 | >100 | 56.2 | 47.9 |
| 40 | 32.9 | >100 | 39.2 | 56.3 |
| 41 | 9.8 | 39.3 | 32.3 | 35.8 |
| 42 | 5.6 | >100 | 46.4 | 46.8 |
| 43 | >100 | >100 | >100 | >100 |
| 44 | 43.2 | >100 | 73.1 | 68.1 |
| 45 | 30.9 | >100 | 71.5 | 79.0 |
| 46 | 48.4 | >100 | 56.4 | 44.7 |
| 47 | 4.9 | >30 | 18.8 | >100 |
| 48 | 36.7 | >100 | >100 | >100 |
| 49 | >100 | >100 | >100 | >100 |
| 50 | 27.6 | >100 | 64.7 | 60.2 |
| 51 | 3.0 | >100 | 6.4 | 59.4 |
| 52 | 21.2 | >100 | 15.5 | 38.9 |
| 53 | >100 | >100 | >100 | >100 |
| 54 | 1.0 | >100 | 49.4 | 47.6 |
| 55 | 16.5 | >100 | >100 | 85.8 |
| 56 | 2.7 | >100 | >100 | >100 |
| 57 | N/A | >100 | >100 | >100 |
| 58 | 11.8 | >100 | >100 | >100 |
| 59 | 21.0 | >100 | >100 | >100 |
| 60 | >100 | >100 | >100 | >100 |
| 61 | >100 | >100 | 28.2 | >100 |
| 62 | >100 | >100 | >100 | >100 |
| 63 | 43.4 | 46,0 | >100 | >100 |
| 64 | >100 | >100 | >100 | >100 |
| 65 | >100 | >100 | 83.4 | >100 |
| 66 | >100 | >100 | >100 | 74.8 |
| 67 | >100 | >30 | >100 | >100 |
| 68 | 11.0 | >100 | >100 | >100 |
| 69 | >100 | >100 | 77.5 | >100 |

* N/A = not available

### Evaluation of Hepatocyte Clearance

The metabolic degradation of the test compound is assayed in a hepatocyte suspension. Hepatocytes (cryopreserved) are incubated in Dulbecco's modified eagle medium (supplemented with 3.5 ag glucagon/500 mL, 2.5 mg insulin/500 mL and 3.75 mg/500 mL hydrocortisone) containing 5% human serum.

Following a 30 min preincubation in an incubator (37° C., 10% CO₂) 5 μl of test compound solution (80 μM; from 2 mM in DMSO stock solution diluted 1:25 with medium) are added into 395 μl hepatocyte suspension (cell density in the range 0.25-5 Mio cells/mL, typically 1 Mio cells/mL; final concentration of test compound 1 μM, final DMSO concentration 0.05%).

The cells are incubated for six hours (incubator, orbital shaker) and samples (25 μl) are taken at 0, 0.5, 1, 2, 4 and 6 hours. Samples are transferred into acetonitrile and pelleted by centrifugation (5 min). The supernatant is transferred to a new 96-deepwell plate, evaporated under nitrogen and resuspended.

### Decline of Parent Compound is Analyzed by HPLC-MS/MS.

### CLᵢₙₜ is Calculated as Follows:

CL_INTRINSIC=Dose/AUC=(C0/CD)/(AUD+clast/k)×1000/60.
C0: initial concentration in the incubation [μM], CD: cell density of vital cells [10⁶ cells/mL], AUD: area under the data [μM×h], clast: concentration of last data point [μM],
k: slope of the regression line for parent decline [h-1].

The calculated in vitro hepatic intrinsic clearance can be scaled up to the intrinsic in vivo hepatic clearance and used to predict hepatic in vivo blood clearance (CL) by the use of a liver model (well stirred model).

CL_INTRINSIC_INVIVO[ml/min/kg]=(CL_INTRINSIC[μL/min/10⁶ cells]×hepatocellularity[10⁶ cells/g liver]×liver factor[g/kg bodyweight])/1000

CL[ml/min/kg]=CL_INTRINSIC_INVIVO[ml/min/kg]×hepatic blood flow[ml/min/kg]/(CL_INTRINSIC_INVIVO[ml/min/kg]+hepatic blood flow[ml/min/kg])

Qh[%]=CL[ml/min/kg]/hepatic blood flow[ml/min/kg])
Hepatocellularity, human: 120×10⁶ cells/g liver
Liver factor, human: 25.7 g/kg bodyweight
Blood flow, human: 21 ml/(min×kg)

### The Resulting Data are Visible in Table 3:

| Example Number | Qh [%] |
|---|---|
| 2 | 53 |
| 4 | 41 |
| 5 | 56 |
| 7 | 55 |
| 8 | 42 |
| 9 | 65 |
| 10 | 72 |
| 11 | 79 |
| 12 | 77 |
| 14 | 73 |
| 15 | 61 |
| 16 | 73 |
| 17 | 70 |
| 18 | 48 |
| 19 | 52 |
| 20 | 51 |
| 21 | 27 |
| 22 | 45 |
| 23 | 55 |
| 24 | 45 |
| 25 | 41 |
| 26 | 41 |
| 27 | 37 |
| 28 | 29 |
| 29 | 54 |
| 30 | 22 |
| 31 | 14 |
| 32 | 15 |
| 33 | 15 |
| 34 | 8 |
| 35 | 15 |
| 36 | 34 |
| 37 | 16 |
| 38 | 11 |
| 39 | 26 |
| 41 | 26 |
| 42 | 14 |
| 43 | 37 |
| 44 | 29 |
| 45 | 23 |
| 46 | 21 |
| 47 | 15 |
| 48 | 22 |
| 49 | 25 |
| 50 | 19 |
| 51 | 10 |
| 52 | 20 |
| 53 | 13 |
| 54 | 33 |
| 55 | 15 |
| 56 | <4 |
| 57 | 7 |
| 58 | 19 |
| 59 | <4 |
| 60 | 8 |
| 61 | 55 |
| 62 | 58 |
| 64 | 47 |
| 65 | 50 |
| 66 | 40 |
| 67 | 37 |
| 69 | <4 |

### Method of Treatment

The present invention is directed to compounds of general formula (I) which are useful in the prevention and/or treatment of a disease and/or condition associated with or modulated by NOX4 activity, including but not limited to the treatment and/or prevention of chronic liver diseases, portal hypertension, viral infections, cancer, interstitial lung diseases, retinopathies, acute and chronic inflammation as well as fibrotic diseases. Particularly, the pharmaceutical compositions of the invention are suitable for the therapy of interstitial lung diseases, e.g. idiopathic pulmonary disease, and may also be suitable for the therapy of fibrotic diseases, allergic and inflammatory diseases.

The compounds of general formula (I) are useful for the prevention and/or treatment of: vascular inflammation, atherosclerosis, interstitial lung diseases (e.g. idiopathic pulmonary fibrosis, progressive pulmonary fibrosis), liver fibrosis, pulmonary hypertension, portal hypertension, liver cirrhosis, acute on chronic liver failure (ACLF), sepsis, multi-organ failure, diabetic retinopathies, wet age-related macular degeneration (AMD), dry AMD, cardiovascular diseases, NOX4+ cancer associated fibroblast rich tumors (pancreatic, lung, breast, colon, head and neck tumors), systemic sclerosis, inflammatory bowel disease, Duchenne muscular dystrophy, COVID-19, acute respiratory distress syndrome, influenza.

Accordingly, the present invention relates to a compound of general formula (I) for use as a medicament.

Furthermore, the present invention relates to the use of a compound of general formula (I) for the treatment and/or prevention of a disease and/or condition associated with or modulated by NOX4 activity.

Furthermore, the present invention relates to the use of a compound of general formula (I) for the treatment and/or prevention of chronic liver diseases, viral infections, cancer, interstitial lung diseases, retinopathies, acute and chronic inflammation as well as fibrotic diseases.

Particularly, the pharmaceutical compositions of the invention are suitable for the therapy of interstitial lung diseases, e.g. idiopathic pulmonary disease, and may also be suitable for the therapy of fibrotic, allergic and inflammatory diseases.

Furthermore, the present invention relates to the use of a compound of general formula (I) for the treatment and/or prevention of: vascular inflammation, atherosclerosis, interstitial lung diseases, e.g. idiopathic pulmonary fibrosis, progressive pulmonary fibrosis, liver fibrosis, pulmonary hypertension, portal hypertension, liver cirrhosis, acute on chronic liver failure (ACLF), sepsis, multi-organ failure, diabetic retinopathies, wet age-related macular degeneration (AMD), dry AMD, cardiovascular diseases, NOX4+ cancer associated fibroblast rich tumors (pancreatic, lung, breast, colon, and head and neck tumors), systemic sclerosis, inflammatory bowel disease, Duchenne muscular dystrophy, COVID-19, acute respiratory distress syndrome, influenza, pulmonary hypertension.

In a further aspect the present invention relates to a compound of general formula (I) for use in the treatment and/or prevention of above mentioned diseases and conditions.

In a further aspect the present invention relates to the use of a compound of general formula (I) for the preparation of a medicament for the treatment and/or prevention of above mentioned diseases and conditions.

In a further aspect of the present invention the present invention relates to methods for the treatment or prevention of above mentioned diseases and conditions, which method comprises the administration of an effective amount of a compound of general formula (I) to a human being.

The dose range of the compounds of general formula (I) applicable per day is usually from 0.00001 to 100 mg per kg body weight, for example from 0.00001 to 10 mg per kg body weight of the patient. Each dosage unit may conveniently contain from 0.001 to 1000 mg, for example from 0.001 to 100 mg.

The actual pharmaceutically effective amount or therapeutic dosage will usually depend on factors known by those skilled in the art such as age and weight of the patient, route of administration and severity of disease. In any case the compounds will be administered at dosages and in a manner which allows a pharmaceutically effective amount to be delivered based upon patient's unique condition.

### Pharmaceutical Composition

Suitable preparations for administering the compounds of formula (I) will be apparent to those with ordinary skill in the art and include for example tablets, pills, capsules, suppositories, lozenges, troches, solutions, syrups, elixirs, sachets, injectables, inhalables and powders etc.

Suitable tablets may be obtained, for example, by mixing one or more compounds according to formula (I) with known excipients, for example inert diluents, carriers, disintegrants, adjuvants, surfactants, binders and/or lubricants.

### Combination Therapy

The compounds of the invention may further be combined with one or more, preferably one additional therapeutic agent. According to one embodiment the additional therapeutic agent is selected from the group of therapeutic agents useful in the treatment of diseases or conditions described hereinbefore, in particular associated with chronic liver diseases, viral infections, cancer, interstitial lung diseases, retinopathies, acute and chronic inflammation as well as fibrotic diseases.

According to another embodiment, the additional therapeutic agent is selected from the group of therapeutic agents useful in the treatment of diseases or conditions described hereinbefore, in particular associated with vascular inflammation, atherosclerosis, interstitial lung diseases (e.g. idiopathic pulmonary fibrosis, progressive pulmonary fibrosis), liver fibrosis, pulmonary hypertension, portal hypertension, liver cirrhosis, acute on chronic liver failure (ACLF), sepsis, multi-organ failure, diabetic retinopathies, wet age-related macular degeneration (AMD), dry AMD, cardiovascular diseases, NOX4+ cancer associated fibroblast rich tumors (pancreatic, lung, breast, colon, head and neck tumors), systemic sclerosis, inflammatory bowel disease, Duchenne muscular dystrophy, COVID-19, acute respiratory distress syndrome, influenza.

Additional therapeutic agents that are suitable for such combinations include in particular those, which, for example, potentiate the therapeutic effect of one or more active substances with respect to one of the indications mentioned and/or allow the dosage of one or more active substances to be reduced.

Therefore, a compound of the invention may be combined with one or more additional therapeutic agents selected from the group consisting of antifibrotics (e.g. Ofev, PDE4i); of immunotherapeutics (e.g. PD-1, aCTLA-4); of sGC activators; of ATX-inhibitors; of SGLT2 inhibitors (e.g. dapagliflozin, empagliflozin); of THRb inhibitors; of GLP1 agonists and GLP1 agonist combinations; of FGF-analogs, such as FGF21 or FGF19; of KRAS-G12C-inhibitors (e.g. sotorasib); of KRAS-G12D-inhibitors; of MDM2-p53-antagonists; of Her2-inhibitors; of platinum derivatives (e.g. cisplatin, oxaliplatin, carboplatin); of alkylation agents (e.g. estramustin, meclorethamine, melphalan, chlorambucil, busulphan, dacarbazin, cyclophosphamide, ifosfamide, temozolomide, nitrosoureas such as for example carmustin and lomustin, thiotepa); of antimitotic agents (e.g. Vinca alkaloids e.g. vinblastine, vindesin, vinorelbin and vincristine); of taxanes such as paclitaxel, docetaxel, nab-paclitaxel (Abraxane); of angiogenesis inhibitors (e.g. tasquinimod, bevacizumab); of tubuline inhibitors; of DNA synthesis inhibitors; of PARP inhibitors; of topoisomerase inhibitors (e.g. epipodophyllotoxins such as for example etoposide and etopophos, teniposide, amsacrin, topotecan, irinotecan, mitoxantrone); of antimetabolites (e.g. methotrexate, raltitrexed, 5-fluorouracil (5-FU), capecitabine, floxuridine, gemcitabine, mercaptopurine, thioguanine, cladribine, pentostatin, cytarabine (ara C), fludarabine, combination of trifluridine and tipiracil (=TAS102)); of antitumor antibiotics (e.g. anthracyclins such as doxorubicin, doxil (pegylated liposomal doxorubicin hydrochloride), myocet (non-pegylated liposomal doxorubicin), daunorubicin, epirubicin and idarubicin, mitomycin-C, bleomycin, dactinomycin, plicamycin, streptozocin); of inhibitors of vascular endothelial growth factor. Furthermore, the compounds according to the present invention can be combined with a radiotherapy regime.

Therefore, in another aspect, this invention relates to the use of a compound according to the invention in combination with one or more additional therapeutic agents described hereinbefore and hereinafter for the treatment of diseases or conditions which may be affected or which are mediated by NOX4, in particular diseases or conditions as described hereinbefore and hereinafter.

In a further aspect this invention relates to a method for treating a disease or condition which can be influenced by the inhibition of NOX4 in a patient that includes the step of administering to the patient in need of such treatment a therapeutically effective amount of a compound of formula (I) or a pharmaceutically acceptable salt thereof in combination with a therapeutically effective amount of one or more additional therapeutic agents.

In a further aspect this invention relates to the use of a compound of formula (I) or a pharmaceutically acceptable salt thereof in combination with one or more additional therapeutic agents for the treatment of diseases or conditions which can be influenced by the inhibition of NOX4 in a patient in need thereof.

In yet another aspect the present invention relates to a method for the treatment of a disease or condition mediated by NOX4 activity in a patient that includes the step of administering to the human patient, in need of such treatment a therapeutically effective amount of a compound of the present invention in combination with a therapeutically effective amount of one or more additional therapeutic agents described in hereinbefore and hereinafter.

The use of the compound according to the invention in combination with the additional therapeutic agent may take place simultaneously or at staggered times.

The compound according to the invention and the one or more additional therapeutic agents may both be present together in one formulation, for example a tablet or capsule, or separately in two identical or different formulations, for example as a so-called kit-of-parts.

Consequently, in another aspect, this invention relates to a pharmaceutical composition that comprises a compound according to the invention and one or more additional therapeutic agents described hereinbefore and hereinafter, optionally together with one or more inert carriers and/or diluents.

Other features and advantages of the present invention will become apparent from the following more detailed examples which illustrate, by way of example, the principles of the invention.

### Preparation

The compounds according to the present invention and their intermediates may be obtained using methods of synthesis which are known to the one skilled in the art and described in the literature of organic synthesis. Preferably, the compounds are obtained in analogous fashion to the methods of preparation explained more fully hereinafter, in particular as described in the experimental section. In some cases, the order in carrying out the reaction steps may be varied. Variants of the reaction methods that are known to the one skilled in the art but not described in detail here may also be used.

The general processes for preparing the compounds according to the invention will become apparent to the one skilled in the art studying the following schemes. Any functional groups in the starting materials or intermediates may be protected using conventional protecting groups. These protecting groups may be cleaved again at a suitable stage within the reaction sequence using methods familiar to the one skilled in the art.

The compounds according to the invention are prepared by the methods of synthesis described hereinafter in which the substituents of the general formulae have the meanings given herein before. These methods are intended as an illustration of the invention without restricting its subject matter and the scope of the compounds claimed to these examples. Where the preparation of starting compounds is not described, they are commercially obtainable or may be prepared analogously to known compounds or methods described herein. Substances described in the literature are prepared according to the published methods of synthesis. Abbreviations are as defined in the Examples section.

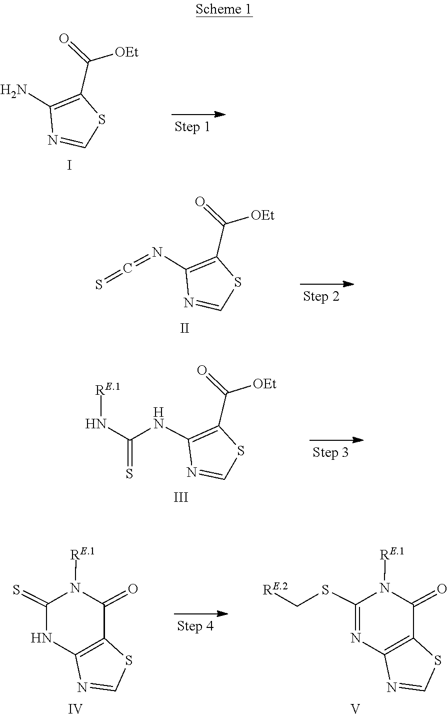

Intermediates II (Step 1, intermediates I→intermediates II) can be prepared by treating intermediates I with a suitable thiocarbonylation reagent, for example 1,1′-thiocarbonyl-bis(pyridin-2(1H)-one) or 1,1-thiocarbonyldiimidazole (TCDI), in a suitable solvent, for example acetonitrile (Scheme 1). Preferred reaction temperatures are between room temperature and 75° C. Formation of the thiourea (Step 2, intermediates II→intermediates III) can be achieved by reacting intermediates II with an aniline either in the presence of a suitable base, such as triethylamine or N,N-diisopropylethylamine, or without base, in a suitable solvent, such as acetonitrile, 2-methyltetrahydrofuran or N,N-dimethylformamide. Preferred reaction temperatures are between room temperature and 70° C. The cyclization (Step 3, intermediates III→intermediates IV) can be achieved by treating intermediates III with a suitable base, such as aqueous sodium hydroxide or lithium hydroxide, in an appropriate solvent, such as methanol, ethanol, tetrahydrofuran or water. Compounds according to the present invention V (step 4, intermediates IV compounds of the invention V) can be prepared by reaction of intermediates IV with a benzyl halide (i.e. chloride or bromide) and a suitable base, such as N,N-diisopropylethylamine or triethylamine, in a suitable solvent, for example N,N-dimethylacetamide, N,N-dimethylformamide, methanol or tetrahydrofuran.

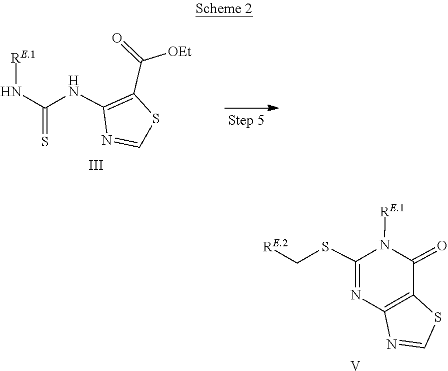

Alternatively, compounds of the present invention V can directly be obtained by reacting intermediates III with a benzyl halide, such as benzyl chloride or benzyl bromide, and a suitable base, such as N,N-diisopropylethylamine or aqueous lithium hydroxide in a suitable solvent, such as N,N-dimethylformamide or tetrahydrofuran, at temperatures between room temperature and 40° C. (Scheme 2).

### EXAMPLES

### Preparation

The compounds according to the invention and their intermediates may be obtained using methods of synthesis which are known to the one skilled in the art and described in the literature of organic synthesis for example using methods described in “Comprehensive Organic Transformations”, 2nd Edition, Richard C. Larock, John Wiley & Sons, 2010, and “March's Advanced Organic Chemistry”, 7th Edition, Michael B. Smith, John Wiley & Sons, 2013. Preferably the compounds are obtained analogously to the methods of preparation explained more fully hereinafter, in particular as described in the experimental section. In some cases, the sequence adopted in carrying out the reaction schemes may be varied. Variants of these reactions that are known to the skilled artisan but are not described in detail herein may also be used. The general processes for preparing the compounds according to the invention will become apparent to the skilled man on studying the schemes that follow. Starting compounds are commercially available or may be prepared by methods that are described in the literature or herein, or may be prepared in an analogous or similar manner. Before the reaction is carried out, any corresponding functional groups in the starting compounds may be protected using conventional protecting groups. These protecting groups may be cleaved again at a suitable stage within the reaction sequence using methods familiar to the skilled man and described in the literature for example in “Protecting Groups”, 3rd Edition, Philip J. Kocienski, Thieme, 2005, and “Protective Groups in Organic Synthesis”, 4th Edition, Peter G. M. Wuts, Theodora W. Greene, John Wiley & Sons, 2006. The terms “ambient temperature” and “room temperature” are used interchangeably and designate a temperature of about 20° C., e.g. between 19 and 24° C.

### Abbreviations

| | |
|---|---|
| ACN | acetonitrile |
| Aq. | aqueous |
| Boc | tert-butyloxycarbonyl |
| ° C. | degree celsius |
| CDI | carbonyl diimidazole |
| CyH/CH | cyclohexane |
| conc. | concentrated |
| DCM | dichloromethane |
| DIPEA | N,N-diisopropylethylamine |
| DMA | N,N-dimethylacetamide |
| DMF | N,N-dimethylformamide |
| DMSO | dimethyl sulfoxide |
| ESI-MS | electrospray ionisation mass spectrometry |
| EtOAc | ethyl acetate |
| EtOH | ethanol |
| ex | example |
| equiv | equivalent |
| FA | formic acid |
| h | hour |
| HATU | 1-[bis(dimethylamino)methylene]-1H-1,2,3-triazolo[4,5- |
| | b]pyridinium 3-oxid hexafluorophosphate |
| HCl | hydrochloric acid |
| HPLC | high performance liquid chromatography |
| Int. | intermediate |
| K₂CO₃ | potassium carbonate |
| KOtBu | potassium tert-butoxide |
| L | liter |
| LiOH*H₂O | lithium hydroxide monohydrate |
| M | molar |
| MeOH | methanol |
| MgSO₄ | magnesium sulphate |
| min | minute |
| mL | milliliter |
| MTBE | tert-butylmethylether |
| NaOEt | sodium ethoxide |
| NH₃ | ammonia |
| PMB | para-methoxybenzyl |
| Prep. | preparative |
| RP | reversed phase |
| Rₜ | retention time |
| sat. | saturated |
| TBTU | 2-(1H-benzotriazole-1-yl)-1,1,3,3-tetramethylaminium |
| | tetrafluoroborate |
| TCDI | 1,1-thiocarbonyldiimidazole |
| TEA | triethylamine |
| TFA | trifluoroacetic acid |
| TFAA | trifluoroacetic anhydride |
| THF | tetrahydrofuran |
| TBS | tert-butyldimethylsilyl |
| TMS | trimethylsilyl |
| TMS-Cl | trimethylsilyl chloride |

### Nomenclature

The names of the compounds described in the experimental section were generated using PerkinElmer ChemDraw® Software (Version 22.2.0.330).

### Preparation of Starting Materials

Unless stated otherwise, the starting materials are commercially available and used without further purification.

The following starting materials are prepared as described in the literature cited:

5-((4-Methoxybenzyl)oxy)pyrazin-2-amine: M. Yamamoto, M. Takadoi, Y. Fukuda, Y. Asahina, Cyclopentylacrylic acid amide derivatives, WO 2009133687, April 2009

### Preparation of Intermediates

### Intermediate 1

### Ethyl 4-isothiocyanatothiazole-5-carboxylate

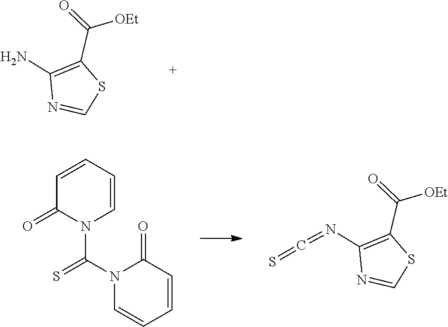

1,1′-Thiocarbonylbis(pyridin-2(1H)-one) (28.3 g, 122 mmol, 1.05 equiv) is added to a solution of ethyl 4-aminothiazole-5-carboxylate (20.0 g, 116 mmol, 1 equiv) in acetonitrile (110 mL), and the reaction mixture is heated to 75° C. After 3 h, the reaction mixture is poured on ice-water, and the mixture is extracted with tert-butylmethylether. The phases are separated, and the organic phase is washed with water and dried over sodium sulfate. All volatiles are removed under reduced pressure, and the residue is purified by flash column chromatography (silica gel, gradient cyclohexane to cyclohexane/EtOAc 70:30) to provide the product.
C₇H₆N₂O₂S₂ (M=214.0 g/mol)
ESI 215 [M+H]⁺
Rₜ (HPLC) 0.96 min (Method A)

### Intermediate 2

### Ethyl 4-(3-(4-hydroxyphenyl)thioureido)thiazole-5-carboxylate

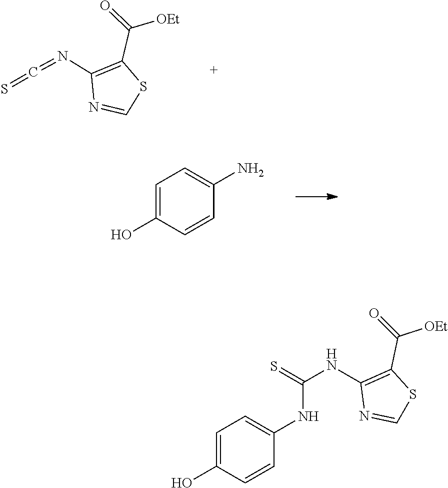 [SMILES: CCOC(=O)c1scnc1N=C=S ; Nc1ccc(O)cc1 ; CCOC(=O)c1scnc1NC(=S)Nc1ccc(O)cc1]

4-Aminophenol (102 mg, 933 μmol, 1 equiv) is added to a solution of ethyl 4-isothiocyanatothiazole-5-carboxylate (intermediate 1, 200 mg, 933 μmol, 1 equiv) in acetonitrile (2 mL), and the mixture is stirred at room temperature. After 18 h, the precipitate is filtered and dried to yield the desired product.
C₁₃H₁₃N₃O₃S₂(M=323.0 g/mol)
ESI 324 [M+H]⁺
Rₜ (HPLC) 0.93 min (Method A)

### Intermediate 3

### 6-(4-Hydroxyphenyl)-5-thioxo-5,6-dihydrothiazolo[4,5-d]pyrimidin-7(4H)-one

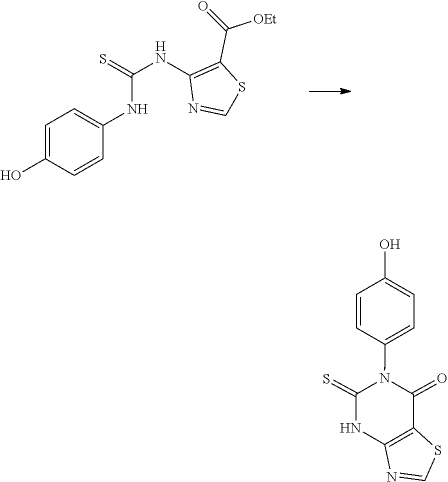 [SMILES: CCOC(=O)c1scnc1NC(=S)Nc1ccc(O)cc1 ; O=c1c2scnc2[nH]c(=S)n1-c1ccc(O)cc1]

A solution of sodium hydroxide (4 M in water, 662 μL, 2.65 mmol, 4.00 equiv) is added to a mixture of ethyl 4-(3-(4-hydroxyphenyl)thioureido)thiazole-5-carboxylate (intermediate 2, 214 mg, 662 μmol, 1 equiv) in methanol (3 mL). After 5 h, the reaction mixture is neutralized with aqueous hydrochloric acid (4 M, 662 μL), and the resulting precipitate is filtered and dried to yield the desired product.
C₁₁H₇N₃O₂S₂ (M=277.0 g/mol)
ESI 278 [M+H]⁺
Rₜ (HPLC) 0.61 min (Method A)

### Intermediate 4

### Ethyl 4-(3-(3,5-difluoro-4-methoxyphenyl)thioureido)thiazole-5-carboxylate

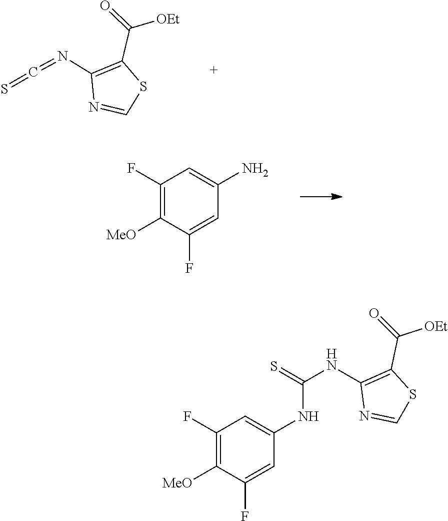 [SMILES: CCOC(=O)c1scnc1N=C=S ; COc1c(F)cc(N)cc1F ; CCOC(=O)c1scnc1NC(=S)Nc1cc(F)c(OC)c(F)c1]

3,5-Difluoro-4-methoxyaniline×H₂SO₄ (2.40 g, 9.33 mmol, 1 equiv) is added to a solution of ethyl 4-isothiocyanatothiazole-5-carboxylate (intermediate 1, 2.00 g, 9.33 mmol, 1 equiv) and triethylamine (1.30 mL, 9.33 mmol, 1 equiv) in acetonitrile (10 mL). After 4 h, the reaction mixture is diluted with diisopropyl ether, and the precipitate is filtered and dried to yield the desired product.
C₁₄H₁₃F₂N₃O₃S₂ (M=373.0 g/mol)
ESI 374 [M+H]⁺
Rₜ (HPLC) 1.20 min (Method B)

### Intermediate 5

### 6-(3,5-Difluoro-4-methoxyphenyl)-5-thioxo-5,6-dihydrothiazolo[4,5-d]pyrimidin-7(4H)-one

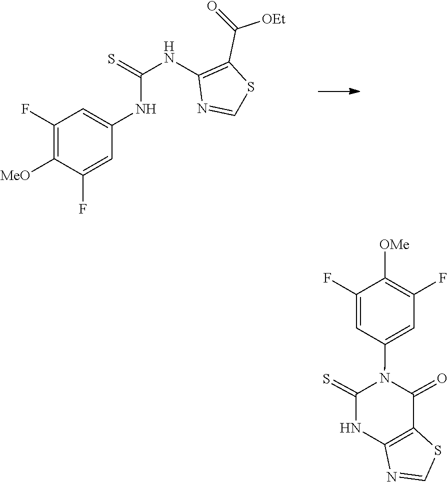 [SMILES: CCOC(=O)c1scnc1NC(=S)Nc1cc(F)c(OC)c(F)c1 ; COc1c(F)cc(-n2c(=S)[nH]c3ncsc3c2=O)cc1F]

A solution of sodium hydroxide (2 M in water, 5.00 mL, 10.0 mmol, 2.00 equiv) is added to a mixture of ethyl 4-(3-(3,5-difluoro-4-methoxyphenyl)thioureido)thiazole-5-carboxylate (intermediate 4, 1.87 g, 5.00 mmol, 1 equiv) in tetrahydrofuran (50 mL). After 4 h, aqueous potassium bisulfate solution is added, and the resulting precipitate is filtered and dried to yield the desired product.
C₁₂H₇F₂N₃O₂S₂(M=327.0 g/mol)
ESI 328 [M+H]⁺
Rₜ (HPLC) 0.91 min (Method A)

### Intermediate 6

6-(3,5-Difluoro-4-hydroxyphenyl)-5-thioxo-5,6-dihydrothiazolo[4,5-d]pyrimidin-7(4H)-one

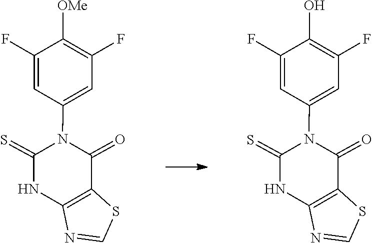 [SMILES: COc1c(F)cc(-n2c(=S)[nH]c3ncsc3c2=O)cc1F ; O=c1c2scnc2[nH]c(=S)n1-c1cc(F)c(O)c(F)c1]

A solution of boron tribromide (1 M in CH₂Cl₂, 19.9 mL, 19.9 mmol, 5.00 equiv) is added to a mixture of 6-(3,5-difluoro-4-methoxyphenyl)-5-thioxo-5,6-dihydrothiazolo[4,5-d]pyrimidin-7(4H)-one (intermediate 5, 1.30 g, 3.97 mmol, 1 equiv) in dichloromethane (20 mL) at −10° C. After complete addition the reaction mixture is heated to 50° C. After 18 h, the reaction mixture is cooled to room temperature and poured on ice-water, and the resulting precipitate is filtered and dried to yield the desired product.
C₁₁H₅F₂N₃O₂S₂(M=313.0 g/mol)
ESI 314 [M+H]⁺
Rₜ (HPLC) 0.74 min (Method B)

### Intermediate 7

### Methyl 4-(3-(5-hydroxypyrimidin-2-yl)thioureido)thiazole-5-carboxylate

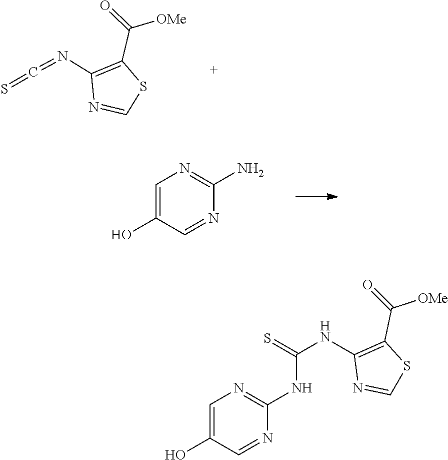 [SMILES: COC(=O)c1scnc1N=C=S ; Nc1ncc(O)cn1 ; COC(=O)c1scnc1NC(=S)Nc1ncc(O)cn1]

Intermediate 7 is prepared using procedures analogous to those described for intermediate 2, using appropriate starting materials.
C₁₀H₉N₅O₃S₂(M=311.0 g/mol)
ESI 312 [M+H]⁺
Rₜ (HPLC) 0.76 min (Method B)

### Intermediate 8

### 6-(5-Hydroxypyrimidin-2-yl)-5-thioxo-5,6-dihydrothiazolo[4,5-d]pyrimidin-7(4H)-one

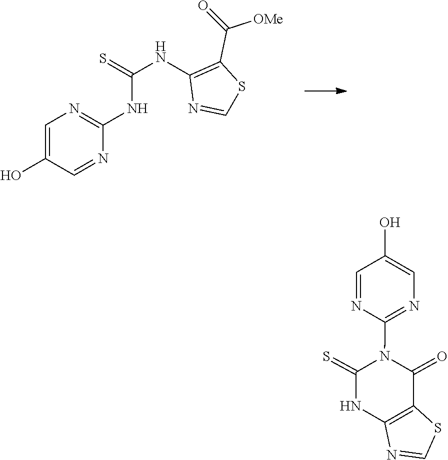 [SMILES: COC(=O)c1scnc1NC(=S)Nc1ncc(O)cn1 ; O=c1c2scnc2[nH]c(=S)n1-c1ncc(O)cn1]

Methyl 4-(3-(5-hydroxypyrimidin-2-yl)thioureido)thiazole-5-carboxylate (intermediate 7, 540 mg, 1.73 mmol, 1 equiv) is added in small portions to a mixture of sodium hydroxide in water (1 M, 3.47 mL, 3.47 mmol, 2.00 equiv) and water (25 mL), and the mixture is stirred at room temperature. After 30 min, aqueous hydrochloric acid (1 M, 650 μL) is added, the precipitate is filtered, washed with water and dried to yield the product.
C₉H₅N₅O₂S₂ (M=279.0 g/mol)
ESI 280 [M+H]⁺
Rₜ (HPLC) 0.58 min (Method F)

### Intermediate 9

### Ethyl 4-(3-(6-methoxypyridin-3-yl)thioureido)thiazole-5-carboxylate

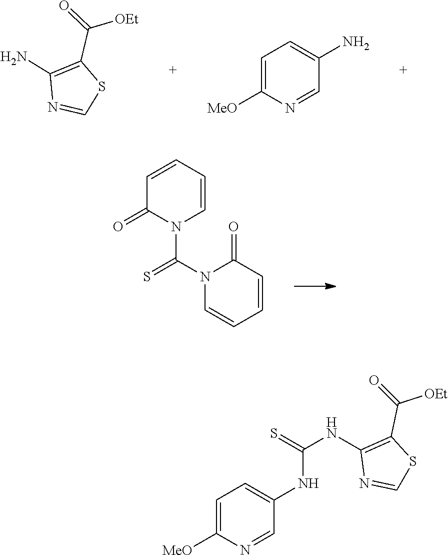 [SMILES: CCOC(=O)c1scnc1N ; COc1ccc(N)cn1 ; O=c1ccccn1C(=S)n1ccccc1=O ; CCOC(=O)c1scnc1NC(=S)Nc1ccc(OC)nc1]

1,1′-Thiocarbonylbis(pyridin-2(1H)-one) (8.09 g, 34.8 mmol, 1.20 equiv) is added to a solution of ethyl 4-aminothiazole-5-carboxylate (5.00 g, 29.0 mmol, 1 equiv) in acetonitrile (100 mL), and the mixture is heated to 70° C. After 18 h, the reaction mixture is cooled to ambient temperature and treated with 6-methoxypyridin-3-amine (5.41 g, 43.6 mmol, 1.50 equiv). After 30 min, water is added, and the resulting precipitate is filtered and dried to yield the product.
C₁₃H₁₄N₄O₃S₂(M=338.1 g/mol)
ESI 339 [M+H]⁺
Rₜ (HPLC) 1.00 min (Method A)

### Intermediate 10

### 6-(6-Methoxypyridin-3-yl)-5-thioxo-5,6-dihydrothiazolo[4,5-d]pyrimidin-7(4H)-one

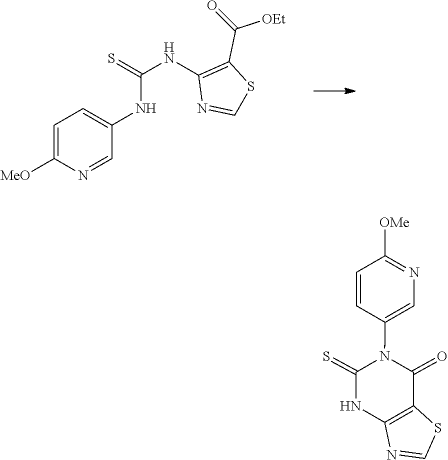 [SMILES: CCOC(=O)c1scnc1NC(=S)Nc1ccc(OC)nc1 ; COc1ccc(-n2c(=S)[nH]c3ncsc3c2=O)cn1]

A solution of aqueous sodium hydroxide (4 M, 3.92 mL, 15.7 mmol, 2.00 equiv) is added to a mixture of ethyl 4-(3-(6-methoxypyridin-3-yl)thioureido)thiazole-5-carboxylate (intermediate 9, 2.65 g, 7.83 mmol, 1 equiv) and ethanol (30 mL). After 30 min, aqueous hydrochloric acid (4 M, 3.92 mL, 15.7 mmol, 2.00 equiv) is added, and the resulting precipitate is filtered and dried to yield the desired product.
C₁₁H₈N₄O₂S₂ (M=292.0 g/mol)
ESI 293 [M+H]⁺
Rₜ (HPLC) 0.70 min (Method A)

### Intermediate 11

### 5-((4-Chloro-2-fluorobenzyl)thio)-6-(6-methoxypyridin-3-yl)thiazolo[4,5-d]pyrimidin-7(6H)-one

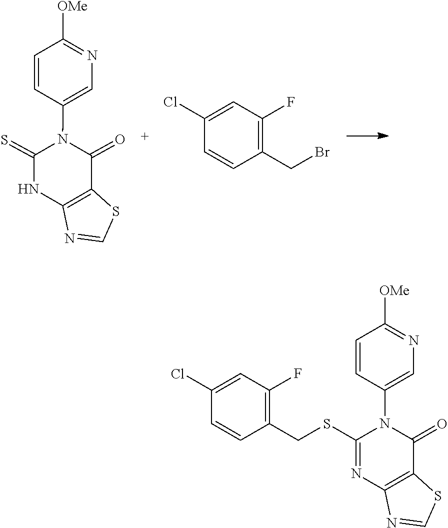 [SMILES: COc1ccc(-n2c(=S)[nH]c3ncsc3c2=O)cn1 ; Fc1cc(Cl)ccc1CBr ; COc1ccc(-n2c(SCc3ccc(Cl)cc3F)nc3ncsc3c2=O)cn1]

4-Chloro-1-(bromomethyl)-2-fluorobenzene (30.6 mg, 171 μmol, 1 equiv) is added to a solution of 6-(6-methoxypyridin-3-yl)-5-thioxo-5,6-dihydrothiazolo[4,5-d]pyrimidin-7(4H)-one (intermediate 10, 50 mg, 171 μmol, 1 equiv) and triethylamine (73.3 μL, 529 μmol, 3.10 equiv) in N,N-dimethylacetamide (2 mL). After 1 h, the reaction mixture is concentrated, and the residue is triturated with water, filtered and dried to yield the desired product.
C₁₈H₁₂ClFN₄O₂S₂ (M=434.0 g/mol)
ESI 435 [M+H]⁺
Rₜ (HPLC) 1.04 min (Method A)

The following intermediates are prepared using procedures analogous to the one described for intermediate 11, using appropriate starting materials. As is appreciated by those skilled in the art, the analogous examples my involve variations in general reaction conditions.

| | | | | HPLC |
|---|---|---|---|---|
| | | | | retention |
| Inter- | Starting | | ESI- | time [min] |
| mediate | material | Structure | MS | (method) |
| 12 | 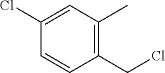 | 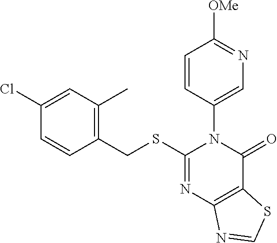 [SMILES: COc1ccc(-n2c(SCc3ccc(Cl)cc3C)nc3ncsc3c2=O)cn1] | 431 [M + H]⁺ | 1.06 (Method A) |
| 13 | 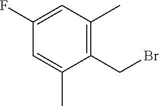 | 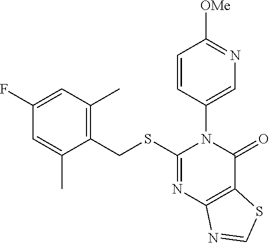 [SMILES: COc1ccc(-n2c(SCc3c(C)cc(F)cc3C)nc3ncsc3c2=O)cn1] | 429 [M + H]⁺ | 1.07 (Method A) |
| 14 | 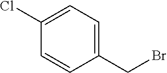 | 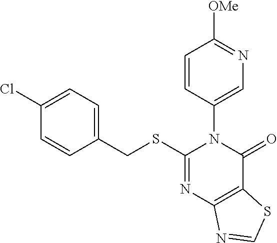 [SMILES: COc1ccc(-n2c(SCc3ccc(Cl)cc3)nc3ncsc3c2=O)cn1] | 417 [M + H]⁺ | 1.01 (Method A) |
| 15 | 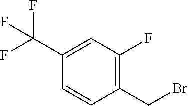 [SMILES: Fc1cc(C(F)(F)F)ccc1CBr] | 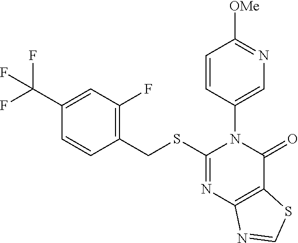 [SMILES: COc1ccc(-n2c(SCc3ccc(C(F)(F)F)cc3F)nc3ncsc3c2=O)cn1] | 469 [M + H]⁺ | 1.05 (Method A) |
| 16 | 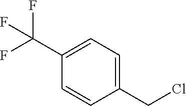 [SMILES: FC(F)(F)c1ccc(CCl)cc1] | 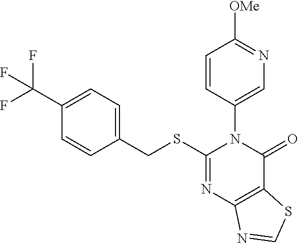 [SMILES: COc1ccc(-n2c(SCc3ccc(C(F)(F)F)cc3)nc3ncsc3c2=O)cn1] | 451 [M + H]⁺ | 1.03 (Method A) |
| 17 | 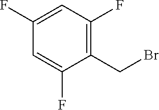 [SMILES: Fc1cc(F)c(CBr)c(F)c1] | 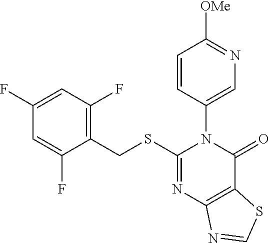 [SMILES: COc1ccc(-n2c(SCc3c(F)cc(F)cc3F)nc3ncsc3c2=O)cn1] | 437 [M + H]⁺ | 1.01 (Method A) |
| 18 | 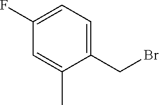 | 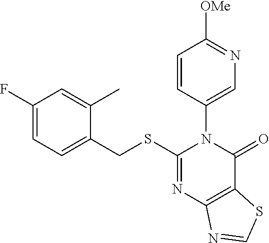 [SMILES: COc1ccc(-n2c(SCc3ccc(F)cc3C)nc3ncsc3c2=O)cn1] | 415 [M + H]⁺ | 1.03 (Method A) |
| 19 | 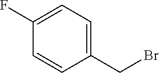 | 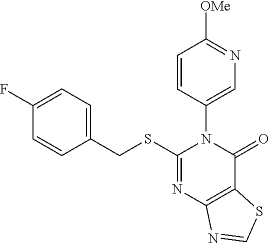 [SMILES: COc1ccc(-n2c(SCc3ccc(F)cc3)nc3ncsc3c2=O)cn1] | 401 [M + H]⁺ | 1.00 (Method A) |

### Intermediate 20

### Ethyl 4-(3-(5-fluoro-6-hydroxypyridin-3-yl)thioureido)thiazole-5-carboxylate

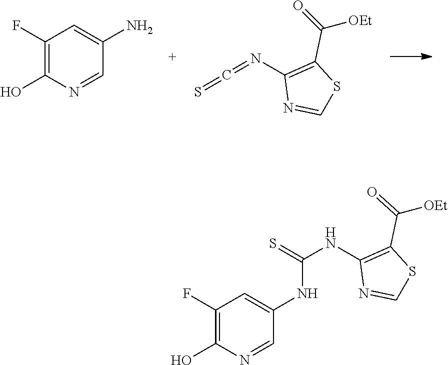 [SMILES: Nc1cnc(O)c(F)c1 ; CCOC(=O)c1scnc1N=C=S ; CCOC(=O)c1scnc1NC(=S)Nc1cnc(O)c(F)c1]

Ethyl 4-isothiocyanatothiazole-5-carboxylate (intermediate 1, 2.00 g. 9.37 mmol, 1 equiv) is added to a solution of 5-amino-3-fluoropyridin-2-ol (1.50 g, 9.37 mmol, 1 equiv) in 2-methyltetrahydrofuran (15 mL). After 18 h, tert-butylmethylether is added, and the precipitate is filtered to yield the product.
C₁₂H₁₁FN₄O₃S₂ (M=342.0 g/mol)
ESI 343 [M+H]⁺
Rₜ (HPLC) 0.89 min (Method B)

### Intermediate 21

### 6-(5-Fluoro-6-hydroxypyridin-3-yl)-5-thioxo-5,6-dihydrothiazolo[4,5-d]pyrimidin-7(4H)-one

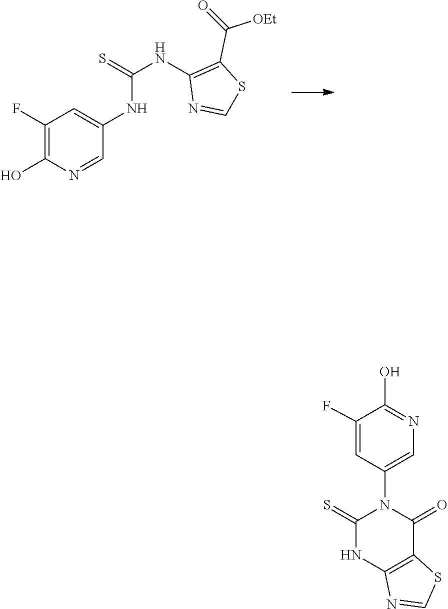 [SMILES: CCOC(=O)c1scnc1NC(=S)Nc1cnc(O)c(F)c1 ; O=c1c2scnc2[nH]c(=S)n1-c1cnc(O)c(F)c1]

Ethyl 4-(3-(5-fluoro-6-hydroxypyridin-3-yl)thioureido)thiazole-5-carboxylate (intermediate 20, 525 mg, 1.53 mmol, 1 equiv) is added in small portions to a solution of sodium hydroxide in water (0.1 M, 12 mL, 1.53 mmol, 1 equiv), and the mixture is stirred at room temperature. After 30 min, the reaction mixture is poured on aqueous potassium bisulfate solution, and the precipitate is filtered and washed with water to yield the product.
C₁₀H₅FN₄O₂S₂ (M=296.0 g/mol)
ESI 297 [M+H]⁺
Rₜ (HPLC) 0.60 min (Method B)

### Intermediate 22

### 2-Methoxy-5-(7-oxo-5-((2,4,6-trifluorobenzyl)thio)thiazolo[4,5-d]pyrimidin-6(7H)-yl)-nicotinonitrile

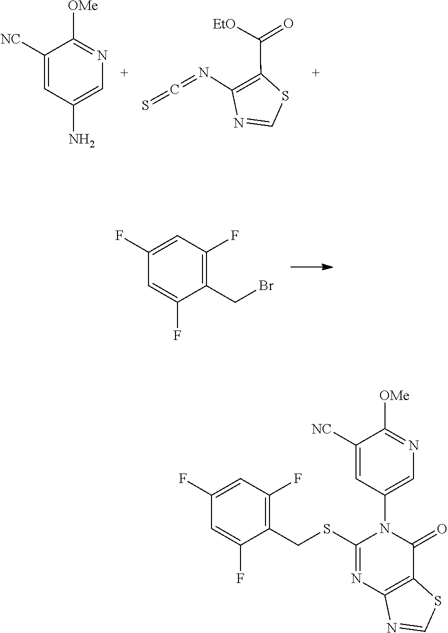

5-Amino-2-methoxynicotinonitrile (15.7 mg, 100 μmol, 1 equiv) is added to a solution of ethyl 4-isothiocyanatothiazole-5-carboxylate (intermediate 1, 21.4 mg, 100 μmol, 1 equiv) and N,N-diisopropylethylamine (30.0 μL, 173 μmol, 2.00 equiv) in N,N-dimethylformamide (2 mL). After 18 h, aqueous lithium hydroxide solution (2 M, 100 μL, 200 μmol, 2.00 equiv) and 2-(bromomethyl)-1,3,5-trifluorobenzene (22.0 mg, 98.0 μmol, 1 equiv) are added. After 18 h, the reaction mixture is filtered, and the filtrate is purified by reversed phase HPLC (Waters Sunfire™-C18, gradient of acetonitrile in water, 0.1% TFA) to yield the desired product.
C₁₉H₁₀F₃N₅O₂S₂(M=461.0 g/mol)
ESI 462 [M+H]⁺
Rₜ (HPLC) 1.10 min (Method A)

### Intermediate 23

### Ethyl 4-(3-(5-chloro-6-hydroxypyridin-3-yl)thioureido)thiazole-5-carboxylate

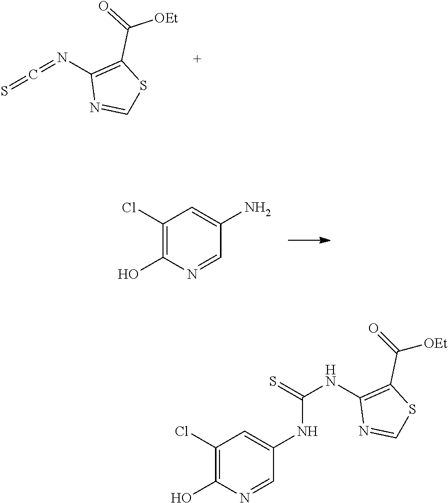 [SMILES: CCOC(=O)c1scnc1N=C=S ; Nc1cnc(O)c(Cl)c1 ; CCOC(=O)c1scnc1NC(=S)Nc1cnc(O)c(Cl)c1]

Intermediate 23 is prepared using procedures analogous to those described for intermediate 20, using appropriate starting materials.
C₁₂H₁₁C₁N₄O₃S₂ (M=358.0 g/mol)
ESI 359 [M+H]⁺
Rₜ (HPLC) 0.50 min (Method C)

### Intermediate 24

### 6-(5-Chloro-6-hydroxypyridin-3-yl)-5-thioxo-5,6-dihydrothiazolo[4,5-d]pyrimidin-7(4H)-one

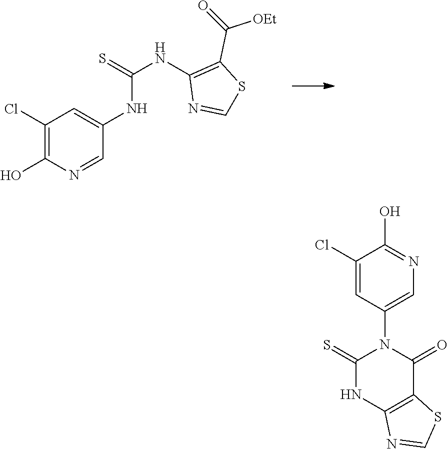 [SMILES: CCOC(=O)c1scnc1NC(=S)Nc1cnc(O)c(Cl)c1 ; O=c1c2scnc2[nH]c(=S)n1-c1cnc(O)c(Cl)c1]

Intermediate 24 is prepared using procedures analogous to those described for intermediate 3, using appropriate starting materials.
C₁₀H₅ClN₄O₂S₂ (M=312.0 g/mol)
ESI 313 [M+H]⁺
Rₜ (HPLC) 0.27 min (Method C)

### Intermediate 25

### Methyl 4-(3-(4,6-difluoropyridin-3-yl)thioureido)thiazole-5-carboxylate

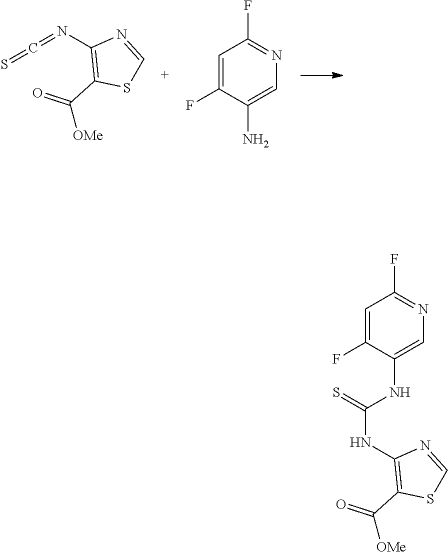 [SMILES: COC(=O)c1scnc1N=C=S ; Nc1cnc(F)cc1F ; COC(=O)c1scnc1NC(=S)Nc1cnc(F)cc1F]

4,6-Difluoropyridin-3-amine (250 mg, 1.86 mmol, 1 equiv) is added to a solution of methyl 4-isothiocyanatothiazole-5-carboxylate (intermediate 53, 373 mg, 1.86 mmol, 1 equiv) in N,N-dimethylacetamide (2 mL). After 18 h, tert-butylmethylether and water are added, and the precipitate is filtered to yield the product.
C₁₁H₈F₂N₄O₂S₂(M=330.0 g/mol)
ESI 331 [M+H]⁺
Rₜ (HPLC) 1.00 min (Method B)

### Intermediate 26

### 6-(4,6-Difluoropyridin-3-yl)-5-((2,4,6-trifluorobenzyl)thio)thiazolo[4,5-d]pyrimidin-7(6H)-one

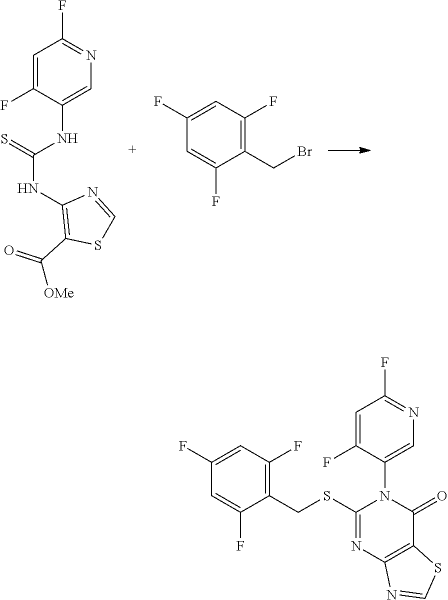 [SMILES: COC(=O)c1scnc1NC(=S)Nc1cnc(F)cc1F ; Fc1cc(F)c(CBr)c(F)c1 ; O=c1c2scnc2nc(SCc2c(F)cc(F)cc2F)n1-c1cnc(F)cc1F]

Methyl 4-(3-(4,6-difluoropyridin-3-yl)thioureido)thiazole-5-carboxylate (intermediate 25, 250 mg, 757 μmol, 1 equiv) is added to a solution of 2-(bromomethyl)-1,3,5-trifluorobenzene (170 mg, 757 μmol, 1 equiv) and N,N-diisopropylethylamine (131 μL, 757 μmol, 1 equiv) in tetrahydrofuran (5 mL). After 2 h, additional 2-(bromomethyl)-1,3,5-trifluorobenzene (170 mg, 757 μmol, 1 equiv) and N,N-diisopropylethylamine (131 μL, 757 μmol, 1 equiv) are added and the mixture is heated to 40° C., After 18 h, the reaction mixture is directly purified by flash column chromatography (silica gel, gradient cyclohexane/EtOAc 90:10 to cyclohexane/EtOAc 50:50) to yield the desired product.
C₁₇H₇F₅N₄O₂S₂ (M=442.0 g/mol)
ESI 443 [M+H]⁺
Rₜ (HPLC) 1.02 min (Method F)

### Intermediate 27

### 6-(4-Fluoro-6-(2-(trimethylsilyl)ethoxy)pyridin-3-yl)-5-((2,4,6-trifluorobenzyl)thio)-thiazolo [4,5-d]pyrimidin-7(6H)-one

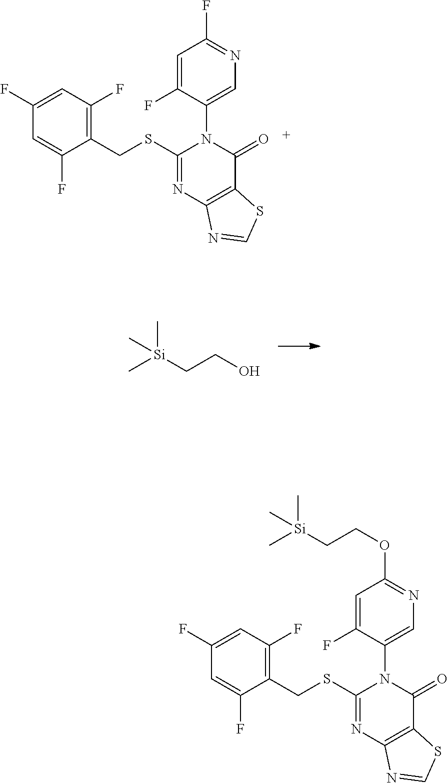

Sodium hydride (55% mineral oil dispersion, 19.7 mg, 452 μmol, 1 equiv) is added to a solution of 2-(trimethylsilyl)ethan-1-ol (66.1 μL, 452 μmol, 1 equiv) in tetrahydrofuran (4 mL). After 10 min, 6-(4,6-difluoropyridin-3-yl)-5-((2,4,6-trifluorobenzyl)thio)thiazolo-[4,5-d]pyrimidin-7(6H)-one (intermediate 26, 200 mg, 452 μmol, 1 equiv) is added. After 18 h, the reaction mixture is filtered, and the residue is purified by preparative reversed phase HPLC (Waters XBridge™-C18, gradient of acetonitrile in water, 0.1% NH₃) and then by flash column chromatography (silica gel, gradient cyclohexane/EtOAc 80:20 to cyclohexane/EtOAc 50:50) to yield the desired product.
C₂₂H₂₀F₄N₄O₂S₂Si (M=540.0 g/mol)
ESI 541 [M+H]⁺
Rₜ (HPLC) 1.27 min (Method F)

### Intermediate 28

### 6-(2-Fluoro-6-methoxypyridin-3-yl)-5-thioxo-5,6-dihydrothiazolo[4,5-d]pyrimidin-7(4H)-one

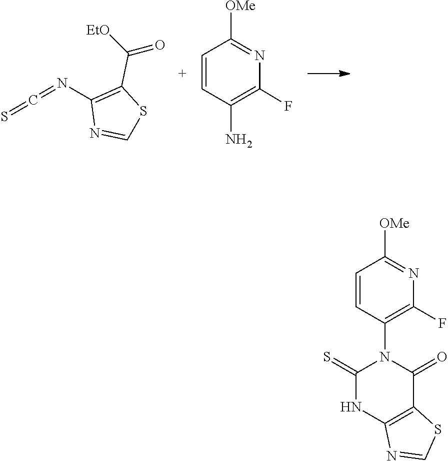

Intermediate 28 is prepared using procedures analogous to those described for example 20, using appropriate starting materials.
C₁₁H₇FN₄O₂S₂ (M=310.0 g/mol)
ESI 311 [M+H]⁺
Rₜ (HPLC) 0.87 min (Method B)

### Intermediate 29

### 6-(2-Fluoro-6-methoxypyridin-3-yl)-5-((2,4,6-trifluorobenzyl)thio)thiazolo[4,5-d]pyrimidin-7(6H)-one

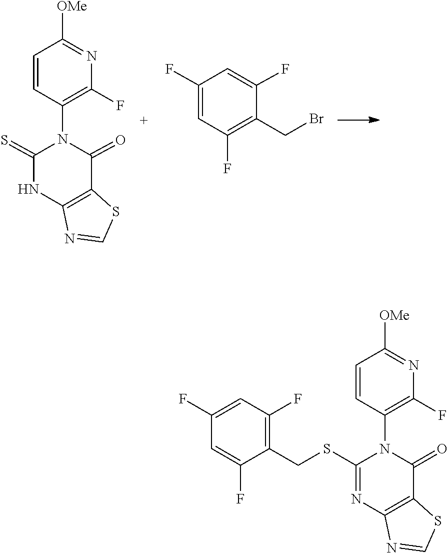 [SMILES: COc1ccc(-n2c(=S)[nH]c3ncsc3c2=O)c(F)n1 ; Fc1cc(F)c(CBr)c(F)c1 ; COc1ccc(-n2c(SCc3c(F)cc(F)cc3F)nc3ncsc3c2=O)c(F)n1]

2-(Bromomethyl)-1,3,5-trifluorobenzene (167 mg, 741 μmol, 1 equiv) is added to a solution of 6-(2-fluoro-6-methoxypyridin-3-yl)-5-thioxo-5,6-dihydrothiazolo[4,5-d]pyrimidin-7(4H)-one (intermediate 28, 230 mg, 741 μmol, 1 equiv) and N,N-diisopropylethylamine (128 μL, 741 μmol, 1 equiv) in N,N-dimethylacetamide (2.3 mL). After 2 h, water is added, and the precipitate is filtered and washed with water to yield the product.
C₁₈H₁₀F₄N₄O₂S₂(M=454.0 g/mol)
ESI 455 [M+H]⁺
Rₜ (HPLC) 1.13 min (Method B)

### Intermediate 30a

### 2,5,6-Trifluoropyridin-3-amine

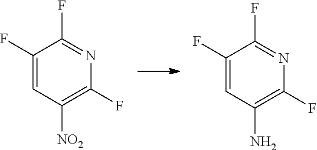 [SMILES: O=[N+]([O-])c1cc(F)c(F)nc1F ; Nc1cc(F)c(F)nc1F]

A mixture of 2,3,6-trifluoro-5-nitropyridine (400 mg, 2.13 mmol, 1 equiv) and Raney-Nickel (30 mg) in methanol (5 mL) is stirred under hydrogen atmosphere (3 bar). After 24 h, the mixture is filtered, and the filtrate is concentrated to yield the product.
C₅H₃F₃N₂(M=148.0 g/mol)
ESI 149 [M+H]⁺
Rₜ (HPLC) 0.67 min (Method A)

### Intermediate 30b

### Ethyl 4-(3-(2,5,6-trifluoropyridin-3-yl)thioureido)thiazole-5-carboxylate

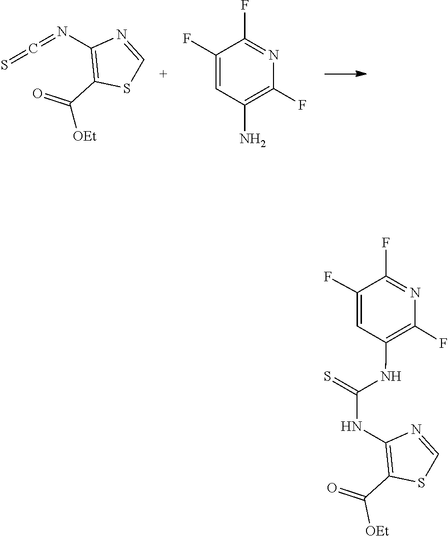 [SMILES: CCOC(=O)c1scnc1N=C=S ; Nc1cc(F)c(F)nc1F ; CCOC(=O)c1scnc1NC(=S)Nc1cc(F)c(F)nc1F]

Intermediate 30b is prepared using procedures analogous to those described for intermediate 2, using appropriate starting materials.
C₁₂H₉F₃N₄O₂S₂(M=362.0 g/mol)
ESI 363 [M+H]⁺
Rₜ (HPLC) 1.11 min (Method A)

### Intermediate 31

### 6-(2,5-Difluoro-6-methoxypyridin-3-yl)-5-((2,4,6-trifluorobenzyl)thio)thiazolo[4,5-d]pyrimidin-7(6H)-one

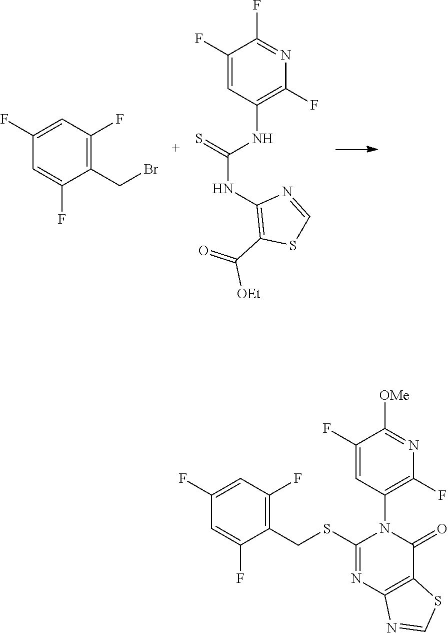 [SMILES: Fc1cc(F)c(CBr)c(F)c1 ; CCOC(=O)c1scnc1NC(=S)Nc1cc(F)c(F)nc1F ; COc1nc(F)c(-n2c(SCc3c(F)cc(F)cc3F)nc3ncsc3c2=O)cc1F]

2-(Bromomethyl)-1,3,5-trifluorobenzene (280 mg, 1.21 mmol, 1 equiv) is added to a solution of ethyl 4-(3-(2,5,6-trifluoropyridin-3-yl)thioureido)thiazole-5-carboxylate (intermediate 30, 568 mg, 1.25 mmol, 1 equiv) and N,N-diisopropylethylamine (420 μL, 2.43 mmol, 1.90 equiv) in N,N-dimethylformamide (2 mL), and the mixture is stirred for 2 h. Water and dichloromethane are added, and the layers are separated. The aqueous layer is extracted with dichloromethane, and the combined organic layers are filtered through a phase transfer filter. The filtrate is concentrated under reduced pressure, and the residue is triturated with a mixture of water and methanol and dried. The residue is treated with a mixture of methanol (2 mL) and aqueous ammonium hydroxide solution (33%, 1 mL). After 48 h, water is added, and the precipitate is filtered and washed with dichloromethane to yield the product.
C₁₈H₉F₅N₄O₂S₂(M=472.0 g/mol)
ESI 473 [M+H]⁺
Rₜ (HPLC) 1.10 min (Method A)

### Intermediate 32

### 4,5-Difluoro-6-methoxynicotinic acid

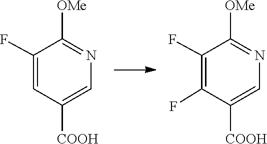 [SMILES: COc1ncc(C(=O)O)cc1F ; COc1ncc(C(=O)O)c(F)c1F]

A solution of n-butyllithium in hexane (2.5 M, 2.22 mL, 5.55 mmol, 1 equiv) is added to a mixture of lithium tetramethylpiperidide (2.56 g, 16.9 mmol, 3.00 equiv) and tetrahydrofuran (5 mL) at −64° C. After 5 min, a mixture of 5-fluoro-6-methoxynicotinic acid (1.00 g. 5.55 mmol, 1 equiv) and tetrahydrofuran (10 mL) is added, and the reaction mixture is allowed to warm to −40° C. After 1.5 h, a mixture of N-fluorobenzenesulfonimide (5.41 g, 16.7 mmol, 3.00 equiv) and tetrahydrofuran (10 mL) is added. After 40 min, dichloromethane and water are added, and the reaction mixture is allowed to warm to room temperature. The layers are separated, and the organic layer is extracted with water. The combined aqueous layers are concentrated, and the residue is purified by reversed phase HPLC (Waters Sunfire™-C18, gradient of acetonitrile in water, 0.1% TFA) to yield the desired product.
C₇H₅F₂NO₃ (M=189.0 g/mol)
ESI 190 [M+H]⁺
Rₜ (HPLC) 0.73 min (Method A)

### Intermediate 33

### Tert-butyl (4,5-difluoro-6-methoxypyridin-3-yl)carbamate

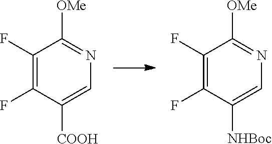 [SMILES: COc1ncc(C(=O)O)c(F)c1F ; COc1ncc(NC(=O)OC(C)(C)C)c(F)c1F]

A mixture of 4,5-difluoro-6-methoxynicotinic acid (intermediate 32, 273 mg, 1.44 mmol, 1 equiv), diphenylphosphoryl azide (401 mg, 1.46 mmol, 1 equiv) and triethylamine (204 μL, 1.45 mmol, 1 equiv) in tert-butanol (6 mL) is heated to 90° C. After 3.5 h, water is added, and the mixture is extracted with dichloromethane. The combined organic layers are filtered through a phase transfer filter and the filtrate is concentrated under reduced pressure.

The residue is purified by flash column chromatography (silica gel, gradient cyclohexane to cyclohexane/EtOAc 90:10) to yield the desired product.
C₁₁H₁₄F₂N₂O₃(M=260.0 g/mol)
ESI 261 [M+H]⁺
Rₜ (HPLC) 0.98 min (Method A)

### Intermediate 34

### 4,5-Difluoro-6-methoxypyridin-3-amine

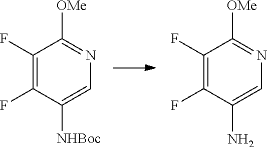 [SMILES: COc1ncc(NC(=O)OC(C)(C)C)c(F)c1F ; COc1ncc(N)c(F)c1F]

A solution of hydrochloric acid in 1,4-dioxane (4 M, 2 mL) is added to tert-butyl (4,5-difluoro-6-methoxypyridin-3-yl)carbamate (intermediate 33, 220 mg, 845 μmol, 1 equiv). After 18 h, the precipitate is filtered and dried to yield the desired product.
C₆H₆F₂N₂₀ (M=160.0 g/mol)
ESI 161 [M+H]⁺
Rₜ (HPLC) 0.64 min (Method A)

### Intermediate 35

### Ethyl 4-(3-(4,5-difluoro-6-methoxypyridin-3-yl)thioureido)thiazole-5-carboxylate

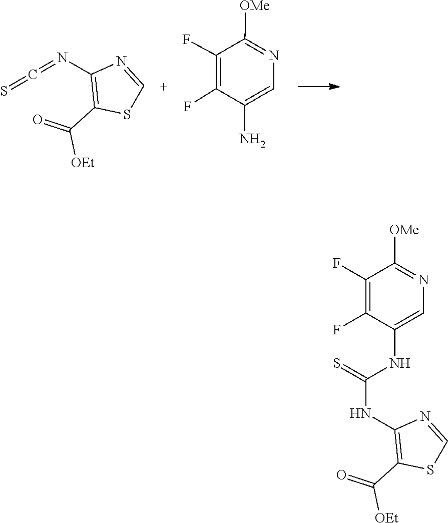 [SMILES: CCOC(=O)c1scnc1N=C=S ; COc1ncc(N)c(F)c1F ; CCOC(=O)c1scnc1NC(=S)Nc1cnc(OC)c(F)c1F]

4,5-Difluoro-6-methoxypyridin-3-amine (intermediate 34, 73.4 mg, 373 μmol, 1 equiv) is added to a solution of ethyl 4-isothiocyanatothiazole-5-carboxylate (intermediate 1, 80.0 mg, 373 μmol, 1 equiv) and triethylamine (130 μL, 933 μmol, 2.50 equiv) in N,N-dimethylformamide (2 mL). After 1.5 h, water is added, and the precipitate is filtered and dried to yield the desired product.
C₁₃H₁₂F₂N₄O₃S₂ (M=374.0 g/mol)
ESI 375 [M+H]⁺
Rₜ (HPLC) 1.07 min (Method A)

### Intermediate 36

### 6-(4,5-Difluoro-6-methoxypyridin-3-yl)-5-((2,4,6-trifluorobenzyl)thio)thiazolo[4,5-d]-pyrimidin-7(6H)-one

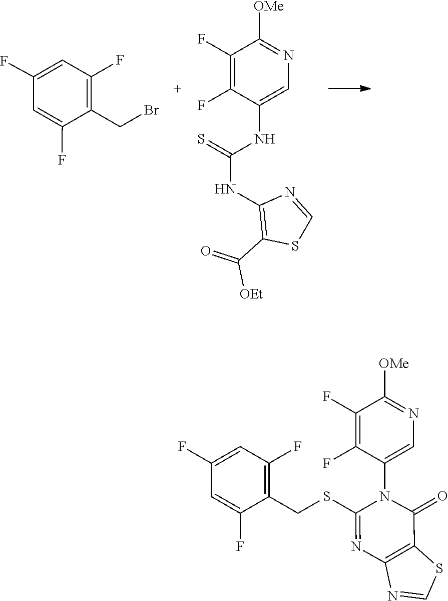 [SMILES: Fc1cc(F)c(CBr)c(F)c1 ; CCOC(=O)c1scnc1NC(=S)Nc1cnc(OC)c(F)c1F ; COc1ncc(-n2c(SCc3c(F)cc(F)cc3F)nc3ncsc3c2=O)c(F)c1F]

2-(Bromomethyl)-1,3,5-trifluorobenzene (29.0 mg, 125 μmol, 1.20 equiv) is added to a solution of ethyl 4-(3-(4,5-difluoro-6-methoxypyridin-3-yl)thioureido)thiazole-5-carboxylate (intermediate 35, 40.0 mg, 107 μmol, 1 equiv) and N,N-diisopropylethylamine (36.0 μL, 208 μmol, 1.90 equiv) in N,N-dimethylformamide (2 mL). After 1.5 h, water is added, and the precipitate is filtered and dried to yield the desired product.
C₁₈H₉F₅N₄O₂S₂(M=472.0 g/mol)
ESI 473 [M+H]⁺
Rₜ (HPLC) 1.10 min (Method A)

### Intermediate 37

### Ethyl 4-(3-(2-methoxypyrimidin-5-yl)thioureido)thiazole-5-carboxylate

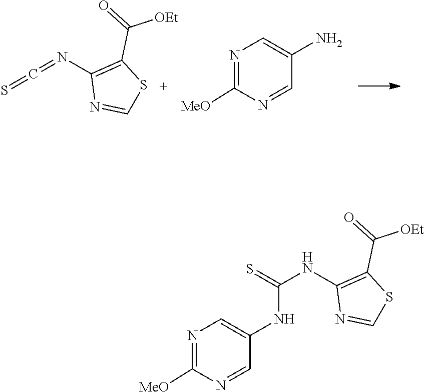 [SMILES: CCOC(=O)c1scnc1N=C=S ; COc1ncc(N)cn1 ; CCOC(=O)c1scnc1NC(=S)Nc1cnc(OC)nc1]

2-Methoxypyrimidin-5-amine (615 mg, 4.67 mmol, 1 equiv) is added to a solution of ethyl 4-isothiocyanatothiazole-5-carboxylate (intermediate 1, 1.00 g, 4.67 mmol, 1 equiv) in acetonitrile (10 mL). After 1 h, the precipitate is filtered and dried to yield the desired product.
C₁₂H₁₃N₅O₃S₂(M=339.0 g/mol)
ESI 340 [M+H]⁺
Rₜ (HPLC) 0.90 min (Method A)

### Intermediate 38

### 6-(2-Methoxypyrimidin-5-yl)-5-thioxo-5,6-dihydrothiazolo[4,5-d]pyrimidin-7(4H)-one

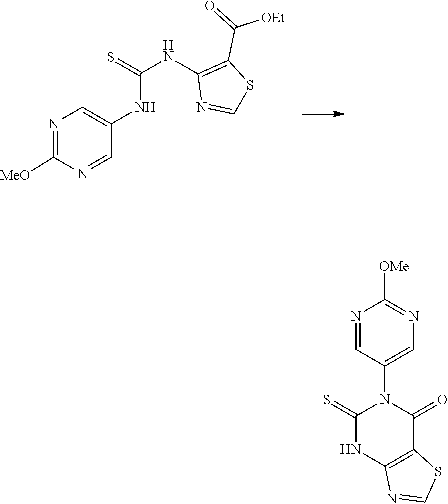 [SMILES: CCOC(=O)c1scnc1NC(=S)Nc1cnc(OC)nc1 ; COc1ncc(-n2c(=S)[nH]c3ncsc3c2=O)cn1]

A solution of sodium hydroxide (4 M in water, 2.11 mL, 8.45 mmol, 2.00 equiv is added to a mixture of ethyl 4-(3-(2-methoxypyrimidin-5-yl)thioureido)thiazole-5-carboxylate (intermediate 37, 1.43 g, 4.23 mmol, 1 equiv) in methanol (15 mL). After 10 min, the precipitate is filtered and dried to yield the desired product.
C₁₀H₇N₅O₂S₂(M=293.0 g/mol)
ESI 294 [M+H]⁺
Rₜ (HPLC) 0.65 min (Method A)

### Intermediate 39

### 5-((4-Chloro-2-fluorobenzyl)thio)-6-(2-methoxypyrimidin-5-yl)thiazolo[4,5-d]pyrimidin-7(6H)-one

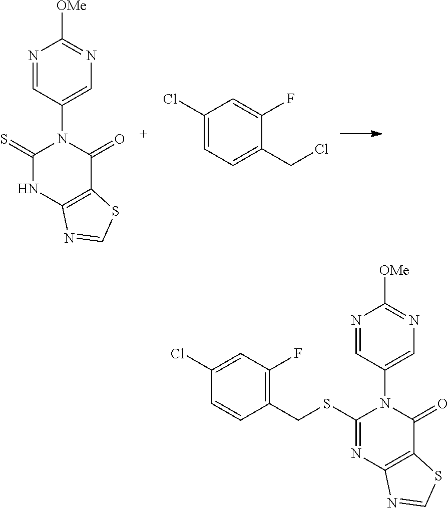 [SMILES: COc1ncc(-n2c(=S)[nH]c3ncsc3c2=O)cn1 ; Fc1cc(Cl)ccc1CCl ; COc1ncc(-n2c(SCc3ccc(Cl)cc3F)nc3ncsc3c2=O)cn1]

4-Chloro-1-(chloromethyl)-2-fluorobenzene (30.5 mg, 170 μmol, 1 equiv) is added to a solution of 6-(2-methoxypyrimidin-5-yl)-5-thioxo-5,6-dihydrothiazolo[4,5-d]pyrimidin-7(4H)-one (intermediate 38, 50 mg, 170 μmol, 1 equiv) and triethylamine (73.0 μL, 527 μmol, 3.10 equiv) in N,N-dimethylacetamide (2 mL). After 1 h, water is added, and the precipitate is filtered and dried to yield the desired product.
C₁₇H₁₁ClFN₅O₂S₂ (M=435.0 g/mol)
ESI 436 [M+H]⁺
Rₜ (HPLC) 0.97 min (Method A)

The following compounds are prepared using procedures analogous to those described for intermediate 39, using appropriate starting materials. As is appreciated by those skilled in the art, the analogous examples my involve variations in general reaction conditions.

| | | | | HPLC retention |
|---|---|---|---|---|
| Inter- | Starting | | | time [min] |
| mediate | material | Structure | ESI-MS | (method) |
| 40 | 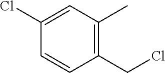 | 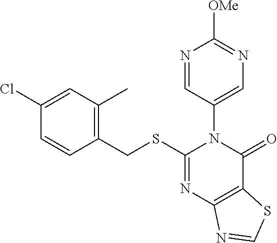 [SMILES: COc1ncc(-n2c(SCc3ccc(Cl)cc3C)nc3ncsc3c2=O)cn1] | 432 [M + H]⁺ | 1.00 (Method A) |
| 41 | 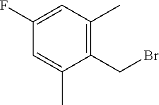 | 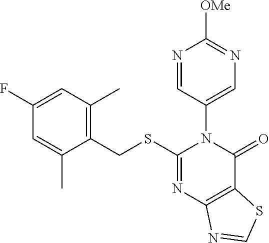 [SMILES: COc1ncc(-n2c(SCc3c(C)cc(F)cc3C)nc3ncsc3c2=O)cn1] | 430 [M + H]⁺ | 0.99 (Method A) |
| 42 | 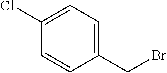 | 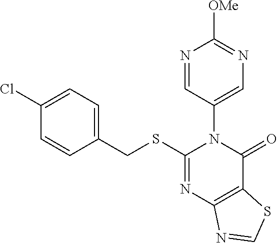 [SMILES: COc1ncc(-n2c(SCc3ccc(Cl)cc3)nc3ncsc3c2=O)cn1] | 418 [M + H]⁺ | 0.96 (Method A) |
| 43 | 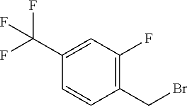 [SMILES: Fc1cc(C(F)(F)F)ccc1CBr] | 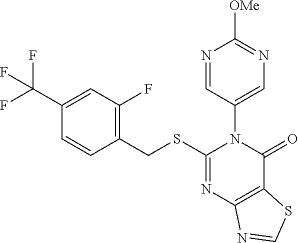 [SMILES: COc1ncc(-n2c(SCc3ccc(C(F)(F)F)cc3F)nc3ncsc3c2=O)cn1] | 470 [M + H]⁺ | 0.99 (Method A) |
| 44 | 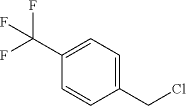 [SMILES: FC(F)(F)c1ccc(CCl)cc1] | 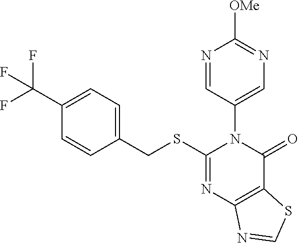 [SMILES: COc1ncc(-n2c(SCc3ccc(C(F)(F)F)cc3)nc3ncsc3c2=O)cn1] | 452 [M + H]⁺ | 0.98 (Method A) |
| 45 | 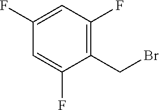 [SMILES: Fc1cc(F)c(CBr)c(F)c1] | 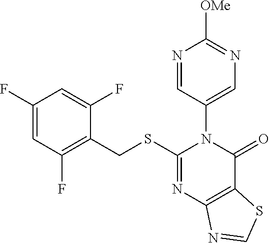 [SMILES: COc1ncc(-n2c(SCc3c(F)cc(F)cc3F)nc3ncsc3c2=O)cn1] | 438 [M + H]⁺ | 0.96 (Method A) |
| 46 | 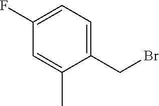 | 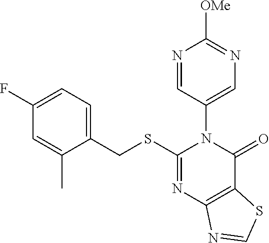 [SMILES: COc1ncc(-n2c(SCc3ccc(F)cc3C)nc3ncsc3c2=O)cn1] | 416 [M + H]⁺ | 0.95 (Method A) |

### Intermediate 47

### Ethyl 4-(3-(6-methoxypyridazin-3-yl)thioureido)thiazole-5-carboxylate

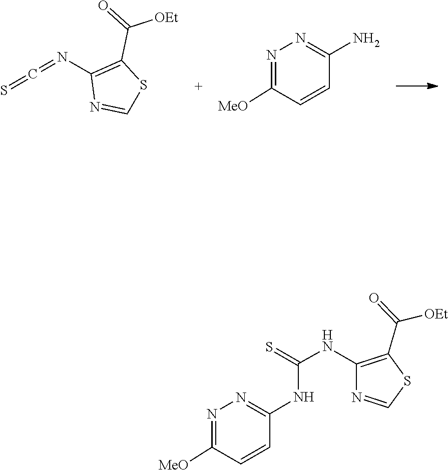 [SMILES: CCOC(=O)c1scnc1N=C=S ; COc1ccc(N)nn1 ; CCOC(=O)c1scnc1NC(=S)Nc1ccc(OC)nn1]

6-Methoxypyridazin-3-amine (291 mg, 2.33 mmol, 1 equiv) is added to a solution of ethyl 4-isothiocyanatothiazole-5-carboxylate (intermediate 1, 500 mg, 2.33 mmol, 1 equiv) in acetonitrile (5 mL). After 1 h, the precipitate is filtered and dried to yield the desired product.
C₁₂H₁₃N₅O₃S₂(M=339.0 g/mol)
ESI 340 [M+H]⁺
Rₜ (HPLC) 0.97 min (Method A)

### Intermediate 48

### 6-(6-Methoxypyridazin-3-yl)-5-thioxo-5,6-dihydrothiazolo[4,5-d]pyrimidin-7(4H)-one

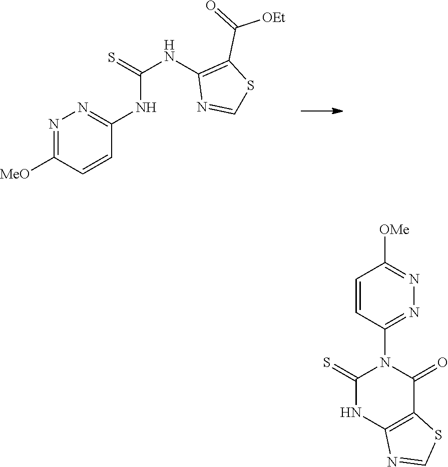 [SMILES: CCOC(=O)c1scnc1NC(=S)Nc1ccc(OC)nn1 ; COc1ccc(-n2c(=S)[nH]c3ncsc3c2=O)nn1]

Aqueous sodium hydroxide (4 M, 803 μL, 3.21 mmol, 2.00 equiv) is added to a mixture of ethyl 4-(3-(6-methoxypyridazin-3-yl)thioureido)thiazole-5-carboxylate (intermediate 47, 545 mg, 1.61 mmol, 1 equiv) in methanol (4 mL). After 18 h, aqueous hydrochloric acid (4 M, 803 μL, 2.00 equiv) and water are added, and the precipitate is filtered and dried to yield the desired product.
C₁₀H₇N₅O₂S₂(M=293.0 g/mol)
ESI 294 [M+H]⁺
Rₜ (HPLC) 0.63 min (Method A)

### Intermediate 49

### 6-(6-Hydroxypyridazin-3-yl)-5-thioxo-5,6-dihydrothiazolo[4,5-d]pyrimidin-7(4H)-one

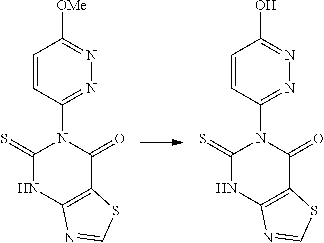 [SMILES: COc1ccc(-n2c(=S)[nH]c3ncsc3c2=O)nn1 ; O=c1c2scnc2[nH]c(=S)n1-c1ccc(O)nn1]

Trimethylsilyl chloride (604 μL, 4.77 mmol, 4.50 equiv) is added to mixture of 6-(6-methoxypyridazin-3-yl)-5-thioxo-5,6-dihydrothiazolo[4,5-d]pyrimidin-7(4H)-one (intermediate 48, 386 mg, 1.05 mmol, 1 equiv) and potassium iodide (157 mg, 947 μmol, 0.900 equiv) in acetonitrile (10 mL). After 18 h, water is added and the precipitate is filtered and dried to yield the desired product.
C₉H₅N₅O₂S₂ (M=279.0 g/mol)
ESI 280 [M+H]⁺
Rₜ (HPLC) 0.47 min (Method A)

### Intermediate 50

### Ethyl 4-(3-(5-methoxypyrazine-2-yl)thioureido)thiazole-5-carboxylate

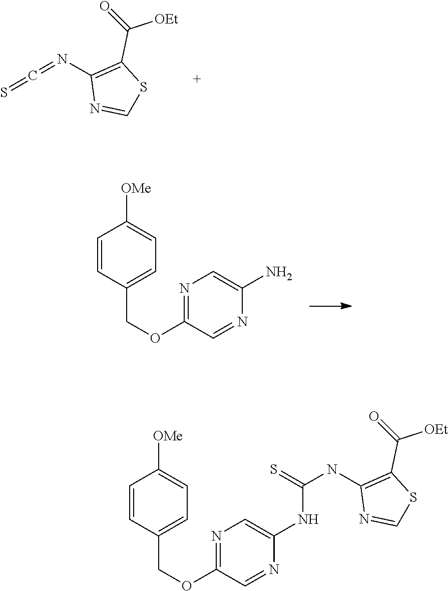

Intermediate 50 is prepared using procedures analogous to those described for intermediate 2, using appropriate starting materials.
C₁₉H₁₉N₅O₄S₂(M=445.1 g/mol)
ESI 446 [M+H]⁺
Rₜ (HPLC) 1.13 min (Method A)

### Intermediate 51

### 6-(5-((4-Methoxybenzyl)oxy)pyrazin-2-yl)-5-thioxo-5,6-dihydrothiazolo[4,5-d]pyrimidin-7(4H)-one

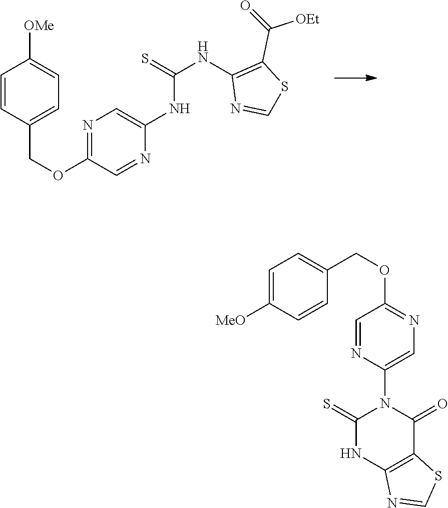 [SMILES: CCOC(=O)c1scnc1NC(=S)Nc1cnc(OCc2ccc(OC)cc2)cn1 ; COc1ccc(COc2cnc(-n3c(=S)[nH]c4ncsc4c3=O)cn2)cc1]

Intermediate 51 is prepared using procedures analogous to those described for intermediate 3, using appropriate starting materials.
C₁₇H₁₃N₅O₃S₂(M=399.0 g/mol)
ESI 400 [M+H]⁺
Rₜ (HPLC) 0.92 min (Method A)

### Intermediate 52

### 6-(5-((4-Methoxybenzyl)oxy)pyrazin-2-yl)-5-((2,4,6-trifluorobenzyl)thio)thiazolo[4,5-d]-pyrimidin-7(6H)-one

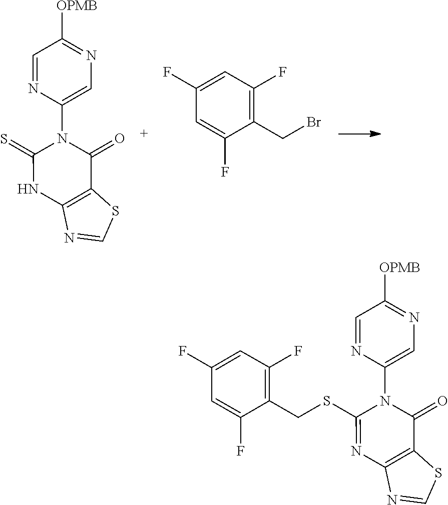

Intermediate 52 is prepared using procedures analogous to those described for intermediate 11, using appropriate starting materials.
C₂₄H₁₆F₃N₅O₃S₂ (M=543.1 g/mol)
ESI 544 [M+H]⁺
Rₜ (HPLC) 1.09 min (Method A)

### Intermediate 53

### Methyl 4-isothiocyanatothiazole-5-carboxylate

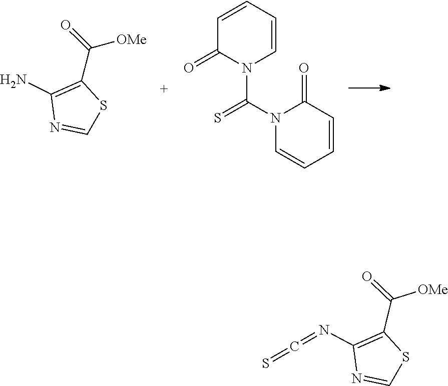 [SMILES: COC(=O)c1scnc1N ; O=c1ccccn1C(=S)n1ccccc1=O ; COC(=O)c1scnc1N=C=S]

Intermediate 53 is prepared in analogy to intermediate 1, replacing ethyl 4-aminothiazole-5-carboxylate with methyl 4-aminothiazole-5-carboxylate as starting material.
C₆H₄N₂O₂S₂ (M=200.0 g/mol)
ESI 201 [M+H]⁺
Rₜ (HPLC) 0.86 min (Method A)

### Preparation of Final Compounds

### Example 1

### 5-((3-Fluorobenzyl)thio)-6-(4-hydroxyphenyl)thiazolo[4,5-d]pyrimidin-7(6H)-one

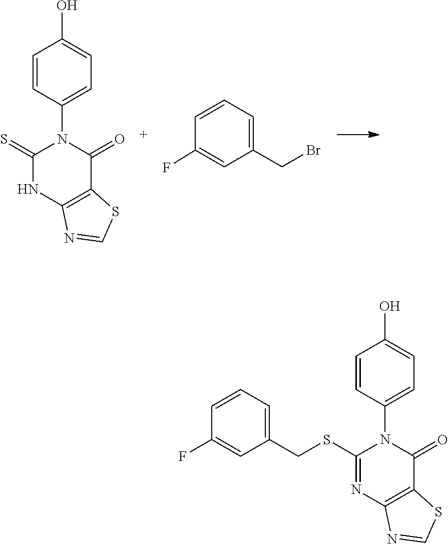 [SMILES: O=c1c2scnc2[nH]c(=S)n1-c1ccc(O)cc1 ; Fc1cccc(CBr)c1 ; O=c1c2scnc2nc(SCc2cccc(F)c2)n1-c1ccc(O)cc1]

1-(Bromomethyl)-3-fluorobenzene (34.1 mg, 180 μmol, 1 equiv) is added to a solution of 6-(4-hydroxyphenyl)-5-thioxo-5,6-dihydrothiazolo[4,5-d]pyrimidin-7(4H)-one (intermediate 3, 50.0 mg, 180 μmol, 1 equiv) and triethylamine (77.2 μL, 557 μmol, 3.10 equiv) in N,N-dimethylacetamide (1 mL). After 18 h, the reaction mixture is filtered, and the filtrate is purified by reversed phase HPLC (Waters Sunfire™-C18, gradient of acetonitrile in water, 0.1% TFA) to yield the desired product.
C₁₈H₁₂FN₃O₂S₂ (M=385.0 g/mol)
ESI 386 [M+H]⁺
Rₜ (HPLC) 0.91 min (Method A)

### Example 2

### 5-((4-Chloro-2-fluorobenzyl)thio)-6-(3,5-difluoro-4-hydroxyphenyl)thiazolo[4,5-d]-pyrimidin-7(6H)-one

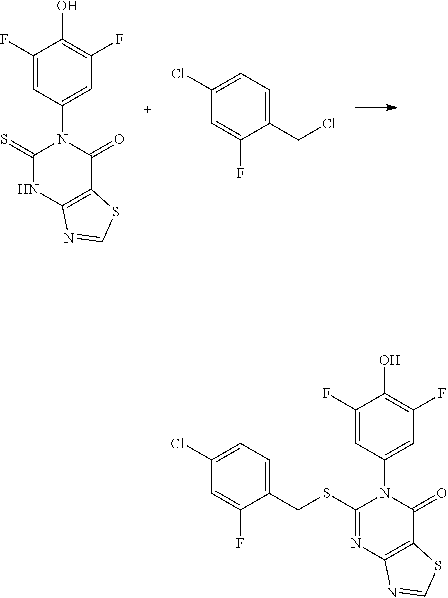 [SMILES: O=c1c2scnc2[nH]c(=S)n1-c1cc(F)c(O)c(F)c1 ; Fc1cc(Cl)ccc1CCl ; O=c1c2scnc2nc(SCc2ccc(Cl)cc2F)n1-c1cc(F)c(O)c(F)c1]

4-Chloro-1-(chloromethyl)-2-fluorobenzene (14.3 mg, 80.0 μmol, 1 equiv) is added to a solution of 6-(3,5-difluoro-4-hydroxyphenyl)-5-thioxo-5,6-dihydrothiazolo[4,5-d]pyri-midin-7(4H)-one (intermediate 6, 25.1 mg, 80.0 μmol, 1 equiv) and N,N-diisopropylethylamine (25.0 μL, 145 μmol, 1.80 equiv) in N,N-dimethylformamide (2 mL). After 18 h, the reaction mixture is filtered and the filtrate is purified by reversed phase HPLC (Waters Sunfire™-C18, gradient of acetonitrile in water, 0.1% TFA) to yield the desired product.
C₁₈H₉C₁F₃N₃O₂S₂(M=455.0 g/mol)
ESI 456 [M+H]⁺
Rₜ (HPLC) 0.92 min (Method G)

The following compounds are prepared using procedures analogous to those described for example 2, general procedure, using appropriate starting materials. As is appreciated by those skilled in the art, the analogous examples my involve variations in general reaction conditions.

| | | | | HPLC retention |
|---|---|---|---|---|
| | Starting | | | time [min] |
| Example | material | Structure | ESI-MS | (method) |
| 3 | 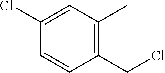 | 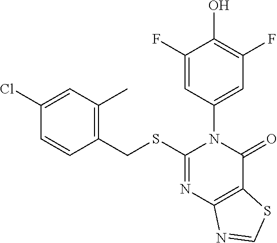 [SMILES: Cc1cc(Cl)ccc1CSc1nc2ncsc2c(=O)n1-c1cc(F)c(O)c(F)c1] | 452 [M + H]⁺ | 0.95 (Method G) |
| 4 | 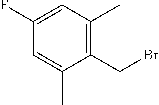 | 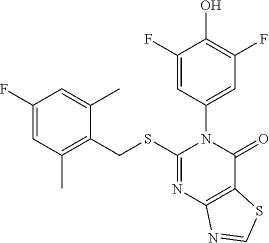 [SMILES: Cc1cc(F)cc(C)c1CSc1nc2ncsc2c(=O)n1-c1cc(F)c(O)c(F)c1] | 450 [M + H]⁺ | 0.93 (Method G) |
| 5 | 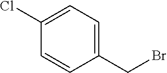 | 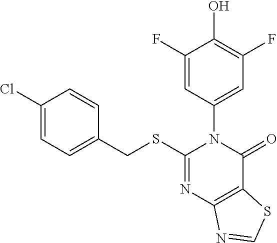 [SMILES: O=c1c2scnc2nc(SCc2ccc(Cl)cc2)n1-c1cc(F)c(O)c(F)c1] | 438 [M + H]⁺ | 0.90 (Method G) |
| 6 | 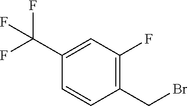 [SMILES: Fc1cc(C(F)(F)F)ccc1CBr] | 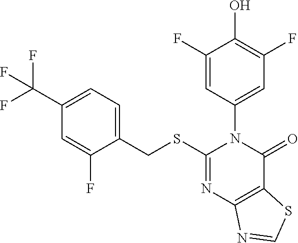 [SMILES: O=c1c2scnc2nc(SCc2ccc(C(F)(F)F)cc2F)n1-c1cc(F)c(O)c(F)c1] | 490 [M + H]⁺ | 0.94 (Method G) |
| 7 | 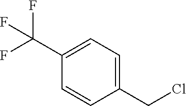 [SMILES: FC(F)(F)c1ccc(CCl)cc1] | 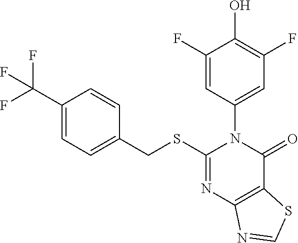 [SMILES: O=c1c2scnc2nc(SCc2ccc(C(F)(F)F)cc2)n1-c1cc(F)c(O)c(F)c1] | 472 [M + H]⁺ | 0.93 (Method G) |
| 8 | 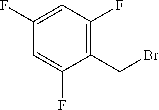 [SMILES: Fc1cc(F)c(CBr)c(F)c1] | 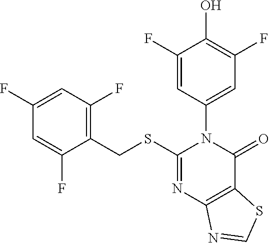 [SMILES: O=c1c2scnc2nc(SCc2c(F)cc(F)cc2F)n1-c1cc(F)c(O)c(F)c1] | 458 [M + H]⁺ | 0.94 (Method A) |
| 9 | 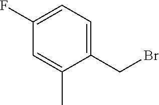 | 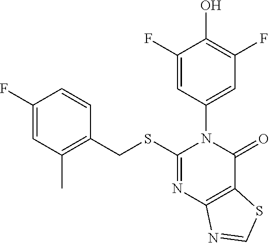 [SMILES: Cc1cc(F)ccc1CSc1nc2ncsc2c(=O)n1-c1cc(F)c(O)c(F)c1] | 436 [M + H]⁺ | 0.88 (Method G) |

### Example 10

### 6-(2,5-Difluoro-4-hydroxyphenyl)-5-((2,4,6-trifluorobenzyl)thio)thiazolo[4,5-d]pyrimidin-7(6H)-one

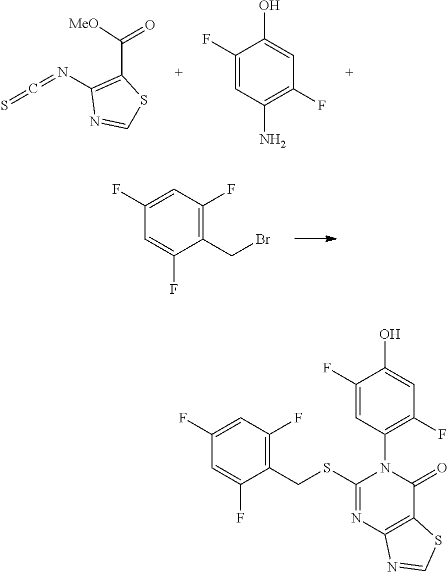 [SMILES: COC(=O)c1scnc1N=C=S ; Nc1cc(F)c(O)cc1F ; Fc1cc(F)c(CBr)c(F)c1 ; O=c1c2scnc2nc(SCc2c(F)cc(F)cc2F)n1-c1cc(F)c(O)cc1F]

A mixture of 4-amino-2,5-difluorophenol (72.6 mg, 500 μmol, 1 equiv), methyl 4-isothiocyanatothiazole-5-carboxylate (100 mg, 500 μmol, 1 equiv) and N,N-diisopropylethylamine (150 μL, 867 μmol, 1.70 equiv) in N,N-dimethylformamide (5 mL) is heated to 55° C. After 3 h, the reaction mixture is cooled to ambient temperature, and aqueous lithium hydroxide solution (2 M, 500 μL, 1.00 mmol, 2.00 equiv) is added. After 2 h, 2-(bromomethyl)-1,3,5-trifluorobenzene (110 mg, 500 μmol, 1 equiv) is added. After 30 min, the reaction mixture is purified by reversed phase HPLC (Waters Sunfire™_C₁₈, gradient of acetonitrile in water, 0.1% TFA) to yield the desired product.
C₁₈H₈F₅N₃O₂S₂(M=457.0 g/mol)
ESI 458 [M+H]⁺
Rₜ (HPLC) 1.05 min (Method A)

The following compounds are prepared using procedures analogous to those described for example 10, using appropriate starting materials. As is appreciated by those skilled in the art, the analogous examples my involve variations in general reaction conditions.

| | | | | HPLC retention |
|---|---|---|---|---|
| | Starting | | | time [min] |
| Example | material | Structure | ESI-MS | (method) |
| 11 | 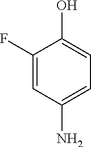 | 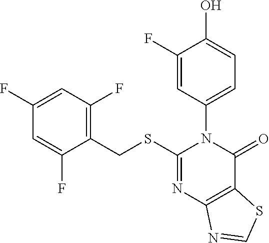 [SMILES: O=c1c2scnc2nc(SCc2c(F)cc(F)cc2F)n1-c1ccc(O)c(F)c1] | 440 [M + H]⁺ | 1.02 (Method A) |
| 12 | 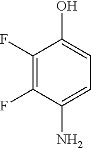 | 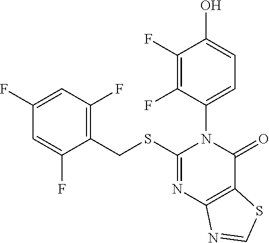 [SMILES: O=c1c2scnc2nc(SCc2c(F)cc(F)cc2F)n1-c1ccc(O)c(F)c1F] | 458 [M + H]⁺ | 1.05 (Method A) |
| 13 | 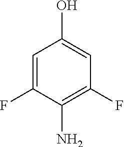 | 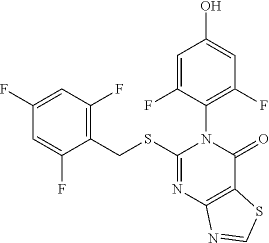 [SMILES: O=c1c2scnc2nc(SCc2c(F)cc(F)cc2F)n1-c1c(F)cc(O)cc1F] | 458 [M + H]⁺ | 0.93 (Method E) |
| 14 | 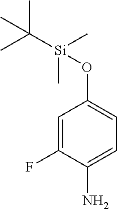 | 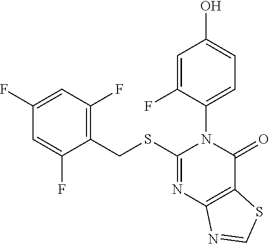 [SMILES: O=c1c2scnc2nc(SCc2c(F)cc(F)cc2F)n1-c1ccc(O)cc1F] | 440 [M + H]⁺ | 1.04 (Method A) |
| 15 | 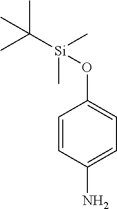 | 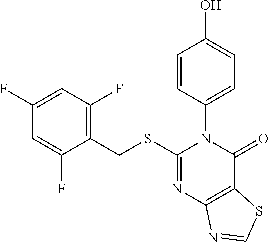 [SMILES: O=c1c2scnc2nc(SCc2c(F)cc(F)cc2F)n1-c1ccc(O)cc1] | 422 [M + H]⁺ | 1.01 (Method A) |
| 16 | 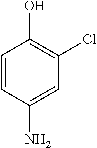 | 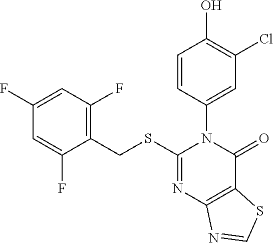 [SMILES: O=c1c2scnc2nc(SCc2c(F)cc(F)cc2F)n1-c1ccc(O)c(Cl)c1] | 456 [M + H]⁺ | 1.05 (Method A) |
| 17 | 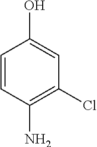 | 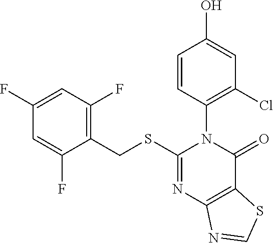 [SMILES: O=c1c2scnc2nc(SCc2c(F)cc(F)cc2F)n1-c1ccc(O)cc1Cl] | 456 [M + H]⁺ | 0.90 (Method E) |
| 18 | 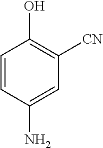 | 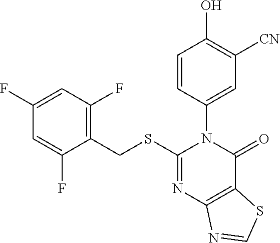 [SMILES: N#Cc1cc(-n2c(SCc3c(F)cc(F)cc3F)nc3ncsc3c2=O)ccc1O] | 447 [M + H]⁺ | 0.83 (Method E) |
| 19 | 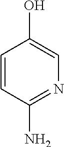 | 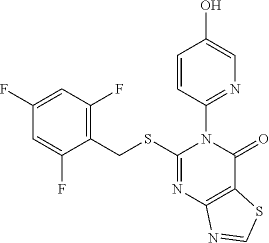 [SMILES: O=c1c2scnc2nc(SCc2c(F)cc(F)cc2F)n1-c1ccc(O)cn1] | 423 [M + H]⁺ | 0.37 (Method D) |
| 20 | 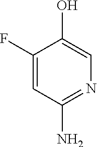 | 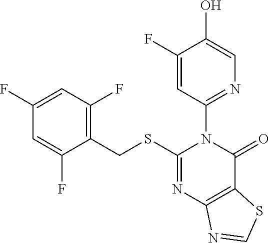 [SMILES: O=c1c2scnc2nc(SCc2c(F)cc(F)cc2F)n1-c1cc(F)c(O)cn1] | 441 [M + H]⁺ | 0.98 (Method A) |

### Example 21

### 6-(5-Hydroxypyrimidin-2-yl)-5-((2,4,6-trifluorobenzyl)thio)thiazolo[4,5-d]pyrimidin-7(6H)-one

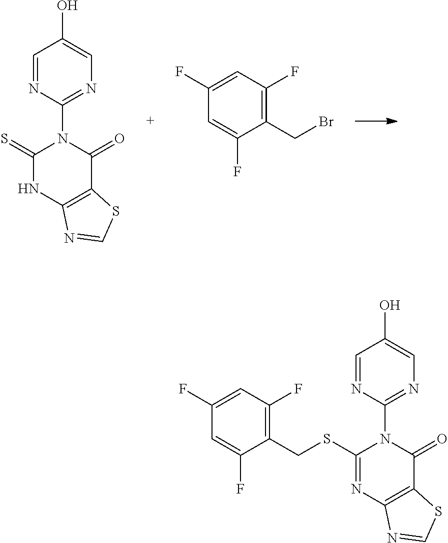 [SMILES: O=c1c2scnc2[nH]c(=S)n1-c1ncc(O)cn1 ; Fc1cc(F)c(CBr)c(F)c1 ; O=c1c2scnc2nc(SCc2c(F)cc(F)cc2F)n1-c1ncc(O)cn1]

Example 21 is prepared using procedures analogous to those described for example 1, using appropriate starting materials.
C₁₆H₈F₃N₅O₂S₂(M=423.0 g/mol)
ESI 424 [M+H]⁺
Rₜ (HPLC) 0.64 min (Method F)

### Example 22

### 5-((4-Chloro-2-fluorobenzyl)thio)-6-(6-hydroxypyridin-3-yl)thiazolo[4,5-d]pyrimidin-7(6H)-one

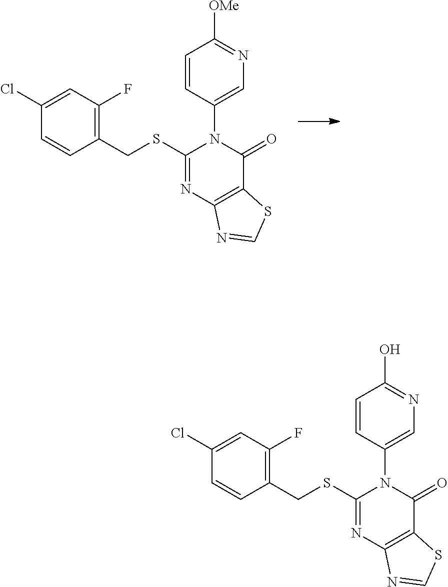 [SMILES: COc1ccc(-n2c(SCc3ccc(Cl)cc3F)nc3ncsc3c2=O)cn1 ; O=c1c2scnc2nc(SCc2ccc(Cl)cc2F)n1-c1ccc(O)nc1]

Trimethylsilyl chloride (79.3 μL, 624 μmol, 4.50 equiv) is added to mixture of 5-((4-chloro-2-fluorobenzyl)thio)-6-(6-hydroxypyridin-3-yl)thiazolo[4,5-d]pyrimidin-7(6H)-one (intermediate 11, 60.0 mg, 138 μmol, 1 equiv) and potassium iodide (20.6 mg, 124 μmol, 0.9 equiv) in acetonitrile (2 mL). After 18 h, the mixture is diluted with N,N-dimethylacetamide, and the mixture is purified by reversed phase HPLC (Waters Sunfire™_C₁₈, gradient of acetonitrile in water, 0.1% TFA) to yield the desired product.
C₁₇H₁₀ClFN₄O₂S₂ (M=420.0 g/mol)
ESI 421 [M+H]⁺
Rₜ (HPLC) 0.72 min (Method E)

The following compounds are prepared using procedures analogous to those described for example 22, using appropriate starting materials. As is appreciated by those skilled in the art, the analogous examples my involve variations in general reaction conditions.

| | | | HPLC retention time |
|---|---|---|---|
| Example | Structure | ESI-MS | [min] (method) |
| 23 | 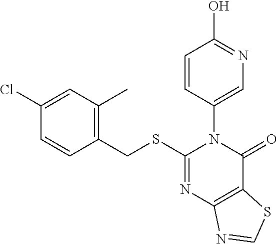 [SMILES: Cc1cc(Cl)ccc1CSc1nc2ncsc2c(=O)n1-c1ccc(O)nc1] | 417 [M + H]⁺ | 0.73 (Method E) |
| 24 | 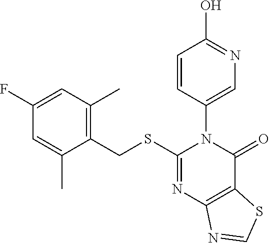 [SMILES: Cc1cc(F)cc(C)c1CSc1nc2ncsc2c(=O)n1-c1ccc(O)nc1] | 415 [M + H]⁺ | 0.85 (Method A) |
| 25 | 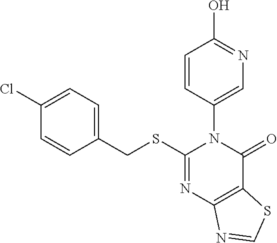 [SMILES: O=c1c2scnc2nc(SCc2ccc(Cl)cc2)n1-c1ccc(O)nc1] | 403 [M + H]⁺ | 0.70 (Method E) |
| 26 | 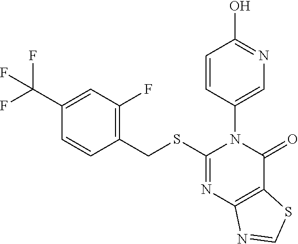 [SMILES: O=c1c2scnc2nc(SCc2ccc(C(F)(F)F)cc2F)n1-c1ccc(O)nc1] | 455 [M + H]⁺ | 0.76 (Method E) |
| 27 | 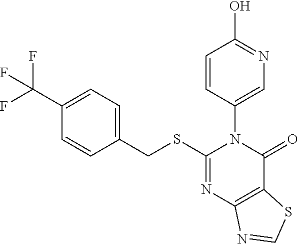 [SMILES: O=c1c2scnc2nc(SCc2ccc(C(F)(F)F)cc2)n1-c1ccc(O)nc1] | 437 [M + H]⁺ | 0.75 (Method E) |
| 28 | 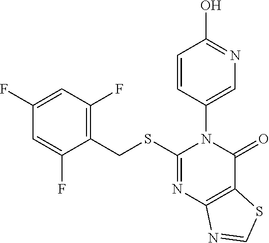 [SMILES: O=c1c2scnc2nc(SCc2c(F)cc(F)cc2F)n1-c1ccc(O)nc1] | 423 [M + H]⁺ | 0.79 (Method E) |
| 29 | 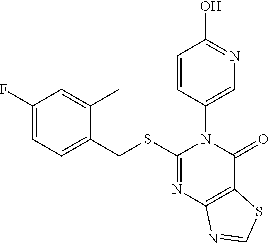 [SMILES: Cc1cc(F)ccc1CSc1nc2ncsc2c(=O)n1-c1ccc(O)nc1] | 401 [M + H]⁺ | 0.82 (Method E) |
| 30 | 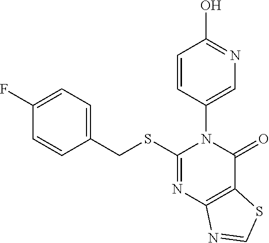 [SMILES: O=c1c2scnc2nc(SCc2ccc(F)cc2)n1-c1ccc(O)nc1] | 387 [M + H]⁺ | 0.64 (Method E) |

### Example 31 (General Procedure A)

### 6-(5-Fluoro-6-hydroxypyridin-3-yl)-5-((2,4,6-trifluorobenzyl)thio)thiazolo[4,5-d]-pyrimidin-7(6H)-one

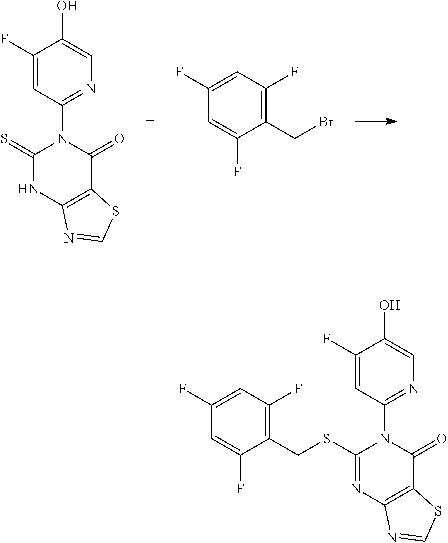 [SMILES: O=c1c2scnc2[nH]c(=S)n1-c1cc(F)c(O)cn1 ; Fc1cc(F)c(CBr)c(F)c1 ; O=c1c2scnc2nc(SCc2c(F)cc(F)cc2F)n1-c1cc(F)c(O)cn1]

Triethylamine (72.0 μL, 522 μmol, 3.10 equiv) is added to a solution of 6-(5-fluoro-6-hydroxypyridin-3-yl)-5-thioxo-5,6-dihydrothiazolo[4,5-d]pyrimidin-7(4H)-one (intermediate 21, 50 mg, 169 μmol, 1 equiv) and 2-(bromomethyl)-1,3,5-trifluorobenzene (38.0 mg, 170 μmol, 1 equiv) in N,N-dimethylacetamide (2 mL). After 40 min, the reaction mixture is filtered, and the filtrate is purified by reversed phase HPLC (Waters XBridge™-C18, gradient of acetonitrile in water, 0.1% TFA) to yield the desired product.
C₁₇H₈F₄N₄O₂S₂(M=440.0 g/mol)
ESI 441 [M+H]⁺
Rₜ (HPLC) 0.48 min (Method C)

### Example 32 (General Procedure B)

### 6-(5-Fluoro-6-hydroxypyridin-3-yl)-5-((4-(trifluoromethyl)benzyl)thio)thiazolo[4,5-d]pyrimidin-7(6H)-one

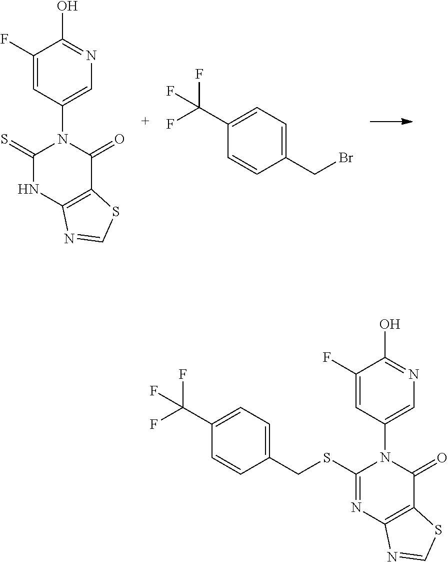 [SMILES: O=c1c2scnc2[nH]c(=S)n1-c1cnc(O)c(F)c1 ; FC(F)(F)c1ccc(CBr)cc1 ; O=c1c2scnc2nc(SCc2ccc(C(F)(F)F)cc2)n1-c1cnc(O)c(F)c1]

Triethylamine (423 μL, 3.04 mmol, 3.00 equiv) is added to a solution of 6-(5-fluoro-6-hydroxypyridin-3-yl)-5-thioxo-5,6-dihydrothiazolo[4,5-d]pyrimidin-7(4H)-one (intermediate 21, 300 mg, 1.01 mmol, 1 equiv) and 1-(bromomethyl)-4-(trifluoromethyl)benzene (242 mg, 1.01 mmol, 1 equiv) in methanol (20 mL). After 1 h, the reaction mixture is poured on water and acidified with hydrochloric acid. The resulting precipitate is filtered and purified by flash column chromatography (silica gel, gradient dichloromethane to dichloromethane/methanol 90:10) to yield the desired product.
C₁₈H₁₀F₄N₄O₂S₂(M=454.0 g/mol)
ESI 455 [M+H]⁺
Rₜ (HPLC) 0.54 min (Method C)

The following compounds are prepared using procedures analogous to those described for example 31 or 32, general procedure A or B, using appropriate starting materials. As is appreciated by those skilled in the art, the analogous examples my involve variations in general reaction conditions.

| | | | | | HPLC |
|---|---|---|---|---|---|
| | | | | | retention |
| Ex- | | | Pro- | | time [min] |
| ample | Starting material | Structure | cedure | ESI-MS | (method) |
| 33 | 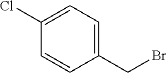 | 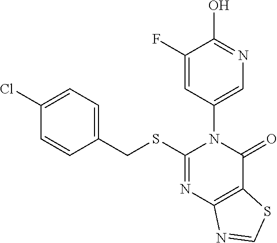 [SMILES: O=c1c2scnc2nc(SCc2ccc(Cl)cc2)n1-c1cnc(O)c(F)c1] | A | 421 [M + H]⁺ | 0.94 (Method B) |
| 34 | 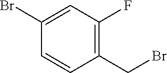 | 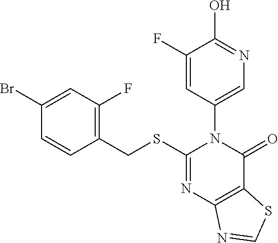 [SMILES: O=c1c2scnc2nc(SCc2ccc(Br)cc2F)n1-c1cnc(O)c(F)c1] | A | 483/485 [M + H]⁺ | 0.53 (Method C) |
| 35 | 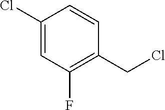 | 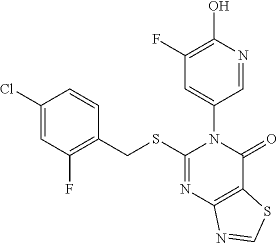 [SMILES: O=c1c2scnc2nc(SCc2ccc(Cl)cc2F)n1-c1cnc(O)c(F)c1] | A | 439 [M + H]⁺ | 0.755 (Method G) |
| 36 | 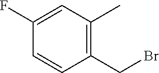 | 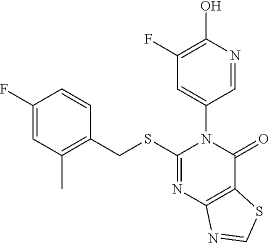 [SMILES: Cc1cc(F)ccc1CSc1nc2ncsc2c(=O)n1-c1cnc(O)c(F)c1] | A | 419 [M + H]⁺ | 0.72 (Method G) |
| 37 | 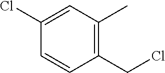 | 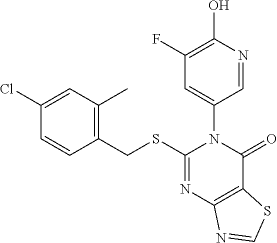 [SMILES: Cc1cc(Cl)ccc1CSc1nc2ncsc2c(=O)n1-c1cnc(O)c(F)c1] | A | 435 [M + H]⁺ | 0.79 (Method G) |
| 38 | 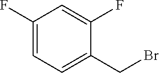 | 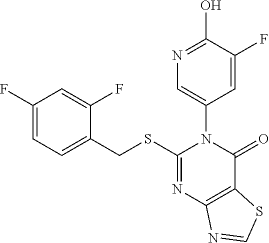 [SMILES: O=c1c2scnc2nc(SCc2ccc(F)cc2F)n1-c1cnc(O)c(F)c1] | A | 423 [M + H]⁺ | 0.48 (Method C) |
| 39 | 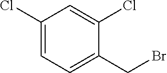 | 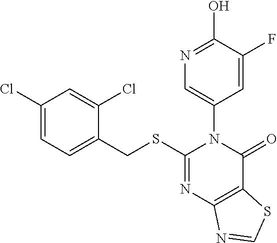 [SMILES: O=c1c2scnc2nc(SCc2ccc(Cl)cc2Cl)n1-c1cnc(O)c(F)c1] | A | 455 [M + H]⁺ | 0.55 (Method C) |
| 40 | 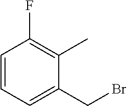 | 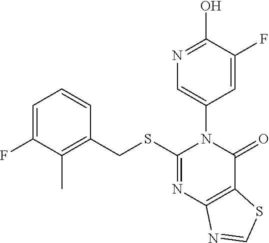 [SMILES: Cc1c(F)cccc1CSc1nc2ncsc2c(=O)n1-c1cnc(O)c(F)c1] | A | 419 [M + H]⁺ | 0.73 (Method G) |
| 41 | 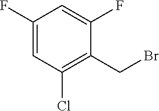 [SMILES: Fc1cc(F)c(CBr)c(Cl)c1] | 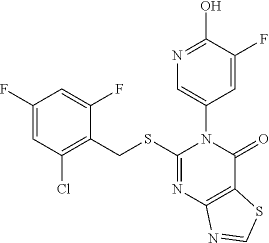 [SMILES: O=c1c2scnc2nc(SCc2c(F)cc(F)cc2Cl)n1-c1cnc(O)c(F)c1] | A | 457 [M + H]⁺ | 0.51 (Method C) |
| 42 | 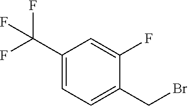 [SMILES: Fc1cc(C(F)(F)F)ccc1CBr] | 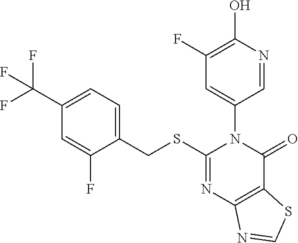 [SMILES: O=c1c2scnc2nc(SCc2ccc(C(F)(F)F)cc2F)n1-c1cnc(O)c(F)c1] | B | 473 [M + H]⁺ | 0.54 (Method C) |
| 43 | 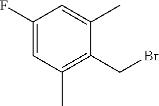 | 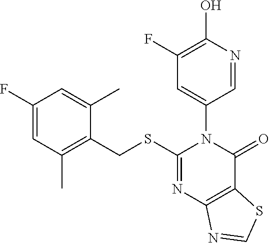 [SMILES: Cc1cc(F)cc(C)c1CSc1nc2ncsc2c(=O)n1-c1cnc(O)c(F)c1] | A | 433 [M + H]⁺ | 0.769 (Method G) |
| 44 | 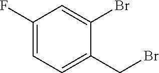 | 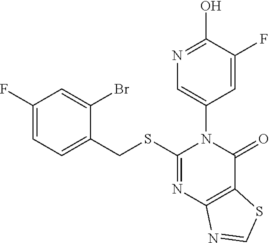 [SMILES: O=c1c2scnc2nc(SCc2ccc(F)cc2Br)n1-c1cnc(O)c(F)c1] | A | 485 [M + H]⁺ | 0.52 (Method C) |
| 45 | 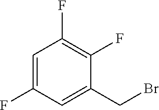 [SMILES: Fc1cc(F)c(F)c(CBr)c1] | 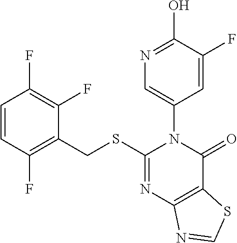 [SMILES: O=c1c2scnc2nc(SCc2c(F)ccc(F)c2F)n1-c1cnc(O)c(F)c1] | B | 441 [M + H]⁺ | 0.50 (Method C) |
| 46 | 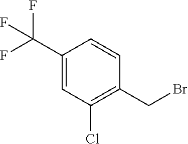 [SMILES: FC(F)(F)c1ccc(CBr)c(Cl)c1] | 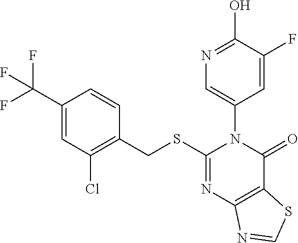 [SMILES: O=c1c2scnc2nc(SCc2ccc(C(F)(F)F)cc2Cl)n1-c1cnc(O)c(F)c1] | A | 489 [M + H]⁺ | 0.57 (Method C) |

### Example 47

### 2-Hydroxy-5-(7-oxo-5-((2,4,6-trifluorobenzyl)thio)thiazolo[4,5-d]pyrimidin-6(7H)-yl)-nicotinonitrile

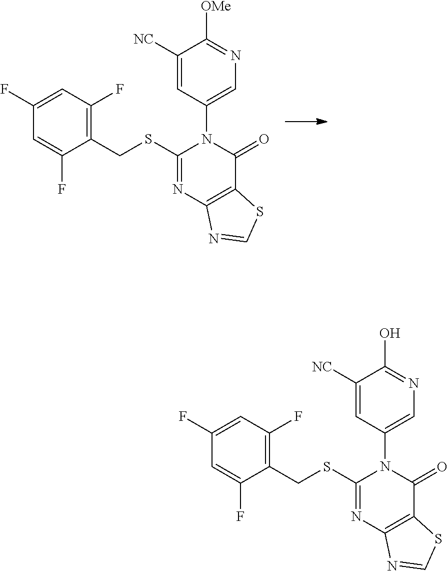 [SMILES: COc1ncc(-n2c(SCc3c(F)cc(F)cc3F)nc3ncsc3c2=O)cc1C#N ; N#Cc1cc(-n2c(SCc3c(F)cc(F)cc3F)nc3ncsc3c2=O)cnc1O]

Trimethylsilyl chloride (20.0 μL, 158 μmol, 4.30 equiv) is added to mixture of 2-methoxy-5-(7-oxo-5-((2,4,6-trifluorobenzyl)thio)thiazolo[4,5-d]pyrimidin-6(7H)-yl)nicotinonitrile (intermediate 22, 17.0 mg, 37.0 μmol, 1 equiv) and sodium iodide (4.97 mg, 33.0 μmol, 0.9 equiv) in acetonitrile (0.5 mL). After 5 h, trimethylsilyl chloride (20.0 μL, 158 μmol, 4.30 equiv) is added, and the mixture is heated to 45° C. After 18 h, the mixture is diluted with N,N-dimethylacetamide, and the mixture is purified by reversed phase HPLC (Waters Sunfire™-C18, gradient of acetonitrile in water, 0.1% TFA), to yield the desired product.
C₁₈H₈F₃N₅O₂S₂(M=447.0 g/mol)
ESI 448 [M+H]⁺
Rₜ (HPLC) 0.70 min (Method E)

### Example 48

### 6-(5-Chloro-6-hydroxypyridin-3-yl)-5-((2,4,6-trifluorobenzyl)thio)thiazolo[4,5-d]pyri-midin-7(6H)-one

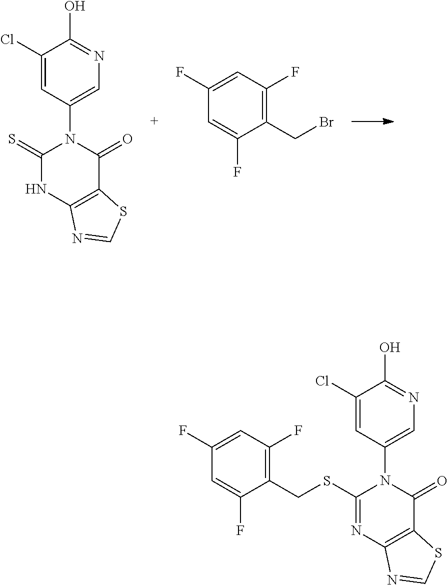 [SMILES: O=c1c2scnc2[nH]c(=S)n1-c1cnc(O)c(Cl)c1 ; Fc1cc(F)c(CBr)c(F)c1 ; O=c1c2scnc2nc(SCc2c(F)cc(F)cc2F)n1-c1cnc(O)c(Cl)c1]

Example 48 is prepared using procedures analogous to those described for example 31, using appropriate starting materials.
C₁₇H₈ClF₃N₄O₂S₂(M=456.0 g/mol)
ESI 457 [M+H]⁺
Rₜ (HPLC) 0.53 min (Method C)

### Example 49

### 6-(4-Fluoro-6-hydroxypyridin-3-yl)-5-((2,4,6-trifluorobenzyl)thio)thiazolo[4,5-d]pyrimidin-7(6H)-one

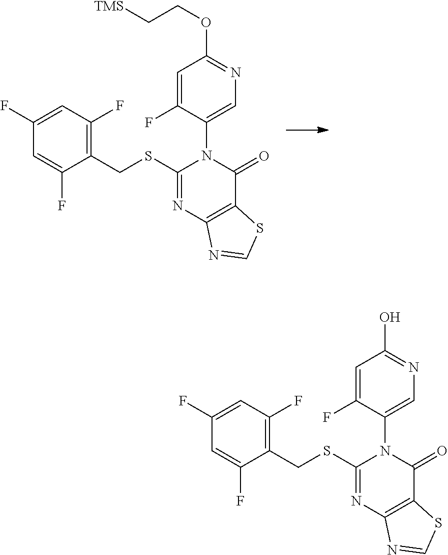 [SMILES: C[Si](C)(C)CCOc1cc(F)c(-n2c(SCc3c(F)cc(F)cc3F)nc3ncsc3c2=O)cn1 ; O=c1c2scnc2nc(SCc2c(F)cc(F)cc2F)n1-c1cnc(O)cc1F]

Trifluoroacetic acid (214 μL, 2.78 mmol, 50.0 equiv) is added to mixture of 6-(4-fluoro-6-(2-(trimethylsilyl)ethoxy)pyridin-3-yl)-5-((2,4,6-trifluorobenzyl)thio)thiazolo[4,5-d]-pyrimidin-7(6H)-one (intermediate 27, 30.0 mg, 55.0 μmol, 1 equiv) in dichloromethane (1.5 mL). After 1 h, the mixture is concentrated under reduced pressure, and the residue is triturated with water, filtered and dried to yield the desired product.
C₁₇H₈F₄N₄O₂S₂(M=440.0 g/mol
ESI 441 [M+H]⁺
Rₜ (HPLC) 0.71 min (Method F)

### Example 50

### 6-(2-Fluoro-6-hydroxypyridin-3-yl)-5-((2,4,6-trifluorobenzyl)thio)thiazolo[4,5-d]-pyrimidin-7(6H)-one

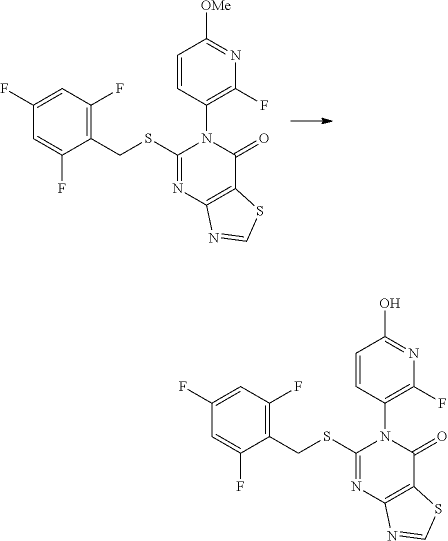 [SMILES: COc1ccc(-n2c(SCc3c(F)cc(F)cc3F)nc3ncsc3c2=O)c(F)n1 ; O=c1c2scnc2nc(SCc2c(F)cc(F)cc2F)n1-c1ccc(O)nc1F]

A solution of boron tribromide in dichloromethane (1 M, 22.5 mL, 22.5 mnmol, 37.9 equiv) is added to neat 6-(2-fluoro-6-methoxypyridin-3-yl)-5-((2,4,6-trifluorobenzyl)thio)thiazolo-[4,5-d]pyrimidin-7(6H)-one (intermediate 29, 270 mg, 594 μmol, 1 equiv), and the reaction mixture is heated to 50° C. After 18 h, the mixture is diluted with dichloromethane and poured on ice-water. The layers are separated, and the organic layer is washed with water and concentrated under reduced pressure. The residue is purified by flash column chromatography (silica gel, gradient cyclohexane/EtOAc 90:10 to cyclohexane/EtOAc 20:80) to yield the desired product.
C₁₇H₈F₄N₄O₂S₂(M=440.0 g/mol)
ESI 441 [M+H]⁺
Rₜ (HPLC) 1.00 min (Method B)

### Example 51

### 6-(2,5-Difluoro-6-hydroxypyridin-3-yl)-5-((2,4,6-trifluorobenzyl)thio)thiazolo[4,5-d]-pyrimidin-7(6H)-one

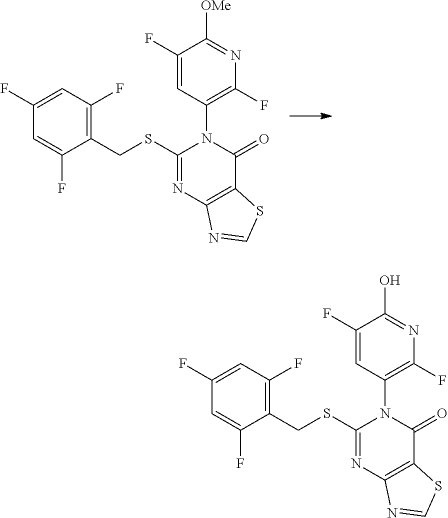 [SMILES: COc1nc(F)c(-n2c(SCc3c(F)cc(F)cc3F)nc3ncsc3c2=O)cc1F ; O=c1c2scnc2nc(SCc2c(F)cc(F)cc2F)n1-c1cc(F)c(O)nc1F]

Example 51 is prepared using procedures analogous to those described for example 50, using appropriate starting materials.
C₁₇H₇F₅N₄O₂S₂(M=458.0 g/mol)
ESI 459 [M+H]⁺
Rₜ (HPLC) 0.95 min (Method A)

### Example 52

### 6-(4,5-Difluoro-6-hydroxypyridin-3-yl)-5-((2,4,6-trifluorobenzyl)thio)thiazolo[4,5-d]-pyrimidin-7(6H)-one

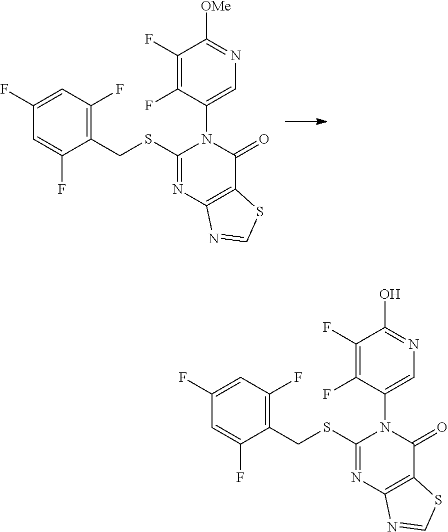 [SMILES: COc1ncc(-n2c(SCc3c(F)cc(F)cc3F)nc3ncsc3c2=O)c(F)c1F ; O=c1c2scnc2nc(SCc2c(F)cc(F)cc2F)n1-c1cnc(O)c(F)c1F]

Example 52 is prepared using procedures analogous to those described for example 47, using appropriate starting materials.
C₁₇H₇F₅N₄O₂S₂(M=458.0 g/mol)
ESI 459 [M+H]⁺
Rₜ (HPLC) 0.89 min (Method A)

### Example 53

### 5-((4-Chloro-2-fluorobenzyl)thio)-6-(2-hydroxypyrimidin-5-yl)thiazolo[4,5-d]pyrimidin-7(6H)-one

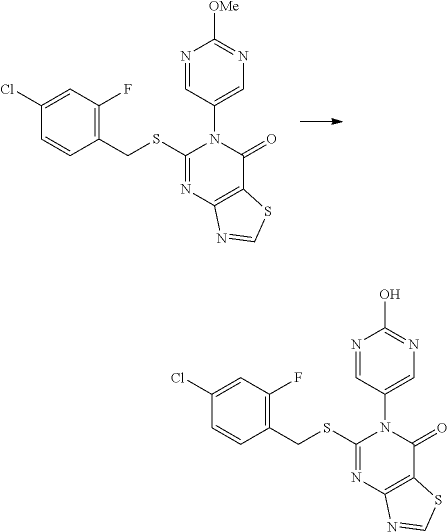 [SMILES: COc1ncc(-n2c(SCc3ccc(Cl)cc3F)nc3ncsc3c2=O)cn1 ; O=c1c2scnc2nc(SCc2ccc(Cl)cc2F)n1-c1cnc(O)nc1]

Trimethylsilyl chloride (92.2 μL, 727 μmol, 4.50 equiv) is added to mixture of 5-((4-chloro-2-fluorobenzyl)thio)-6-(2-methoxypyrimidin-5-yl)thiazolo[4,5-d]pyrimidin-7(6H)-one (intermediate 39, 70.0 mg, 161 μmol, 1 equiv) and potassium iodide (23.0 mg, 145 μmol, 0.90 equiv) in acetonitrile (2 mL). After 18 h, the mixture is concentrated, and the residue is purified by reversed phase HPLC (Waters Sunfire™-C18, gradient of acetonitrile in water, 0.1% TFA), to yield the desired product
C₁₆H₉ClFN₅O₂S₂ (M=421.0 g/mol)
ESI 422 [M+H]⁺
Rₜ (HPLC) 0.67 min (Method E)

The following compounds are prepared using procedures analogous to those described for example 53, using appropriate starting materials. As is appreciated by those skilled in the art, the analogous examples my involve variations in general reaction conditions.

| | | | HPLC retention time |
|---|---|---|---|
| Example | Structure | ESI-MS | [min] (method) |
| 54 | 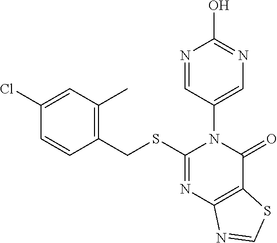 [SMILES: Cc1cc(Cl)ccc1CSc1nc2ncsc2c(=O)n1-c1cnc(O)nc1] | 418 [M + H]⁺ | 0.70 (Method E) |
| 55 | 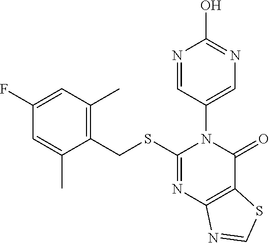 [SMILES: Cc1cc(F)cc(C)c1CSc1nc2ncsc2c(=O)n1-c1cnc(O)nc1] | 416 [M + H]⁺ | 0.66 (Method E) |
| 56 | 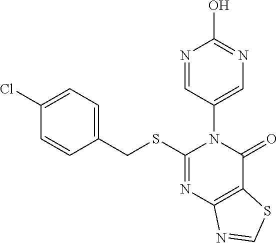 [SMILES: O=c1c2scnc2nc(SCc2ccc(Cl)cc2)n1-c1cnc(O)nc1] | 404 [M + H]⁺ | 0.65 (Method E) |
| 57 | 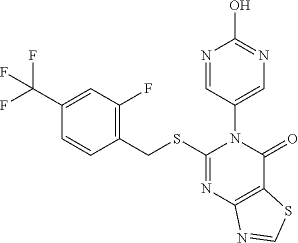 [SMILES: O=c1c2scnc2nc(SCc2ccc(C(F)(F)F)cc2F)n1-c1cnc(O)nc1] | 456 [M + H]⁺ | 0.71 (Method E) |
| 58 | 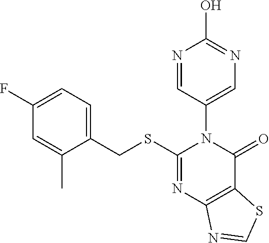 [SMILES: Cc1cc(F)ccc1CSc1nc2ncsc2c(=O)n1-c1cnc(O)nc1] | 402 [M + H]⁺ | 0.76 (Method A) |
| 59 | 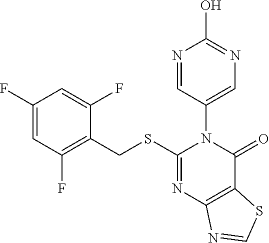 [SMILES: O=c1c2scnc2nc(SCc2c(F)cc(F)cc2F)n1-c1cnc(O)nc1] | 424 [M + H]⁺ | 0.74 (Method A) |
| 60 | 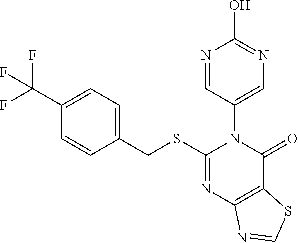 [SMILES: O=c1c2scnc2nc(SCc2ccc(C(F)(F)F)cc2)n1-c1cnc(O)nc1] | 438 [M + H]⁺ | 0.70 (Method E) |

### Example 61

### 5-((4-Chloro-2-fluorobenzyl)thio)-6-(6-hydroxypyridazin-3-yl)thiazolo[4,5-d]pyrimidin-7(6H)-one

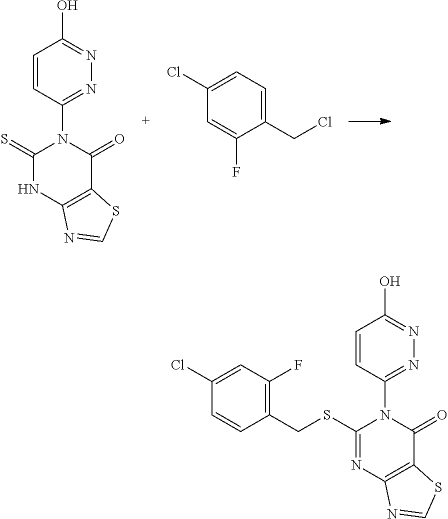 [SMILES: O=c1c2scnc2[nH]c(=S)n1-c1ccc(O)nn1 ; Fc1cc(Cl)ccc1CCl ; O=c1c2scnc2nc(SCc2ccc(Cl)cc2F)n1-c1ccc(O)nn1]

4-Chloro-1-(chloromethyl)-2-fluorobenzene (14.3 mg, 80.0 μmol, 1 equiv) is added to a solution of 6-(6-hydroxypyridazin-3-yl)-5-thioxo-5,6-dihydrothiazolo[4,5-d]pyrimidin-7(4H)-one (intermediate 49, 22.3 mg, 80.0 μmol, 1 equiv) and N,N-diisopropylethylamine (25.0 μL, 145 μmol, 1.80 equiv) in N,N-dimethylformamide (2 mL). After 18 h, the reaction mixture is filtered and the filtrate is purified by reversed phase HPLC (Waters Sunfire™-C18, gradient of acetonitrile in water, 0.1% TFA) to yield the desired product. C₁₆H₉ClFN₅O₂S₂(M=421.0 g/mol)
ESI 422 [M+H]⁺
Rₜ (HPLC) 0.74 min (Method G)

The following compounds are prepared using procedures analogous to those described for example 61, using appropriate starting materials. As is appreciated by those skilled in the art, the analogous examples my involve variations in general reaction conditions.

| | | | | HPLC |
|---|---|---|---|---|
| | | | | retention time |
| | Starting | | | [min] |
| Example | material | Structure | ESI-MS | (method) |
| 62 | 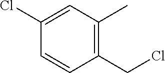 | 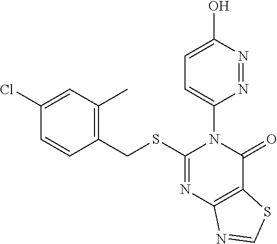 [SMILES: Cc1cc(Cl)ccc1CSc1nc2ncsc2c(=O)n1-c1ccc(O)nn1] | 418 [M + H]⁺ | 0.77 (Method G) |
| 63 | 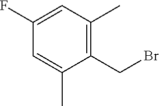 | 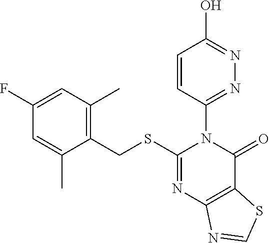 [SMILES: Cc1cc(F)cc(C)c1CSc1nc2ncsc2c(=O)n1-c1ccc(O)nn1] | 416 [M + H]⁺ | 0.76 (Method G) |
| 64 | 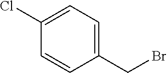 | 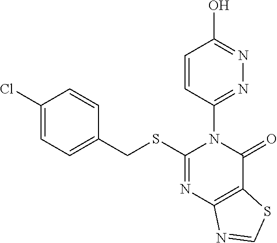 [SMILES: O=c1c2scnc2nc(SCc2ccc(Cl)cc2)n1-c1ccc(O)nn1] | 404 [M + H]⁺ | 0.72 (Method G) |
| 65 | 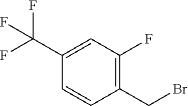 [SMILES: Fc1cc(C(F)(F)F)ccc1CBr] | 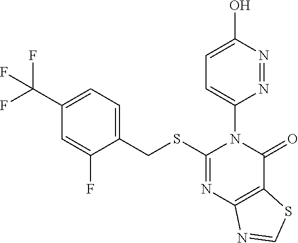 [SMILES: O=c1c2scnc2nc(SCc2ccc(C(F)(F)F)cc2F)n1-c1ccc(O)nn1] | 456 [M + H]⁺ | 0.78 (Method G) |
| 66 | 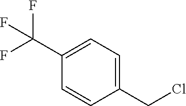 [SMILES: FC(F)(F)c1ccc(CCl)cc1] | 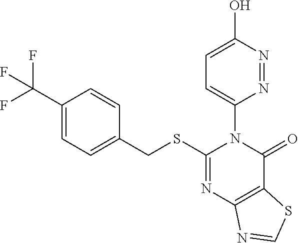 [SMILES: O=c1c2scnc2nc(SCc2ccc(C(F)(F)F)cc2)n1-c1ccc(O)nn1] | 438 [M + H]⁺ | 0.77 (Method G) |
| 67 | 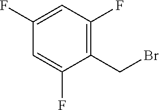 [SMILES: Fc1cc(F)c(CBr)c(F)c1] | 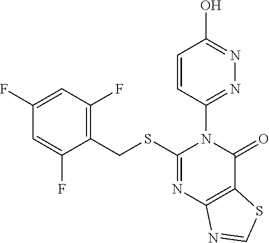 [SMILES: O=c1c2scnc2nc(SCc2c(F)cc(F)cc2F)n1-c1ccc(O)nn1] | 424 [M + H]⁺ | 0.47 (Method C) |
| 68 | 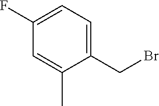 | 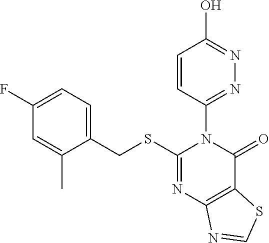 [SMILES: Cc1cc(F)ccc1CSc1nc2ncsc2c(=O)n1-c1ccc(O)nn1] | 402 [M + H]⁺ | 0.71 (Method G) |

### Example 69

### 6-(5-Hydroxypyrazin-2-yl)-5-((2,4,6-trifluorobenzyl)thio)thiazolo[4,5-d]pyrimidin-7(6H)-one

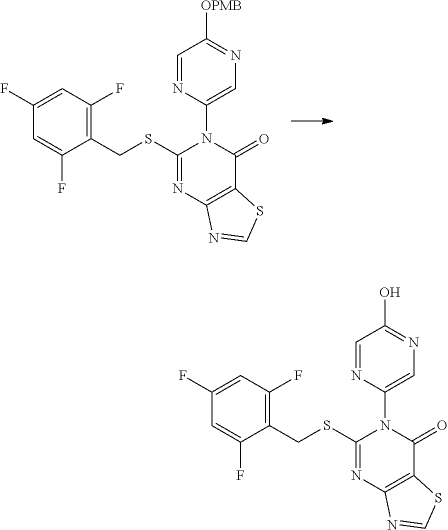

Trifluoroacetic acid (1.00 mL, 13.0 mmol, 235 equiv) is added to a solution of 6-(5-((4-methoxybenzyl)oxy)pyrazin-2-yl)-5-((2,4,6-trifluorobenzyl)thio)thiazolo[4,5-d]-pyrimidin-7(6H)-one (intermediate 52, 30.0 mg, 55.0 μmol, 1 equiv) in dichloromethane (2 mL). After 1 h, the reaction mixture is concentrated, and the residue is purified by reversed phase HPLC (Waters Sunfire™-C18, gradient of acetonitrile in water, 0.1% TFA) to yield the desired product.
C₁₆H₈F₃N₅O₂S₂(M=423.0 g/mol)
ESI 424 [M+H]⁺
Rₜ (HPLC) 0.69 min (Method E)

### Analytical HPLC Methods

### Method A

| time | Vol % water | | Flow |
|---|---|---|---|
| (min) | (incl. 0.1% TFA) | Vol % ACN | [mL/min] |
| 0.0 | 97.0 | 3.0 | 2.2 |
| 0.2 | 97.0 | 3.0 | 2.2 |
| 1.2 | 0.0 | 100.0 | 2.2 |
| 1.25 | 0.0 | 100.0 | 3.0 |
| 1.4 | 0.0 | 100.0 | 3.0 |

Device description: Agilent 1200; Analytical column: Sunfire (Waters) C18_3.0×30 mm_2.5 μm; column temperature: 60° C.

### Method B

| time | Vol % water | | Flow |
|---|---|---|---|
| (min) | (incl. 0.1% FA) | Vol % ACN | [mL/min] |
| 0.0 | 97.0 | 3.0 | 2.2 |
| 0.2 | 97.0 | 3.0 | 2.2 |
| 1.2 | 0.0 | 100.0 | 2.2 |
| 1.25 | 0.0 | 100.0 | 3.0 |
| 1.4 | 0.0 | 100.0 | 3.0 |

Device description: Agilent 1200; Analytical column: Sunfire (Waters) C18_3.0×30 mm_2.5 μm; column temperature: 60° C.

### Method C

| time | Vol % water | | Flow |
|---|---|---|---|
| (min) | (incl. 0.1% TFA) | Vol % ACN | [mL/min] |
| 0.00 | 99 | 1 | 1.6 |
| 0.02 | 99 | 1 | 1.6 |
| 1.00 | 0 | 100 | 1.6 |
| 1.10 | 0 | 100 | 1.6 |

Device description: Waters Acquity; Analytical column: Xbridge (Waters) BEH C18_2.1×30 mm_1.7 μm; column temperature: 60° C.

### Method D

| time | Vol. % water | | Flow |
|---|---|---|---|
| (min) | (incl. 0.1% NH3) | Vol. % ACN | [mL/min] |
| 0.00 | 95 | 5 | 1.5 |
| 1.30 | 0 | 100 | 1.5 |
| 1.50 | 0 | 100 | 1.5 |
| 1.60 | 95 | 5 | 1.5 |

Device description: Waters Acquity; Analytical column: XBridge (Waters) C18_3.0×30 mm_2.5 μm; column temperature: 60° C.

### Method E

| time | Vol. % water | Vol % ACN | Flow |
|---|---|---|---|
| (min) | (incl. 0.1% TFA) | (incl. 0.1% FA) | [mL/min] |
| 0.00 | 95 | 5 | 1.5 |
| 1.30 | 0 | 100 | 1.5 |
| 1.50 | 0 | 100 | 1.5 |
| 1.60 | 95 | 5 | 1.5 |

Device description: Waters Acquity; Analytical column: Sunfire (Waters) C18_3.0×30 mm_2.5 μm; column temperature: 60° C.

### Method F

| time | Vol % water | | Flow |
|---|---|---|---|
| (min) | (incl. 0.1% NH₃) | Vol % ACN | [mL/min] |
| 0.00 | 97 | 3 | 2.2 |
| 0.20 | 97 | 3 | 2.2 |
| 1.20 | 0 | 100 | 2.2 |
| 1.25 | 0 | 100 | 3.0 |
| 1.40 | 0 | 100 | 3.0 |

Device description: Agilent 1200; Analytical column: Xbridge (Waters) C18_3.0×3 mm_2.5 μm; column temperature: 60° C.

### Method G

| time | Vol % water | | Flow |
|---|---|---|---|
| (min) | (incl. 0.1% TFA) | Vol % ACN | [mL/min] |
| 0.0 | 95.0 | 5.0 | 1.5 |
| 1.3 | 0.0 | 100.0 | 1.5 |
| 1.5 | 0.0 | 100.0 | 1.5 |
| 1.6 | 95.0 | 5.0 | 1.5 |

Device description: Waters Acquity; Analytical column: Sunfire C18 (Waters) 3.0×30 mm_2.5 μm; column temperature: 60° C.

1. A compound of formula (I) 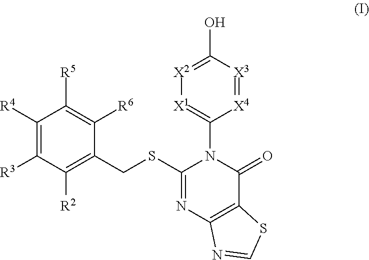
wherein
X¹, X², X⁴ are, independently of each other, N or C—R¹;
X³ is N or C—R¹⁻¹;
provided that not more than two of X¹, X², X³, X⁴ are N simultaneously;
R¹ is —H or -halogen;
R^{1.1} is selected from among a group consisting of —H, -halogen and —CN;
R², R³, R⁴, R⁵, R⁶ are, independently of each other, selected from among a group consisting of —H, -halogen, —CF₃ and -methyl;
or a salt thereof.

2. A compound according to claim 1, wherein at least one of X¹, X², X³, X⁴ is —N; or a salt thereof.

3. A compound according to claim 1, wherein
X¹, X², X⁴ are C—R¹;
X³ is C—R^{1.1};
R¹ is —H, —F or —Cl;
R¹⁻¹ is —H, —F or —Cl;
or a salt thereof.

4. A compound according to claim 1, wherein
X² is N;
X¹ and X⁴ are —C—R¹;
X³ is C—R^{1-1;}
R¹ is —H, —F or —Cl;
R¹ is —H, —F or —Cl;
or a salt thereof.

5. A compound according to claim 1, wherein
X² and X³ are N;
X¹ and X⁴ are —C—R¹;
R¹ is —H, —F or —Cl;
or a salt thereof.

6. A compound according to claim 1, wherein
X¹ and X² are N;
X³ is C—R^{1-1;}
X⁴ is C—R¹;
R¹ is —H, —F or —Cl;
R^{1.1} is —H, —F or —Cl;
or a salt thereof.

7. A compound according to claim 1, wherein
R¹ is —H or —F;
R^{1.1} is —H or —F;
R³ and R⁵ are —H;
R², R⁴, R⁶ are, independently of each other, selected from among a group consisting of —H, -halogen, —CF₃ and -methyl;
or a salt thereof.

8. A compound according to claim 1, selected from the group consisting of: 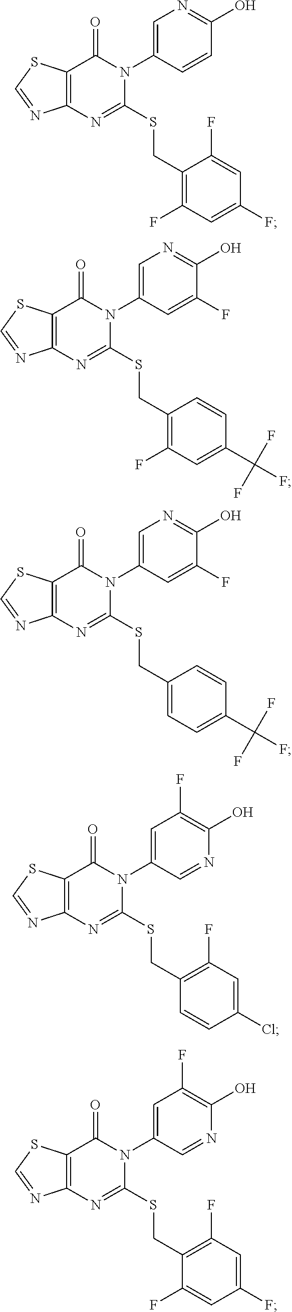 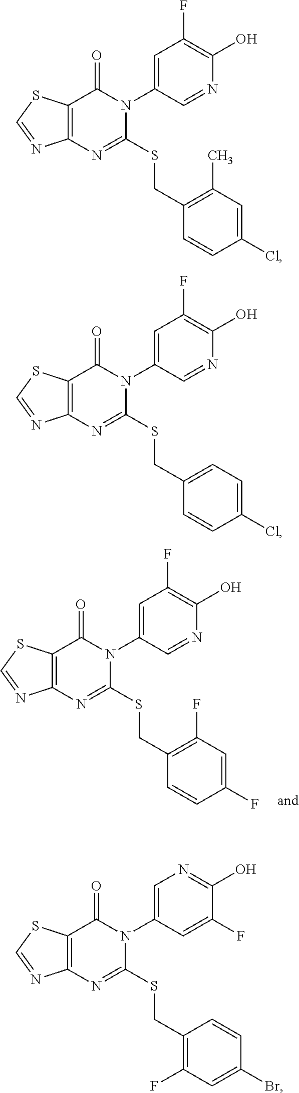
or a pharmaceutically acceptable salt thereof.

9. A compound according to claim 1 in its salt free form.

10. A method for the treatment and/or prevention of a disease selected from among the group consisting of chronic liver diseases, portal hypertension, viral infections, cancer, interstitial lung diseases, retinopathies, acute and chronic inflammation, and fibrotic diseases, which method comprises the administration of an effective amount of a compound of formula (I) according to claim 1, or a pharmaceutically acceptable salt thereof, to a human being.

11. A method according to claim 10, wherein the disease is selected from among the group consisting of vascular inflammation, atherosclerosis, interstitial lung diseases, liver fibrosis, pulmonary hypertension, portal hypertension, liver cirrhosis, acute on chronic liver failure (ACLF), sepsis, multi-organ failure, diabetic retinopathies, wet age-related macular degeneration (AMD), dry AMD, cardiovascular diseases, NOX4+ cancer associated fibroblast rich tumors, systemic sclerosis, inflammatory bowel disease, Duchenne muscular dystrophy, COVID-19, acute respiratory distress syndrome and influenza.

12. A pharmaceutical composition comprising at least one compound according to claim 1, or a pharmaceutically acceptable salt thereof, and a pharmaceutically acceptable carrier.

13. A pharmaceutical composition comprising one or more compounds according to claim 1, or a pharmaceutically acceptable salt thereof, and a further active substance selected from the group consisting of antifibrotics, immunotherapeutics, sGC activators, ATX-inhibitors, SGLT2-inhibitors, THRb inhibitors, GLP1 agonists and GLP1 agonist combinations, FGF-analogs, KRAS-G12C-inhibitors, KRAS-G12D-inhibitors, MDM2-p53-antagonists, of Her2-inhibitors and chemotherapeutics.
